SEQUENCE LISTING (108 sequences)

SEQ ID NO: 1 - DNA, 1599 bp, Rauvolfia serpentina
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 2 - DNA, 1605 bp, Gelsemium sempervirens
atggcaacaccaagctcaactattgtccccgacgccacgaagatcaatcgtagagattttccgagtgattttgtgtttggtgcggctagctcagcataccagatagaaggtggtgccagtgagggtggcaggggaccctccatctgggatacatttactaaaagaagacctgagatggtaaaaggaggatccaatggaaacgtggctattgatagttaccacttatacaaggaggatgttaagattctaaagaacctgggtttagacgcatatagattttctatatcctggtcaagaatccttcccggcggtaatcttagcggaggtattaataaggaggggatagacttctacaacaattttatcgacgagttgatcgcctcaggaatccaaccctacgttacattattccattgggatgtgccgcaagccttagaagatgaatacggcggcttcctaagtccgaagatagttgacgattttagggattatgctgagttgtgcttctggaatttcggcgacagggtcaagaattggatcaccctaaacgagccgtggactttctctgtcgacggctatgtcgctggaacgttcgcccccggaaggggcgcaacaccaactgaccaagtaaaaggacccattaaaaggcacaggtgttcaggatgggggccacaatgctcaaatagtgacggaaaccccggcacagaaccgtatttagtgacccaccaccagattctagcgcatgctgcagccgtcgaatcatataggaacaaattcaaggcgagccaggaaggtcagatagggatcacgatagtcgctcagtggatggaaccattgaacgagaaatctgattcagatgtccaagcagcgaagagggcccttgacttcatgtatggatggttcatggaaccaatcacatcaggggattacccagaaataatgaagaagatcgtaggttctaggttacccaaattttcagcggaacagtcaagaaagctgaagggtagttatgactttctgggcttaaactactacacagcgaactacgttaccagcgcacctaaccccaccggtggtatagtatcttatgatacagatacccaggtgacttaccactcagataggaatggaaagttaataggaccactagccggctcagagtggctgcacatttacccggagggtataagaaagttactagtgtatacgaagaaaacgtacaatgttccgttgatctacataacagaaaatggcgtagacgagttgaacgatactagcttgacattgagtgaggccagggtagacccgataagaattaagttcatacaagaccatctactgcagctacgtttagcaattgatgacggggtaaacgtaaaaggctattttgtctggagtttgttagacaatttcgaatggaacgaaggattcacggtaaggttcggcatgattcacgtaaattataacgaccaatacgcacgttatccgaaagatagcgcgatttggctgatgaacaacttccataaaaagtttagcgggccgcccgttaaacgtagtgtcgaagagaatcaggaaactgacagtcgtaaaagatcccgtaagtaa

SEQ ID NO: 3 - DNA, 1431 bp, Scedosporium apiospermum
atgtcccttccaaaagacttcttatgggggttcgcgactgcggcataccagattgaaggcgcttccgaaaaggatgggagagggccgagcatatgggacaccttttgtgcgataccagggaagatagctgatggcagtagcggcgccgtggcatgcgactcctacaatagagctggtgaagatatcgcactattaaaagaactaggcgcaagcgcatatagattttccataagttggtcaagaataattccgctagggggtagaaacgatcccgtgaatcaggccgggattgaccattacgtcaaatttgtcgacgatcttacagacgctggcataactccctttgtaaccctatttcactgggatcttcctgacggtctggataagagatatgggggcctactgaacagggaggaatttccacttgacttcgagcattacgccagaacggttttcaaagcactacctaaggtgaagcactggattacctttaacgagccgtggtgcagtgctatcttagggtataatacaggtttctttgctcctggtcacacgtccgacagaacgaaatctgccgtcggagacagcgctagagagccatggattgccggccacaatatgctagtggctcatggaagagctgtaaaggcttacagggaagaattcaagcctaccaatggaggggagataggtattacactaaatggggacgccacatatccatgggatcccgaagaccccgaagacgttgccgcatgcgatagaaagatagaattcgctatttcctggtttgctgacccaatatatttcggtaagtacccggattctatgttggctcagctgggagatcgtctgccgacattcacagatgaagaaagggctctagtacaagggagtaacgacttctatggaatgaaccactacacagcgaactacattaaacataagacagacacaccacctgaagatgactttcttggtaatctagaaacgttatttgagtcaaagaatggggactgcattggccccgagacacagtcattttggcttaggcctaaccctcaaggattcagagatttactgaattggctgagcaaaagatacgggagacctaaaatttatgttaccgagaacggaacttcaatcaaaggcgagaacgacctgccacgtgaacaaatcctacaagacgatttcagggttgagtacttcgactcatatgctaaagcaatggccgatgcgtacgaaaaagacggcgttgatgtaagaggatacatggcatggagtttattagataattttgaatgggcagaagggtatgagacccgtttcggcgtcacttttgtggattatgcgaacggacaaaaaaggtatccgaagaagtccgcacgttctctaaaaccgttatttgacagcttgattaaaaaggattaa

SEQ ID NO: 4 - DNA, 1611 bp, Rauvolfia verticillata
atggaatccaaccaaggagagcctctggttgtagcaatcgtaccaaagcctaacgcgtctactgagcaaaaaaactcccatttgattccggcgacaaggtctaaaatcgtcgtccacaggcgtgacttccctcaagattttgtatttggtgcgggagggtctgcgtaccaatgtgaaggggcatacaatgaaggtaatcgtggcccatcaatctgggacacatttacacagaggacaccagctaaaatctcagacggatcaaatggaaaccaagctattaactgttaccacatgtataaggaagacataaagataatgaaacaggccggactggaggcgtaccgtttcagcatctcatggtctagggttctaccgggcggtagattagcagccggagttaataaggatggggtgaagttttatcacgacttcatcgacgaattgctggctaatgggattaagccgttcgccactttgttccactgggatttaccgcaagccttagaagacgagtacggtggtttcttaagccatcgtattgttgacgatttttgtgagtatgcagagttttgtttctgggaatttggcgacaaaattaaatactggactacttttaatgagccacatacattcacagctaacggctacgctctgggggaatttgctcccggtagaggtaaaaatggcaagggcgacccagccacagaaccgtatctggttactcacaatattttactggcccataaagccgccgtagaggcttaccgtaataagttccaaaaatgccaggaaggcgaaataggcatagtcttgaatagcacgtggatggagcctctgaatgatgtgcaggctgatattgatgctcacaagagagcgttagacttcatgctagggtggtttatagaacccttgaccaccggcgactatcccaagagtatgagggagattgttaagggtcgtttacctcgtttctcaccagaggatagcgagaagctgaaggggtgctatgatttcgtcggcatgaattactataccgctacctacgtcaccaatgcggcgaagagtaattctgagaagctaagctacgagacagacgaccacgtcgacaaaactttcgatagggtcgttgatgggaaatctgtcccaatcggtgccgtgttgtatggtgagtggcaacacgttgtaccctggggcttatacaaactattggtttacacaaaggaaacataccacgtccccgtactgtacgtgaccgagagcgggatggtcgaagaaaacaagactaagatccttctgagtgaggccagacgtgaccccgaaagaacggactatcaccagaagcatttggcgagcgtacgtgatgcgatagatgacggtgtgaacgtgaaaggctacttcgtatggagcttcttcgataattttgagtggaatctgggatttattggcagatacgggattattcatgtggattacaatagtttcgagagatgtccgaaagagtcagccatttggtataagaattttatagcgggcgtttccacgacgagcccggccaagcgtcgtagggaagaggcggagggagtcgagcttgtcaaaaggcagaagacataa

SEQ ID NO: 5 - DNA, 1071 bp, Chatharanthus roseus
atggctatggctagtaagagcccttctgaggaggtctatccagtaaaagcattcgggctggcagcgaaagactcctccggactattttcaccattcaacttctctaggagggccacgggtgagcacgatgtacagctaaaggttttatattgcggtacctgtcaatacgatcgtgagatgtcaaaaaataagttcggcttcacaagctatccgtacgtacttggacacgagatagtgggtgaggttacagaggtggggtccaaagtacagaagttcaaagtgggggataaagtcggggtcgcctcaattattgaaacgtgcggcaagtgtgaaatgtgcacaaatgaagtggagaattactgtcctgaggcaggatcaatcgacagcaactatggtgcatgctccaacatcgctgtcatcaatgagaattttgtcattcgttggcctgagaacctgcctttagattcaggcgtgcccttgttgtgcgcgggtattacggcttattctcccatgaagagatatggactagacaaaccgggaaagagaattggtatagccggcttgggaggtttgggtcatgtcgcgctacgttttgcaaaggcgtttggcgcgaaagtaaccgtcattagttcatcacttaaaaagaagagggaggcgtttgaaaagttcggggcagattcattcttggtgtccagtaaccctgaagaaatgcaaggggccgcgggaactcttgacgggataattgacactatccctggaaaccatagcctagagcccttactagcgttgttgaaacccttaggaaagctaattattctgggtgccccggagatgccatttgaggttccagcgccgtcattattgatgggaggcaaggtgatggctgcgtcaacggccggtagtatgaaagaaatccaggaaatgattgagtttgcagcagaacacaatatcgttgctgacgtcgaagttattagtattgattatgtgaacaccgcgatggaacgtcttgacaactctgatgtgcgttacaggtttgtcatagacatcgggaacactctgaagagtaattaa

SEQ ID NO: 6 - DNA, 1494 bp, Gelsemium sempervirens
atgcagctgtctttttcttatcccgcattgttcctattcgtttttttcttgtttatgttggtcaagcaattgaggcgtcctaagaatctgccgccggggccaaataagttgccaatcattggcaacttgcaccaactagccacagaattgccacaccatacacttaaacaactggcagacaagtatggtcccattatgcatttacagtttggcgaggtatcagccatcatagtaagctctgctaagctagcaaaggttttcctaggaaaccatggacttgctgtcgctgataggcctaaaacgatggtcgcgacaataatgttgtacaatagtagcggtgtcaccttcgcgccgtatggtgattactggaaacatttaagacaggtgtatgcagtggaattattgagccctaagagcgttcgtagtttctccatgataatggatgaagagatatccctaatgttaaagagaatacagtctaatgccgctggacagccgcttaaggttcacgatgaaatgatgacatacttattcgcgacactgtgcagaactagcatcggatctgtttgtaagggtcgtgacctgctaatagataccgcaaaggacattagtgcaatttccgccgcgatcaggatcgaagaattgttcccttctctaaaaatacttccctacattactggcttacaccgtcaattggggaagctttcaaagaggctggacggtatcttagaagacatcatcgctcagagggaaaaaatgcaggagtctagcacaggagataacgatgagcgtgacatactgggggtgcttctgaagttgaagcgttccaattccaatgataccaaagtgagaatccgtaatgatgacataaaagcaattgtgttcgagttgattcttgctgggacgttaagtaccgctgctacggtagaatggtgcctgagcgagctaatgaaaaatccgggagccatgaaaaaagcccaggatgaggtgaggcaagtgatgaagggcgagactatctgcaccaatgacgttcagaagttagaatatataaggatggttatcaaggaaacattcaggatgcacccgccagccccacttcttttcccacgtgagtgtcgtgaacctatccaagtcgagggatatacaattcctgaaaagagctggctaatagtcaactactgggctgtaggtcgtgatccagaactttggaatgaccctgagaagtttgagccagaaagattcaggaatagtccggtcgatatgagtggtaaccactacgagcttatacccttcggtgctggcaggaggatttgccctgggatttctttcgcggcaactaacgcggagctgctgttagcatctttaatataccatttcgattggaaattaccggctggggttaaggagcttgacatggacgaactgttcggtgcaggttgcgtgcgtaaaaaccccttacacttgataccgaagacggttgtgccactgagttaa

SEQ ID NO: 7 - DNA, 1059 bp, Catharanthus roseus
atggcaaatttctcagaatccaaatcaatgatggctgtcttttttatgttctttctgttgctgttatcatcctcatcttcatcatcctcctcaagtcctattttgaaaaagatattcattgaatctccaagctatgctccaaacgcctttacttttgatagtactgacaaaggcttttacacttcagtgcaagatggtagagttattaaatatgagggtcctaattctggctttacagattttgcttacgcatccccattttggaacaaagctttttgcgaaaatagtacagatccggaaaaaagaccactatgtggtagaacatatgatatctcatatgattacaagaacagtcaaatgtacattgttgatggtcactaccatttgtgtgtcgtcggtaaagaaggtggatatgctacgcaattagctacgtcagtgcaaggagtccctttcaaatggctatatgcggtgaccgtcgatcaaaggactggtatcgtatatttcactgatgtcagctctatacacgacgatagcccagaaggggttgaagaaattatgaatacttcagataggactgggagactgatgaagtacgacccatctaccaaggaaaccacattattgttaaaggaactacacgtaccaggaggtgccgaaatctctgctgatggctccttcgtcgttgtagctgaattcctatcaaacagaatcgtaaaatattggttagaaggtccaaagaaaggttctgctgaattcttagtaacgattcccaaccctggaaacattaagagaaatagtgatgggcatttctgggtaagttcttccgaagaacttgacggaggtcaacatggtagagttgtttccagaggtataaagttcgatggatttggcaacatattgcaagtcatccctcttccaccgccttacgaaggcgaacattttgaacaaatacaagaacatgatggtttattgtacattggaagcctgttccattcaagtgttggaattttagtttacgatgatcacgacaataaaggtaactcatacgtcagttcataa

SEQ ID NO: 8 - DNA, 2145 bp, Catharanthus roseus
atggactcatcctccgagaagttgtcaccattcgaacttatgtcagcaattcttaagggagccaagctggacggtagtaacagttctgattccggtgtcgctgtatcacctgctgttatggcaatgttactagaaaataaagagttagtaatgatattgacgacatctgtcgctgtcttgattggttgcgtcgttgtgctaatttggcgtagatcttcagggtccggtaagaaggttgtggagccacccaagttgatagtcccaaaaagtgtagtggagccagaagaaatagatgaaggaaaaaaaaaattcactatcttctttggtacacaaactgggacagctgaaggttttgctaaggctttagccgaagaagcaaaggctagatacgaaaaggcagttataaaagtaatcgatattgacgattatgcagcagacgatgaggagtatgaggaaaaattcagaaaagagactttggccttctttatattggcaacatatggcgatggtgagcctactgataacgctgcaaggttttacaaatggtttgtagagggtaatgatagaggtgactggcttaagaacttacagtatggcgtcttcggtttgggcaatagacagtatgaacatttcaataagattgcaaaagttgtagatgaaaaggttgccgagcaaggagggaagaggatagtgcctttagttttaggagacgatgatcaatgtattgaagatgactttgctgcatggagagaaaacgtctggcctgaactggataatctgctaagagacgaggatgatactacagtgtctactacctatacagccgctataccagaatacagagttgttttccctgataaaagtgattctttgatttctgaggccaacggccacgctaacggctatgcgaacggcaatactgtatacgatgctcaacacccttgccgtagtaacgtcgctgtcaggaaagagttacataccccagcttctgataggtcttgtacacatttggattttgatatagcgggtactggattatcatatggtacaggagatcacgtcggtgtctattgtgacaatttatctgagaccgtagaagaagcagaaaggttgttgaacttgccccctgaaacgtatttcagtttgcacgctgataaagaagatggtactccattagcaggatcatcattgccccctccttttcctccgtgcacattgaggactgcccttactagatatgctgatctgctgaataccccaaaaaagtccgccttgctggctctagctgcttatgcaagcgatccaaatgaggctgatcgtttgaagtacttggcaagcccagctggcaaagacgagtatgctcaatctttggtagctaatcagagaagcctgctagaagtaatggctgaatttccttccgccaagccaccgttaggtgtattcttcgcagcaatagctcccagattacaacccagattctactctatatcttctagcccaaggatggccccttccagaattcatgtcacttgcgctctagtttatgagaaaactccaggcgggaggattcataaaggcgtatgttcaacttggatgaagaacgctattcctctggaggaatctcgtgattgtagctgggcaccgatctttgtgagacagtctaactttaagctgcctgccgatccaaaagttcctgtaatcatgataggtccaggcaccgggctagcaccttttagaggtttccttcaagaaagacttgctctgaaagaggaaggagctgaattaggaactgctgtatttttttttgggtgtaggaacagaaaaatggattatatatatgaagatgaattgaatcatttcttggaaatcggcgcgttatcagaattgctggttgcattcagtagggaaggtcctactaagcaatatgttcaacacaaaatggccgaaaaagccagtgatatttggcgtatgatctctgatggtgcttatgtttatgtctgcggagatgccaagggcatggccagagacgttcataggacattacacactatagctcaagagcaaggatcaatggactctactcaggccgaaggatttgtgaaaaacttacaaatgaccggtagatatttaagagacgtatggtaa

SEQ ID NO: 9 - DNA, 405 bp, Catharanthus roseus
atggcctctgatcaaaagttgcataagttcgatgaagtctcaaaacataataaaacgaaagattgttggctgattattaatggtaaggtctacgacgtcactccgtttatggacgatcatccaggtggtgacgaagtcttattatccgccacaggcaaggacgcaacaaatgactttgaagatgttggtcactctgacagcgctagagaaatgatggataaatattacattggtgagatggatatggctactgttccacttaaaagaacatacattcctccacagcaagctcaatataatcctgacaagacaccagagttcgtgattaagatccttcaatttttagtacccttgctgatattgggtttagcgttcgctgttagacattacaccaaggaaaaataa

SEQ ID NO: 10 - DNA, 1095 bp, Catharanthus roseus
atggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaataa

SEQ ID NO: 11 - DNA, 1506 bp, Catharanthus roseus
atggagttttccttttcttcccccgctttgtatatagtgtattttctgttgttcttcgttgttaggcagttgctgaaacccaaatcaaagaagaaactaccaccaggcccaagaacgctgcctctgatagggaatttacatcagttgagcggaccattgccgcaccgtacattaaagaacctatcagataaacacggtccgctgatgcacgtgaagatgggcgagagatctgccatcatagttagcgacgcaaggatggcgaagatagtcttgcacaataacggattggccgttgcagataggtcagtcaatactgtcgcgtccattatgacctacaactcactgggcgtcacgtttgctcaatatggcgactacctgaccaaattgcgtcagatctataccttggagctactttcccagaagaaagtcagaagtttttattcttgtttcgaggacgaactagacactttcgtaaagtctatcaagtccaatgtgggccagccgatggttttgtacgaaaaagcatctgcgtatttgtatgccacaatttgtagaaccatcttcgggagcgtttgcaaagaaaaagagaagatgataaaaatagtcaagaaaaccagcctattgagcgggactcctctaagactagaagacttgtttccaagcatgtctattttctgtcgtttttctaagactctgaatcagctgagaggcctgcttcaagaaatggacgatatccttgaagagatcatagttgagcgtgaaaaagcatctgaggtttcaaaagaagcgaaagacgatgaagacatgttaagtgtactactgcgtcacaaatggtataatccaagtggagccaaatttagaatcaccaatgctgatatcaaagctataatctttgaacttatacttgcggcaacgctatcagtggcagatgttacggaatgggcaatggttgaaatcttacgtgatccgaagtctcttaagaaagtatatgaggaggtacgtggcatttgtaaagagaaaaagagggtcacaggatatgacgtggagaagatggagttcatgcgtttgtgcgttaaagaatccactagaattcatccagctgcaccattgttagttccccgtgaatgtcgtgaggattttgaggttgatgggtacacagtccccaagggcgcatgggtgataaccaactgttgggcggttcagatggaccccacagtctggcccgagcctgaaaaattcgatcctgaacgttatattcgtaaccccatggacttctatggatctaattttgagctaatcccatttggtaccggcaggagaggctgccccggcatattgtatggcgttactaacgcagaatttatgttagctgctatgttttatcactttgattgggagatagccgatggtaagaaaccggaagaaattgacctgacggaagatttcggtgctggctgcataatgaagtacccactaaagttagttccgcatttagttaatgactaa

SEQ ID NO: 12 - DNA, 1065 bp, Catharanthus roseus
atggccgacagggtgaagactgttggatgggctgcacacgactcctctggattcttatctccatttcaattcacgagaagggctaccggggaggaagacgttaggttgaaagtgctatattgcggggtatgccattcagacctacataacatcaaaaatgaaatgggttttacgtcctacccctgcgtccctggacacgaggtagtgggagaggtaacggaagttggaaataaagtaaagaaattcataattggtgacaaagtcggggtagggttgtttgtggatagctgtggagagtgtgaacaatgcgttaacgatgttgagacttactgcccgaaacttaaaatggcatatttaagtatcgacgacgatggcacggttattcagggtgggtatagcaaagaaatggttataaaggagaggtatgtttttcgttggccggagaaccttcccttgccagcgggaacccccttactaggggctggttctactgtgtacagcccaatgaaatactacgggctagataagagtggccaacatttgggagtcgttggcctgggggggctgggccacctggctgtaaagtttgctaaggcatttggtcttaaagtcactgtaatttccacatccccatctaaaaaggacgaggccatcaaccatcttggggctgacgccttccttgttagcactgaccaggaacagactcaaaaagctatgagcaccatggacggaatcatagacactgttagtgccccacatgctcttatgccccttttctcactgttgaagcctaacggaaagttgatcgtcgtaggcgctcccaataaacctgtagagttagatatattgtttctagtaatgggtagaaaaatgttaggaacctctgcagtaggtggagtcaaggagacacaggaaatgattgacttcgcagcgaagcacggaattgttgctgatgtggaagtggtggagatggaaaatgttaataacgcgatggaaagactagccaaaggtgatgttaggtatcgttttgtattagatataggtaatgcgacagtcgcagtttaa

SEQ ID NO: 13 - DNA, 972 bp, Catharanthus roseus
atggaaaagcaagttgagatacctgaggtcgagttaaactccggccacaagatgcctatcgttggatatgggacctgtgtcccggaaccaatgccaccgttagaggaacttaccgctattttcctggacgctattaaggttgggtaccgtcacttcgacactgcgtcttcttatggaaccgaagaagctcttggaaaggcaatagccgaagcgattaactcagggttggtcaaatcccgtgaagaattctttatttcctgtaagttatggatcgaagatgccgaccatgacttaatacttcctgccttaaaccagagtcttcaaattcttggggtggactacttagacctatacatgatccatatgccagtgagggtccgtaaaggcgcacctatgttcaactatagtaaagaagacttcctgccatttgacattcaggggacatggaaagcgatggaggagtgcagcaaacaaggtttagccaaaagcatcggtgtatccaactactccgtggaaaaacttacgaaattactagagacatccaccatcccccctgccgttaaccaagtcgaaatgaatgtcgcttggcaacaaaggaaactattaccgttctgtaaggagaaaaacatacacatcaccagttggagccctttactatcctacggcgtcgcttggggtagcaacgccgtcatggagaatcctgtgttacagcaaattgccgctagtaaagggaagacagtggcacaggttgcactgcgttggatatacgagcagggcgctagcctgatcacaaggacgagtaataaggatagaatgtttgagaacgtgcagatatttgactgggaattgtccaaagaagagctagaccaaatacacgaaattccccaacgtcgtggaacgcttggggaggaattcatgcacccggaaggcccaattaaaagtccggaggagttatgggatggtgatttataa

SEQ ID NO: 14 - DNA, 1266 bp, Catharanthus roseus
atggctcctcagatgcagattctgtccgaggaattgatccagcctagctccccgacaccccaaacgttaaagacacataaactaagtcatctggaccaggtgctactgacttgccatatccccattattttattttacccgaatcaattagactcaaacttagacagggcgcagagatcagaaaacttgaaacgttcactatctactgtactgacgcagttctacccactggcgggaaggataaacataaatagttccgtggattgtaatgattcaggagttccttttctggaggcccgtgtccactcacagctaagtgaggcaataaagaacgtggcaatcgacgaattaaaccagtatctaccattccagccttatcctggaggagaggaatctggactaaaaaaggacatcccactggccgtaaagataagttgtttcgagtgtggggggacagctataggagtctgcatatctcacaaaatagcggatgcattaagtttggccactttcctaaacagttggacggctacatgtcaagaggagacagatattgtgcaaccgaacttcgacttgggctctcaccatttccccccaatggaaagcattccagcgcctgagtttcttcccgatgaaaatatcgtcatgaaaaggtttgtctttgacaaagagaaacttgaggccttgaaagcacagctagcgtctagtgccactgaagtgaaaaactcatccagggtccagatcgtaattgctgttatatggaaacagttcatagacgttacaagagctaaatttgacacgaaaaacaagcttgtggctgcacaagcagtcaacctgcgtagcagaatgaacccaccatttccgcagtccgcgatgggcaatatagcaaccatggcttacgcagtcgctgaagaggataaggattttagtgatttagtaggcccattgaaaacttcattggcaaaaatcgatgacgaacatgtgaaggagcttcagaagggtgtaacctaccttgattacgaagctgaaccgcaagagcttttctctttttcatcctggtgtaggttaggcttttatgatctggattttggctggggaaagcctgttagtgtttgtacgacaacggtcccgatgaagaatcttgtatacttaatggatacaaggaacgaagacgggatggaagcgtggatcagtatggcggaggatgagatgtcaatgcttagctcagatttcttgtcactactagatactgatttttctaattaa

SEQ ID NO: 15 - DNA, 1590 bp, Catharanthus roseus
atgataaaaaaggtccctatcgttttatccatcttctgttttttgttattactatcttcttcccacggatccattccggaggcgttcctaaattgtatttctaataaattctcattagacgtaagcatattgaacatactgcacgtcccctcaaatagtagttacgactctgtacttaaatccacgatacagaatccgaggttccttaaaagtccgaaaccactagccattattacccctgttctgcacagccatgtacaatccgctgtaatctgtaccaagcaagcgggactacagattagaattagatcagggggagctgactatgaaggcctgagctataggtccgaagtacccttcatactgcttgatttacagaatttacgtagtatttccgtcgacattgaggacaattctgcgtgggtggaaagtggtgcgactataggcgagttctaccacgaaatcgcacaaaacagcccagtgcacgcgttccctgctggagtcagctcatccgttggcatcggtggacacctgtcttccggcgggttcgggactctacttagaaagtacggcttggcagcggacaacattatagatgcgaaaatagtagatgcaaggggtcgtatcttagacagggagtccatgggtgaagacctattctgggctataagagggggaggcggcgcgagttttggggtcattgtgagctggaaagtcaagttagtaaaagtaccaccgatggtgactgtatttattttgagtaaaacatacgaggaaggggggctagatttactgcacaaatggcaatacatcgagcataagctacccgaggatctgttcttagcggtctcaattatggacgacagtagtagcggcaataaaacgctgatggctggctttatgtccctattccttggcaagactgaagacctactgaaggtcatggcggagaactttccccaattaggtctgaagaaagaggattgtctagagatgaattggattgacgcagcgatgtactttagtggccacccaattggtgagagccgttctgtgttgaaaaatagggaaagtcacctaccaaagacttgcgtgagcataaagtccgacttcattcaagaaccacaaagcatggacgccttggagaaattatggaaattctgtagggaggaagagaactctcctatcatattgatgttacccctaggaggtatgatgagtaagatcagcgagtcagagataccttttccctaccgtaaggatgttatttactcaatgatttatgagatagtatggaattgcgaggacgacgaatctagtgaagaatatatcgacggtctgggcaggttggaagagttgatgactccttatgtcaagcaaccgaggggctcctggttctctacaaggaacctttataccggaaaaaacaagggaccgggtactacctacagcaaagcgaaggagtggggatttagatatttcaacaacaacttcaagaaattggcattgatcaaagggcaagtagacccagagaactttttctattatgaacagtccattccacctctgcatcttcaagttgagctataa

SEQ ID NO: 16 - DNA, 1098 bp, Catharanthus roseus
atggcaggcaagagcgcggaggaggaacatcccatcaaggcttatggttgggcagtcaaagacaggacgacaggtatcctgtcccccttcaagttctccaggagagcgaccggggacgacgatgttaggataaaaatactatactgtgggatatgtcacacagatctagcatctatcaagaacgaatatgaattcctatcctatccgctagtacccggaatggaaatagttggaatagcaacagaggttggcaaagatgttactaaagtaaaggtcggtgaaaaggttgctttgagcgcctatttagggtgctgtgggaagtgttatagctgtgtgaacgaactagaaaattactgccctgaggtcattatagggtatggaacaccgtaccatgacggcacgatatgttacggtggattatccaacgagacagttgccaaccagtccttcgttctaagattcccagagagactatctccagccggcggcgcccctctattatctgcgggaattacgtcatttagcgcgatgcgtaattcagggatcgacaaacccggtcttcatgtaggcgttgtcggtttaggggggttgggtcacctagcagtcaagtttgcaaaagctttcggcttaaaggtcactgtaattagcaccacaccgtccaagaaagatgatgcaatcaacggtcttggggccgatgggttcctgttaagccgtgacgatgagcagatgaaagccgccattggaacgctggatgccattatagacactttggcagtagtccacccgattgcgcccctactagatcttctgcgtagccagggcaaatttctgctgctaggcgccccttctcagagtttggaactacctccgattcccttgttaagtggtggcaagagcattattggtagtgctgctggaaacgtaaagcaaacacaagagatgcttgatttcgccgctgaacatgatatcacggcgaatgtggaaattatacccatagagtatataaacacggctatggaaagactagacaaaggcgacgtaagatacaggtttgtggtcgacatcgaaaataccttaacccccccttccgaactgtaa

SEQ ID NO: 17 - DNA, 963 bp, Catharanthus roseus
atgggctcaagtgacgagactatcttcgacttaccgccgtacataaaagtcttcaaagacggacgtgtagagaggctacatagtagcccctacgtgcctcctagcttgaacgatccagagaccgggggtgtgtcatggaaggatgttccgatatccagcgtggtcagtgctcgtatttacctacctaagattaataatcacgacgagaaattacctatcatagtttatttccacggagcagggttctgtctggaatcagcgtttaagtcattttttcacacttatgtcaaacacttcgtggccgaagccaaggccattgccgtcagtgttgagtttaggctggctccggagaatcacttgcccgctgcctatgaagattgttgggaagcgttacagtgggtagccagtcacgtgggactggacataagtagtttaaagacgtgtatcgacaaagatccgtggattataaattatgcagatttcgacaggctgtacttgtggggggattccacgggtgcgaatatagttcacaacactcttataagaagcggaaaagaaaagttaaatggtggtaaggtcaagattctaggtgcgatcttatattatccgtatttcttgattcgtacttctagcaagcaaagtgattacatggagaatgagtatagatcctattggaaacttgcgtatccggatgcgccgggcggaaatgataatccgatgattaatccaactgcagagaatgcgccggatctagctggatatggatgttcccgtttgttaatatcaatggtcgctgatgaggccagagacataaccttgttgtatatcgacgctcttgagaaaagcggttggaaaggggaactagatgttgcggattttgataagcagtatttcgaattgtttgagatggaaacggaggttgctaagaatatgttaagaaggttagcatcttttatcaaataa

SEQ ID NO: 18 - DNA, 993 bp, Catharanthus roseus
atgaatagcagcacggacccgaccagtgatgaaacaatctgggatctgtccccgtatattaagatcttcaaggacggaagagtagaacgtctacacaactccccatacgtgcccccgtcactaaatgatcctgagacgggggtgagttggaaggacgttcccatttccagtcaagtttcagcgagagtttacatccctaagatttccgaccatgagaagctgccgattttcgtctacgtgcacggtgcgggtttttgcctagaatcagccttcaggtccttcttccatacttttgtaaaacatttcgtcgctgaaacgaaggttatcggtgtatctatagaataccgtttggcgcccgaacaccttctgccggccgcctatgaagattgctgggaggcgttacagtgggtagcgtctcatgtaggattggataatagcggtttgaagacggctattgacaaagacccttggataataaactatggagactttgatagattatatcttgcgggggatagcccaggagccaacatcgtacacaatacacttataagggccgggaaagagaaattaaaaggaggagttaaaatacttggagctatactttactacccgtactttatcatcccaacgagcactaagttgtctgacgattttgaatataactacacatgctactggaaattggcttaccccaatgcccctggcgggatgaacaacccaatgataaaccctatagctgagaatgctcctgatcttgcggggtacggttgttctagacttttggtaaccttggtttccatgatttccactacgcccgatgaaactaaagatatcaatgcggtctatattgaggccctggagaagagtggctggaagggagagttagaagtggccgattttgacgcagactacttcgagttattcaccctagaaacagagatgggtaagaacatgtttagacgtctggccagtttcattaaacatgagtaa

SEQ ID NO: 19 - DNA, 1662 bp, Uncaria tomentosa
atgagtacgcctgctacgaagttcagtggaacagtatctcgttcagactttcccgagggttttctgttcggcagtgcttcatctgcctttcagtatgaaggggcgcacaatgtagatggaagattgccttctatctgggatacgttcctagtcgaaacccatccagatatcgtcgccgctaacgggttggatgccgttgagttttactaccgttacaaagaagatattaaggcgatgaaggacattggcttggatacatttcgtttcagcctgagctggcctaggattctgccaaatgggagacgtactcgtgggcccaacaatgaagagcagggggtgaacaaattagcaatcgatttttacaacaaggttataaaccttttgcttgagaatggaatagagccgtcagttaccttatttcactgggacgtgcctcaagctttagaaacagagtatctgggttttttatctgaaaaatctgttgaggactttgtagattatgctgacctttgtttccgtgagttcggagaccgtgtgaaatactggatgaccttcaatgagacatggtcctattctttatttggataccttcttggtactttcgcgcctggaagaggatcaactaacgaggagcaaagaaaggcaatagcggaagacctacccagctccttaggcaaatcaaggcaagcgttcgctcacagtaggaccccaagggcaggagaccctagtacggagccgtacatagtgacccacaaccaactactagcgcacgctgcggctgtgaagctttaccgttttgcataccaaaacgcccagaacgctcagaaaggaaaaataggcattggtctagtatctatttgggcagaaccccataacgacacaaccgaggacagagatgcagcacaacgtgtcttggattttatgcttggatggttgttcgatccggtggtcttcggcaggtatccagagagtatgaggcgtttgctagggaacagattaccggaatttaaaccacaccagttgagagacatgatcggttcatttgacttcatagggatgaactattataccactaattccgtcgcgaatctgccctatagtcgttctatcatctataatcccgattcacaggccatctgttatcccatgggggaagaggccgggagcagctgggtgtacatttacccagagggcttgctaaaattattactgtacgttaaagagaaatacaacaaccctctgatttacataacagagaacggcatcgatgaagttaacgatgaaaatttaaccatgtgggaagcgttgtatgatactcaaaggatcagttatcataagcagcatttggaggccactaagcaagcgatatcacaaggcgtggacgttagggggtattacgcatggtcttttaccgataatctagagtgggcaagcggtttcgattcaagatttggcctaaattatgtacatttcggtcgtaaactagaaaggtacccaaaattatccgctggttggttcaagtttttcttggaaaatgggaaaagtgcaagcttttgttggagcatcatagggaataacatttgtttgaataaaaggagccgttgtaccttagttgattgccgtatatacatattgttagttataaggatctatgtttgttaa

SEQ ID NO: 20 - DNA, 1668 bp, Catharanthus roseus
atgggcagcaaagatgatcagagtttagtagttgcgatatctccagctgctgaaccaaacggaaatcatagtgtgcccattccatttgcttaccctagcatcccaatccagccaagaaaacataataaaccaatagttcatagaagagattttccatcagacttcatcctaggagctggaggcagtgcgtatcagtgtgaaggtgcatataacgaaggtaatagaggcccatcaatttgggatactttcacaaaccgttaccctgcgaagatagcagatggcagtaatggcaatcaagccatcaactcttacaatttgtacaaggaagacattaaaataatgaaacaaaccgggcttgaaagttatagattttcaatttcttggtctagagttttaccaggaggtaaccttagcggaggcgttaataaggatggagtgaagttttatcatgacttcatcgacgaactgctggctaatggtatcaaaccatttgctacgctgtttcactgggacctaccacaggctttggaagatgagtacggtggtttcttatctgacagaattgtcgaagattttactgaatatgctgaattttgtttctgggaatttggagacaaagtaaaattctggaccacttttaacgagcctcatacttatgtagcgagcggttacgcaactggagaatttgctcctggaagagggggcgccgatggaaaaggcaacccaggtaaggaaccatacatagctactcataacttgctactttctcataaggcggcggttgaagtctacaggaaaaactttcaaaagtgtcaaggtggcgaaatcggtattgtattaaactcaatgtggatggaaccattaaacgaaaccaaggaagacatcgatgcaagagagaggggtccggatttcatgttaggttggtttatagaacctttaactactggtgaatatcctaaatctatgagggctttggtcggttctagattaccggaattttctactgaagattccgaaaaattgactggttgctacgatttcatcgggatgaattattacacgactacctacgttagcaatgctgataagatcccagacacgcccggctatgaaactgatgccagaattaataagaatatctttgtaaagaaggttgatggtaaggaagtgagaatcggggaaccatgctacggtggctggcaacacgttgttccttctggtttgtataacttgctagtgtataccaaagaaaagtatcacgtccccgtgatctatgtttccgagtgtggtgtagttgaagagaatagaaccaacatcttgctgactgaaggaaaaacaaacattcttttgactgaagccagacatgataagctaagggttgacttcctacaatcacatctggcgtccgtcagggacgcaattgatgacggtgtcaatgttaaggggtttttcgtctggtcttttttcgataatttcgagtggaatttggggtatatttgcagatatggtattatccatgttgattataaaactttccaaagatatccgaaagactcagccatttggtacaagaattttatctctgagggattcgtaaccaacactgctaaaaagaggtttagagaagaggataagttggtcgagctagttaagaagcaaaagtattaa

SEQ ID NO: 21 - DNA, 1599 bp, Camptotheca acuminata
atggaggcacaaagtattcctttaagtgttcacaacccttcctcaatccatcgtagagatttcccaccagattttatttttggtgctgccagcgccgcataccagtatgaaggggccgctaacgagtatggtaggggaccatccatatgggacttttggacccaaagacaccctggtaaaatggtcgattgctcaaatggaaatgtcgctatcgattcatatcatagattcaaagaggacgttaagataatgaaaaagattgggttagacgcataccgtttttctataagttggagcagattgcttccgtcaggcaaactgtcaggaggagtcaacaaggaaggtgtcaacttttacaatgatttcattgacgagttggtcgctaacggcatagaaccatttgtcacactttttcattgggatctgcctcaagccctggagaatgagtacggcggattcctatctcccaggataatcgccgactacgtcgacttcgcagagttatgtttctgggaatttggggatagagttaaaaattgggctacgtgtaatgagccatggacctatacggtgtcaggctatgtgttaggcaactttcctcctggcaggggtccatcaagccgtgaaacgatgaggtccttgcctgctctatgtcgtcgtagcatcctgcatacgcatatctgcacggatggaaacccggccacagaaccttacagagtagctcaccatctactactaagtcatgctgcggcggtcgagaaatataggacgaaatatcagacatgtcagagaggaaagataggcatcgtgctaaatgttacttggttagagcctttctccgagtggtgcccaaatgataggaaggcagcggagagaggcctagattttaagttaggttggttcttggagccagtcataaatggggactacccgcaaagtatgcagaacttagtgaagcaaagactgcctaagttttccgaggaggagtccaagttattaaaaggctccttcgacttcataggcatcaactattatacatccaactacgcaaaggacgcaccccaagcggggagcgacgggaagctttcttataataccgatagtaaagtcgaaataactcatgagaggaaaaaggacgttccgattggtcctcttggtgggtccaactgggtgtacttgtacccagaagggatatataggttgctggattggatgagaaaaaaatataacaacccgctggtatacataaccgagaacggggtagacgacaagaacgatacaaaattaaccctaagcgaggcacgtcatgacgagactaggcgtgactaccacgagaagcacctacgtttcctacattacgcaacccacgagggagccaacgtgaaggggtattttgcgtggtccttcatggacaacttcgaatggagcgaaggatatagtgtccgttttggcatgatatacatagactataaaaacgatttggcccgttacccaaaagactccgcaatctggtataagaatttcttgacgaagaccgaaaaaaccaaaaaaagacaattggaccacaaggagttagacaatataccccaaaagaagtaa

SEQ ID NO: 22 - DNA, 1575 bp, Glycine soja
atggctttcaaaggttactttgttctggggttgattgcgctagtagtggtgggtacctccaaagtgacgtgtgagatcgaggcggacaaagtatcaccgattatagacttcagcctgaaccgtaactcattcccagaaggtttcatcttcggagccgcttctagcagttatcagtttgaaggtgccgccaaggaagggggaagggggccgtctgtttgggacaccttcacacataaataccccgacaagatcaaggacggaagcaatggggacgttgccatagactcatatcaccattataaagaagatgttgccattatgaaagacatgaatctggattcctacagacttagcatttcatggtcaaggatcttaccggaaggcaaattaagtggggggattaaccaagagggcattaattactataataatcttatcaacgaactggtcgcaaatggcattcagcccttggttacgctgttccactgggatctacctcaagcactggaggaggaatacggcggctttttgtcacctaggatcgttaaggatttcggagattacgccgagttgtgcttcaaagagttcggagatagggtcaagtactggataacgctaaatgagccttggagttacagcatgcacggctatgcgaaaggtgggatggccccgggacgttgtagtgcgtggatgaacctgaattgcacagggggagattccgcgacagaaccctatttagtagcccatcaccagctactggcacatgcagtggcaattcgtgtttacaagaccaagtaccaggcgtcccaaaaggggtccatcggaataacgttgatagctaattggtatattccacttcgtgataccaaatccgatcaagaagctgctgagcgtgccatagatttcatgtacgggtggttcatggatccgctaaccagcggtgactaccctaagtccatgcgttccttggttcgtaagaggttacccaaattcactacagaacagacaaagcttttgattggctcttttgacttcatcggcttaaactactacagttcaacatacgttagtgacgcgcctttactttcaaacgctagacctaactatatgacggacagtttgaccacgccagcatttgaacgtgatggcaagcccattgggattaagatagcctctgaccttatctacgtgacccccaggggcatccgtgatctgcttttgtatacgaaggaaaaatataacaacccgttgatttatatcacagaaaatggtatcaacgaatacaatgagccaacatacagccttgaggagtcattgatggatatctttcgtatagattaccattatagacacctattttacttgaggagcgccataagaaacggtgcgaatgtgaagggctatcatgtatggagcttatttgacaacttcgaatggagtagcgggtacactgtgaggtttgggatgatttatgtggactacaaaaacgacatgaagcgttacaagaaacttagtgctttgtggttcaagaatttcttgaagaaagagtcccgtttatatggaacgtccaagtaa

SEQ ID NO: 23 - DNA, 1080 bp, Chatharanthus roseus
atggcagctaagtcaccagagaatgtctatcccgtgaaaaccttcggtttcgctgcgaaggattccagtggcttcttctctcccttcaatttttctcgtagggccactggcgagaacgatgtgcagtttaaagtgttgtattgcgggacctgtaattacgaccttgaaatgtcaacgaacaagtttggaatgaccaaatatccctttgtaatagggcatgagatcgtgggtgtagtaacggagataggctccaaggtccaaaagttcaaagtcggtgataaggtcggcgttggtggctttgtgggcgcctgtgaaaaatgcgaaatgtgcgttaatggcgttgaaaataactgttcaaaagttgaaagtaccgatggacacttcggtaacaactttggtggatgctgtaacataatggtagtgaatgagaagtatgcagtagtgtggccagaaaatctgcccttacacagcggtgttccccttctgtgcgctggaatcacgacatattctcccttgcgtcgttatgggttggacaaaccgggcctgaatattgggatagctggactggggggactgggacacctggctattcgtttcgcaaaagcattcggcgccaaggtcactctaataagttctagcgttaaaaagaagcgtgaagctcttgaaaaatttggggtagacagcttcctgctgaattctaaccctgaagaaatgcagggggcatatgggaccttagatgggattatcgatacaatgcccgttgcccactctattgtgccgtttttagcacttctaaaaccgttaggcaagctaattattttaggagtacctgaggagcccttcgaggtccccgcacccgccttgctgatgggtggtaagctgatcgcgggctcagctgctggaagtatgaaggagactcaagaaatgattgattttgctgctaaacataatatcgttgcggacgtggaagttatacctatagattacttaaacactgcaatggaaagaattaaaaactcagatgtcaaatacagattcgtgatagacgttgggaacactttaaaatccccttcattctaa

SEQ ID NO: 24 - protein, 532 aa, Rauvolfia serpentina
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 25 - protein, 534 aa, Gelsemium sempervirens
MATPSSTIVPDATKINRRDFPSDFVFGAASSAYQIEGGASEGGRGPSIWDTFTKRRPEMVKGGSNGNVAIDSYHLYKEDVKILKNLGLDAYRFSISWSRILPGGNLSGGINKEGIDFYNNFIDELIASGIQPYVTLFHWDVPQALEDEYGGFLSPKIVDDFRDYAELCFWNFGDRVKNWITLNEPWTFSVDGYVAGTFAPGRGATPTDQVKGPIKRHRCSGWGPQCSNSDGNPGTEPYLVTHHQILAHAAAVESYRNKFKASQEGQIGITIVAQWMEPLNEKSDSDVQAAKRALDFMYGWFMEPITSGDYPEIMKKIVGSRLPKFSAEQSRKLKGSYDFLGLNYYTANYVTSAPNPTGGIVSYDTDTQVTYHSDRNGKLIGPLAGSEWLHIYPEGIRKLLVYTKKTYNVPLIYITENGVDELNDTSLTLSEARVDPIRIKFIQDHLLQLRLAIDDGVNVKGYFVWSLLDNFEWNEGFTVRFGMIHVNYNDQYARYPKDSAIWLMNNFHKKFSGPPVKRSVEENQETDSRKRSRK

SEQ ID NO: 26 - protein, 476 aa, Scedosporium apiospermum
MSLPKDFLWGFATAAYQIEGASEKDGRGPSIWDTFCAIPGKIADGSSGAVACDSYNRAGEDIALLKELGASAYRFSISWSRIIPLGGRNDPVNQAGIDHYVKFVDDLTDAGITPFVTLFHWDLPDGLDKRYGGLLNREEFPLDFEHYARTVFKALPKVKHWITFNEPWCSAILGYNTGFFAPGHTSDRTKSAVGDSAREPWIAGHNMLVAHGRAVKAYREEFKPTNGGEIGITLNGDATYPWDPEDPEDVAACDRKIEFAISWFADPIYFGKYPDSMLAQLGDRLPTFTDEERALVQGSNDFYGMNHYTANYIKHKTDTPPEDDFLGNLETLFESKNGDCIGPETQSFWLRPNPQGFRDLLNWLSKRYGRPKIYVTENGTSIKGENDLPREQILQDDFRVEYFDSYAKAMADAYEKDGVDVRGYMAWSLLDNFEWAEGYETRFGVTFVDYANGQKRYPKKSARSLKPLFDSLIKKD

SEQ ID NO: 27 - protein, 536 aa, Rauvolfia verticillata
MESNQGEPLVVAIVPKPNASTEQKNSHLIPATRSKIVVHRRDFPQDFVFGAGGSAYQCEGAYNEGNRGPSIWDTFTQRTPAKISDGSNGNQAINCYHMYKEDIKIMKQAGLEAYRFSISWSRVLPGGRLAAGVNKDGVKFYHDFIDELLANGIKPFATLFHWDLPQALEDEYGGFLSHRIVDDFCEYAEFCFWEFGDKIKYWTTFNEPHTFTANGYALGEFAPGRGKNGKGDPATEPYLVTHNILLAHKAAVEAYRNKFQKCQEGEIGIVLNSTWMEPLNDVQADIDAHKRALDFMLGWFIEPLTTGDYPKSMREIVKGRLPRFSPEDSEKLKGCYDFVGMNYYTATYVTNAAKSNSEKLSYETDDHVDKTFDRVVDGKSVPIGAVLYGEWQHVVPWGLYKLLVYTKETYHVPVLYVTESGMVEENKTKILLSEARRDPERTDYHQKHLASVRDAIDDGVNVKGYFVWSFFDNFEWNLGFIGRYGIIHVDYNSFERCPKESAIWYKNFIAGVSTTSPAKRRREEAEGVELVKRQKT

SEQ ID NO: 28 - protein, 356 aa, Chatharanthus roseus
MAMASKSPSEEVYPVKAFGLAAKDSSGLFSPFNFSRRATGEHDVQLKVLYCGTCQYDREMSKNKFGFTSYPYVLGHEIVGEVTEVGSKVQKFKVGDKVGVASIIETCGKCEMCTNEVENYCPEAGSIDSNYGACSNIAVINENFVIRWPENLPLDSGVPLLCAGITAYSPMKRYGLDKPGKRIGIAGLGGLGHVALRFAKAFGAKVTVISSSLKKKREAFEKFGADSFLVSSNPEEMQGAAGTLDGIIDTIPGNHSLEPLLALLKPLGKLIILGAPEMPFEVPAPSLLMGGKVMAASTAGSMKEIQEMIEFAAEHNIVADVEVISIDYVNTAMERLDNSDVRYRFVIDIGNTLKSN

SEQ ID NO: 29 - protein, 501 aa, Gelsemium sempervirens
MEVMQLSFSYPALFLFVFFLFMLVKQLRRPKNLPPGPNKLPIIGNLHQLATELPHHTLKQLADKYGPIMHLQFGEVSAIIVSSAKLAKVFLGNHGLAVADRPKTMVATIMLYNSSGVTFAPYGDYWKHLRQVYAVELLSPKSVRSFSMIMDEEISLMLKRIQSNAAGQPLKVHDEMMTYLFATLCRTSIGSVCKGRDLLIDTAKDISAISAAIRIEELFPSLKILPYITGLHRQLGKLSKRLDGILEDIIAQREKMQESSTGDNDERDILGVLLKLKRSNSNDTKVRIRNDDIKAIVFELILAGTLSTAATVEWCLSELKKNPGAMKKAQDEVRQVMKGETICTNDVQKLEYIRMVIKETFRMHPPAPLLFPRECREPIQVEGYTIPEKSWLIVNYWAVGRDPELWNDPEKFEPERFRNSPVDMSGNHYELIPFGAGRRICPGISFAATNAELLLASLIYHFDWKLPAGVKELDMDELFGAGCVRKNPLHLIPKTVVPCQD

SEQ ID NO: 30 - protein, 352 aa, Chatharanthus roseus
MANFSESKSMMAVFFMFFLLLLSSSSSSSSSSPILKKIFIESPSYAPNAFTFDSTDKGFYTSVQDGRVIKYEGPNSGFTDFAYASPFWNKAFCENSTDPEKRPLCGRTYDISYDYKNSQMYIVDGHYHLCVVGKEGGYATQLATSVQGVPFKWLYAVTVDQRTGIVYFTDVSSIHDDSPEGVEEIMNTSDRTGRLMKYDPSTKETTLLLKELHVPGGAEISADGSFVVVAEFLSNRIVKYWLEGPKKGSAEFLVTIPNPGNIKRNSDGHFWVSSSEELDGGQHGRVVSRGIKFDGFGNILQVIPLPPPYEGEHFEQIQEHDGLLYIGSLFHSSVGILVYDDHDNKGNSYVSS

SEQ ID NO: 31 - protein, 714 aa, Chatharanthus roseus
MDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 32 - protein, 134 aa, Chatharanthus roseus
MASDQKLHKFDEVSKHNKTKDCWLIINGKVYDVTPFMDDHPGGDEVLLSATGKDATNDFEDVGHSDSAREMMDKYYIGEMDMATVPLKRTYIPPQQAQYNPDKTPEFVIKILQFLVPLLILGLAFAVRHYTKEK

SEQ ID NO: 33 - protein, 364 aa, Chatharanthus roseus
MAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEE

SEQ ID NO: 34 - protein, 501 aa, Chatharanthus roseus
MEFSFSSPALYIVYFLLFFVVRQLLKPKSKKKLPPGPRTLPLIGNLHQLSGPLPHRTLKNLSDKHGPLMHVKMGERSAIIVSDARMAKIVLHNNGLAVADRSVNTVASIMTYNSLGVTFAQYGDYLTKLRQIYTLELLSQKKVRSFYSCFEDELDTFVKSIKSNVGQPMVLYEKASAYLYATICRTIFGSVCKEKEKMIKIVKKTSLLSGTPLRLEDLFPSMSIFCRFSKTLNQLRGLLQEMDDILEEIIVEREKASEVSKEAKDDEDMLSVLLRHKWYNPSGAKFRITNADIKAIIFELILAATLSVADVTEWAMVEILRDPKSLKKVYEEVRGICKEKKRVTGYDVEKMEFMRLCVKESTRIHPAAPLLVPRECREDFEVDGYTVPKGAWVITNCWAVQMDPTVWPEPEKFDPERYIRNPMDFYGSNFELIPFGTGRRGCPGILYGVTNAEFMLAAMFYHFDWEIADGKKPEEIDLTEDFGAGCIMKYPLKLVPHLVND

SEQ ID NO: 35 - protein, 354 aa, Chatharanthus roseus
MADRVKTVGWAAHDSSGFLSPFQFTRRATGEEDVRLKVLYCGVCHSDLHNIKNEMGFTSYPCVPGHEVVGEVTEVGNKVKKFIIGDKVGVGLFVDSCGECEQCVNDVETYCPKLKMAYLSIDDDGTVIQGGYSKEMVIKERYVFRWPENLPLPAGTPLLGAGSTVYSPMKYYGLDKSGQHLGVVGLGGLGHLAVKFAKAFGLKVTVISTSPSKKDEAINHLGADAFLVSTDQEQTQKAMSTMDGIIDTVSAPHALMPLFSLLKPNGKLIVVGAPNKPVELDILFLVMGRKMLGTSAVGGVKETQEMIDFAAKHGIVADVEVVEMENVNNAMERLAKGDVRYRFVLDIGNATVAV

SEQ ID NO: 36 - protein, 323 aa, Chatharanthus roseus
MEKQVEIPEVELNSGHKMPIVGYGTCVPEPMPPLEELTAIFLDAIKVGYRHFDTASSYGTEEALGKAIAEAINSGLVKSREEFFISCKLWIEDADHDLILPALNQSLQILGVDYLDLYMIHMPVRVRKGAPMFNYSKEDFLPFDIQGTWKAMEECSKQGLAKSIGVSNYSVEKLTKLLETSTIPPAVNQVEMNVAWQQRKLLPFCKEKNIHITSWSPLLSYGVAWGSNAVMENPVLQQIAASKGKTVAQVALRWIYEQGASLITRTSNKDRMFENVQIFDWELSKEELDQIHEIPQRRGTLGEEFMHPEGPIKSPEELWDGDL

SEQ ID NO: 37 - protein, 421 aa, Chatharanthus roseus
MAPQMQILSEELIQPSSPTPQTLKTHKLSHLDQVLLTCHIPIILFYPNQLDSNLDRAQRSENLKRSLSTVLTQFYPLAGRININSSVDCNDSGVPFLEARVHSQLSEAIKNVAIDELNQYLPFQPYPGGEESGLKKDIPLAVKISCFECGGTAIGVCISHKIADALSLATFLNSWTATCQEETDIVQPNFDLGSHHFPPMESIPAPEFLPDENIVMKRFVFDKEKLEALKAQLASSATEVKNSSRVQIVIAVIWKQFIDVTRAKFDTKNKLVAAQAVNLRSRMNPPFPQSAMGNIATMAYAVAEEDKDFSDLVGPLKTSLAKIDDEHVKELQKGVTYLDYEAEPQELFSFSSWCRLGFYDLDFGWGKPVSVCTTTVPMKNLVYLMDTRNEDGMEAWISMAEDEMSMLSSDFLSLLDTDFSN

SEQ ID NO: 38 - protein, 529 aa, Chatharanthus roseus
MIKKVPIVLSIFCFLLLLSSSHGSIPEAFLNCISNKFSLDVSILNILHVPSNSSYDSVLKSTIQNPRFLKSPKPLAIITPVLHSHVQSAVICTKQAGLQIRIRSGGADYEGLSYRSEVPFILLDLQNLRSISVDIEDNSAWVESGATIGEFYHEIAQNSPVHAFPAGVSSSVGIGGHLSSGGFGTLLRKYGLAADNIIDAKIVDARGRILDRESMGEDLFWAIRGGGGASFGVIVSWKVKLVKVPPMVTVFILSKTYEEGGLDLLHKWQYIEHKLPEDLFLAVSIMDDSSSGNKTLMAGFMSLFLGKTEDLLKVMAENFPQLGLKKEDCLEMNWIDAAMYFSGHPIGESRSVLKNRESHLPKTCVSIKSDFIQEPQSMDALEKLWKFCREEENSPIILMLPLGGMMSKISESEIPFPYRKDVIYSMIYEIVWNCEDDESSEEYIDGLGRLEELMTPYVKQPRGSWFSTRNLYTGKNKGPGTTYSKAKEWGFRYFNNNFKKLALIKGQVDPENFFYYEQSIPPLHLQVEL

SEQ ID NO: 39 - protein, 365 aa, Chatharanthus roseus
MAGKSAEEEHPIKAYGWAVKDRTTGILSPFKFSRRATGDDDVRIKILYCGICHTDLASIKNEYEFLSYPLVPGMEIVGIATEVGKDVTKVKVGEKVALSAYLGCCGKCYSCVNELENYCPEVIIGYGTPYHDGTICYGGLSNETVANQSFVLRFPERLSPAGGAPLLSAGITSFSAMRNSGIDKPGLHVGVVGLGGLGHLAVKFAKAFGLKVTVISTTPSKKDDAINGLGADGFLLSRDDEQMKAAIGTLDAIIDTLAVVHPIAPLLDLLRSQGKFLLLGAPSQSLELPPIPLLSGGKSIIGSAAGNVKQTQEMLDFAAEHDITANVEIIPIEYINTAMERLDKGDVRYRFVVDIENTLTPPSEL

SEQ ID NO: 40 - protein, 320 aa, Chatharanthus roseus
MGSSDETIFDLPPYIKVFKDGRVERLHSSPYVPPSLNDPETGGVSWKDVPISSVVSARIYLPKINNHDEKLPIIVYFHGAGFCLESAFKSFFHTYVKHFVAEAKAIAVSVEFRLAPENHLPAAYEDCWEALQWVASHVGLDISSLKTCIDKDPWIINYADFDRLYLWGDSTGANIVHNTLIRSGKEKLNGGKVKILGAILYYPYFLIRTSSKQSDYMENEYRSYWKLAYPDAPGGNDNPMINPTAENAPDLAGYGCSRLLISMVADEARDITLLYIDALEKSGWKGELDVADFDKQYFELFEMETEVAKNMLRRLASFIK

SEQ ID NO: 41 - protein, 330 aa, Chatharanthus roseus
MNSSTDPTSDETIWDLSPYIKIFKDGRVERLHNSPYVPPSLNDPETGVSWKDVPISSQVSARVYIPKISDHEKLPIFVYVHGAGFCLESAFRSFFHTFVKHFVAETKVIGVSIEYRLAPEHLLPAAYEDCWEALQWVASHVGLDNSGLKTAIDKDPWIINYGDFDRLYLAGDSPGANIVHNTLIRAGKEKLKGGVKILGAILYYPYFIIPTSTKLSDDFEYNYTCYWKLAYPNAPGGMNNPMINPIAENAPDLAGYGCSRLLVTLVSMISTTPDETKDINAVYIEALEKSGWKGELEVADFDADYFELFTLETEMGKNMFRRLASFIKHE

SEQ ID NO: 42 - protein, 553 aa, Uncaria tomentosa
MSTPATKFSGTVSRSDFPEGFLFGSASSAFQYEGAHNVDGRLPSIWDTFLVETHPDIVAANGLDAVEFYYRYKEDIKAMKDIGLDTFRFSLSWPRILPNGRRTRGPNNEEQGVNKLAIDFYNKVINLLLENGIEPSVTLFHWDVPQALETEYLGFLSEKSVEDFVDYADLCFREFGDRVKYWMTFNETWSYSLFGYLLGTFAPGRGSTNEEQRKAIAEDLPSSLGKSRQAFAHSRTPRAGDPSTEPYIVTHNQLLAHAAAVKLYRFAYQNAQNAQKGKIGIGLVSIWAEPHNDTTEDRDAAQRVLDFMLGWLFDPVVFGRYPESMRRLLGNRLPEFKPHQLRDMIGSFDFIGMNYYTTNSVANLPYSRSIIYNPDSQAICYPMGEEAGSSWVYIYPEGLLKLLLYVKEKYNNPLIYITENGIDEVNDENLTMWEALYDTQRISYHKQHLEATKQAISQGVDVRGYYAWSFTDNLEWASGFDSRFGLNYVHFGRKLERYPKLSAGWFKFFLENGKSASFCWSIIGNNICLNKRSRCTLVDCRIYILLVIRIYVC

SEQ ID NO: 43 - protein, 555 aa, Chatharanthus roseus
MGSKDDQSLVVAISPAAEPNGNHSVPIPFAYPSIPIQPRKHNKPIVHRRDFPSDFILGAGGSAYQCEGAYNEGNRGPSIWDTFTNRYPAKIADGSNGNQAINSYNLYKEDIKIMKQTGLESYRFSISWSRVLPGGNLSGGVNKDGVKFYHDFIDELLANGIKPFATLFHWDLPQALEDEYGGFLSDRIVEDFTEYAEFCFWEFGDKVKFWTTFNEPHTYVASGYATGEFAPGRGGADGKGNPGKEPYIATHNLLLSHKAAVEVYRKNFQKCQGGEIGIVLNSMWMEPLNETKEDIDARERGPDFMLGWFIEPLTTGEYPKSMRALVGSRLPEFSTEDSEKLTGCYDFIGMNYYTTTYVSNADKIPDTPGYETDARINKNIFVKKVDGKEVRIGEPCYGGWQHVVPSGLYNLLVYTKEKYHVPVIYVSECGVVEENRTNILLTEGKTNILLTEARHDKLRVDFLQSHLASVRDAIDDGVNVKGFFVWSFFDNFEWNLGYICRYGIIHVDYKTFQRYPKDSAIWYKNFISEGFVTNTAKKRFREEDKLVELVKKQKY

SEQ ID NO: 44 - protein, 532 aa, Camptotheca acuminata
MEAQSIPLSVHNPSSIHRRDFPPDFIFGAASAAYQYEGAANEYGRGPSIWDFWTQRHPGKMVDCSNGNVAIDSYHRFKEDVKIMKKIGLDAYRFSISWSRLLPSGKLSGGVNKEGVNFYNDFIDELVANGIEPFVTLFHWDLPQALENEYGGFLSPRIIADYVDFAELCFWEFGDRVKNWATCNEPWTYTVSGYVLGNFPPGRGPSSRETMRSLPALCRRSILHTHICTDGNPATEPYRVAHHLLLSHAAAVEKYRTKYQTCQRGKIGIVLNVTWLEPFSEWCPNDRKAAERGLDFKLGWFLEPVINGDYPQSMQNLVKQRLPKFSEEESKLLKGSFDFIGINYYTSNYAKDAPQAGSDGKLSYNTDSKVEITHERKKDVPIGPLGGSNWVYLYPEGIYRLLDWMRKKYNNPLVYITENGVDDKNDTKLTLSEARHDETRRDYHEKHLRFLHYATHEGANVKGYFAWSFMDNFEWSEGYSVRFGMIYIDYKNDLARYPKDSAIWYKNFLTKTEKTKKRQLDHKELDNIPQKK

SEQ ID NO: 45 - protein, 524 aa, Glycine soja
MAFKGYFVLGLIALVVVGTSKVTCEIEADKVSPIIDFSLNRNSFPEGFIFGAASSSYQFEGAAKEGGRGPSVWDTFTHKYPDKIKDGSNGDVAIDSYHHYKEDVAIMKDMNLDSYRLSISWSRILPEGKLSGGINQEGINYYNNLINELVANGIQPLVTLFHWDLPQALEEEYGGFLSPRIVKDFGDYAELCFKEFGDRVKYWITLNEPWSYSMHGYAKGGMAPGRCSAWMNLNCTGGDSATEPYLVAHHQLLAHAVAIRVYKTKYQASQKGSIGITLIANWYIPLRDTKSDQEAAERAIDFMYGWFMDPLTSGDYPKSMRSLVRKRLPKFTTEQTKLLIGSFDFIGLNYYSSTYVSDAPLLSNARPNYMTDSLTTPAFERDGKPIGIKIASDLIYVTPRGIRDLLLYTKEKYNNPLIYITENGINEYNEPTYSLEESLMDIFRIDYHYRHLFYLRSAIRNGANVKGYHVWSLFDNFEWSSGYTVRFGMIYVDYKNDMKRYKKLSALWFKNFLKKESRLYGTSK

SEQ ID NO: 46 - protein, 359 aa, Chatharanthus roseus
MAAKSPENVYPVKTFGFAAKDSSGFFSPFNFSRRATGENDVQFKVLYCGTCNYDLEMSTNKFGMTKYPFVIGHEIVGVVTEIGSKVQKFKVGDKVGVGGFVGACEKCEMCVNGVENNCSKVESTDGHFGNNFGGCCNIMVVNEKYAVVWPENLPLHSGVPLLCAGITTYSPLRRYGLDKPGLNIGIAGLGGLGHLAIRFAKAFGAKVTLISSSVKKKREALEKFGVDSFLLNSNPEEMQGAYGTLDGIIDTMPVAHSIVPFLALLKPLGKLIILGVPEEPFEVPAPALLMGGKLIAGSAAGSMKETQEMIDFAAKHNIVADVEVIPIDYLNTAMERIKNSDVKYRFVIDVGNTLKSPSF

SEQ ID NO: 47 - protein, 530 aa, Vinca minor
MEITNHVELVKPNGFANNNNSHYINSSNTRSKIVHRREFPQDFIFGAGGSSYQCEGAFNEGNRGPSIWDTFTQRTPAKIADGSNGNQAINSYHMFKEDVKIMKQAGLEAYRLSISWSRILPGGRLAGGVNKDGVKFYHDFIDELLVNGIKPFVTLFHWDLPQALEDEYGGFLSPRIVEDYCEYAEFCFWEYGDKVKYWMTFNEPHTFSVNGYCLGEFAPGRGGVDQKGDPGIEPYIVTHNILLSHKAAVEAYRNKFQRCQEGEIGFVVNSLWMEPLNGNLQSDIDAHKRALDFMLGWFMEPLTTGDYPKSMRELVGERLPQFSPEDSEKLKGSYDFIGMNYYTATYVTNAVEPISQPLNYDTDDQVTKTFVRDGVPIGNVCYGGWQHDVPFGLHKLLVYTKETYHVPVLYVTESGVVEENKTNVLLSEARRDIHRMEYHQKHLASVRDAIDDGVNVKGYILWSFFDNFEWSLGFICRFGIIHVDFKSFERYPKESAIWYKNFIAGKSTTLPLKRRRLEAQEVESVKMQKV

SEQ ID NO: 48 - protein, 547 aa, Amsonia hubrichtii
MATIPKVIDATNISRRPFPTDASKISRRDFPSDFVFGTGTSAYQVEGAASEGGRGPSIWDTFTERRPDKVNGGTNGNMAVNSYHLYKEDVKILKNLGLDAYRFSISWSRVLPGGRLSAGINKEGINYYNNLIDELLANGIQPYVTLFHWDVPQALEDEYGGFLSSRIADDFCEYAELCFWEFGDRVKHWITLNEPWTFSVSGYATGNFPPGRGATSPEQLSHPTVPHRCSASTMPCIRSTGNPGTEPYWVTHHLLLAHAAAVESYRTKFQRGQEGEIGITVVSEWMEPLDENSESDVKAAIRALDFNLGWFMEPLTSGDYPESMKKIVGSRLPKFSDEQSKKLRRSYDFLGLNYYSATYVTNASTNTSGSNIFSYNTDIQVTYTTKRNGVLIGPLAGPHWLNIYPEGIRKLLVYTKKTYNVPLIYITENGVYEVNDTSLTLSEARVDNTRTKYIQDHLFNVRQAINDGVNVKGYFIWSLLDNFEWDQGYTIRFGIVHVNYNDNFARYPKESAIWLMNSFNKKHSKIPVKRSIQDEDQEQVSNKKSRK

SEQ ID NO: 49 - protein, 535 aa, Handroanthus impetiginosus
MNQDKMALQEYLATPTRIIRRDDFAKDFVFGSASSAYQFEGAAQEDGRGPSIWDAWTLNQPSNITDRSNGNVAIDHYHKYKEDVKLMKKTGLAAYRFSISWPRILPGGKLSGGINQEGINFYNNLIDTLLAEGIEPYVTLFHWDLPLVLQQEYGGFLSENIVKDYCEYVELCFWEFGDRVKHWITFNEPYPFCVYGYVTGTFPPGRGSSSPDNNSAICRHKGSGVPRACAEGNPGTEPYLAGHHLLLAHAYAVDLYRREFQPYQGGNIGITEVSHFFEPLNDTQEDRNAASRALDFMLGWFLAPLATGDYPQSMRNGAGDRLPKFTREQTKLIKDSYDFLGLNYYATFYAIYTPRPSNQPPSFSTDQELTTSTERNNVAIGQTVVSNGLGINPRGIYNLLVYIKEKYNVGLIYITENGMRETNDTNLTVSEARKDQVRIKYHQDHLHYLKMAIRDGVNVKAYFIWSFADNFEWADGFTIRFGIFYTDFRDGHLKRYPKSSAIWWTRFLNNKLMKSGSFKRLTQNQCEDDTDSQKK

SEQ ID NO: 50 - protein, 536 aa, Sesamum indicum
MANNGPGAQVARYVGAKLTRHDFPPDFIFGGATSAYQVEGAYAQDGRSLSNWDVFALQRPGKISDGSNGCVAIDNYYRFKEDVALMKKLGLDSYRFSIAWSRVLPGGRLSGGINREGIKFYNDLIDLLLAEGIEPCVTIFHFDVPQCLEEEYGGFLSPKIVQDFAEYAELCFFEFGDRVKFWVTQNEPVTFTKNGYVVGSFPPGHGSTSAQPSENNAVGFRCCRGVDTTCHGGDAGTEPYIVAHHLIIAHAVAVDIYRKNYQAVQGGKIGVTNMSGWFDPYSDAPADIEAATRAIDFMWGWFVAPIVTGDYPPVMRERVGNRLPTFTPEQAKLVKGSYDFIGMNYYTTYWAAYKPTPPGTPPTYVSDQELEFFTVRNGVPIGEQAGSEWLYIVPYGIRNLLVHTKNKYNDPIIYITENGVDEKNNRSATITTALKDDIRIKFHQDHLAFSKEAMDAGVRLKGYFVWALFDNYEWSEGYSVRFGMYYVDYVNGYTRYPKRSAIWFMNFLNKNILPRPKRQIEEIEDDNASAKRKKGR

SEQ ID NO: 51 - protein, 539 aa, Tabernaemontana elegans
METTHSPLVVAIAPRPNAVADMKNSNATRPASKVVHRREFPEDFIFGAGGSAYQCEGAANEGNRAPSIWDTFTQRTPGKIADRSNGDKAINSYHMYKEDVKIMKQTGLEAYRFSISWSRVLPGGRLSAGVNKEGVKFYHDFIDELLANGIKPFATLFHWDVPQALEDEYGGFLSSRIVDDFREYAEFCFWEFGDKVKNWTTFNEPHTFSVNGYTLGEFAPGRGGYDKGDPGTEPYLVSHNILLAHRTAVEIYREKFQECQEGEIGFVVNSTWMEPLHPNRADIDAQKRALDFMLGWFMEPLTTGDYPKSMRKLVGGRLPTFSPEESEGLEGCYDFIGINYYTATYVTDAVKSTSERLDYNTDGQYTTTFDRDNVPIGSVLYGGWQHVVPVGLYKLLVYTKDTYHVPVVYVTENGMVEQNKTSMLLPEARHDTNRVDFHREHIASVRDAIDDGVNVKGYFVWSFFDNFEWNLGFTCRYGIIHVDFESFARYPKDSAIWYKNFIYGKSLTLPVKRPRDEDREVELVKRQKKRELRRKIMKK

SEQ ID NO: 52 - protein, 523 aa, Vigna unguiculata
MAFYSTLFLGLFALLLVRSSKVTSHETVSVSPTIDISINRNTFPQGFIFGAGSSSYQFEGAAMEGGRGESVWDTFTHKYPAKIQDRSNGDVAIDSYHNYKEDVKMMKDVNLDSYRFSISWSRILPKGKLSGGINQEGINYYNNLINELVANGIKPFVTLFHWDLPQALEDEYGGFLSPLIVKDFRDYAELCFKEFGDRVKYWVTLNEPWSYSQNGYASGEMAPGRCSAWMNSNCTGGDSSTEPYLVTHHQLLAHAAAVRLYKAKYQTSQEGVIGITLVANWFLPLRDTKADQKAAERAIDFMYGWFMDPLTSGDYPKSMRSLVRTRLPKFTADQARQLIGSFDFIGLNYYSTTYSSDAPQLSNANPSYITDSLVTAAFERDGKPIGIKIASDWLYVYPRGIRDLLLYTKDKYNNPLIYITENGVNEYNEPSLSLEESLMDTFRIDYHYRHLYYLLSAIRNGANVKGYYVWSFFDNFEWSSGYTSRFGMVFIDYKNGLKRYPKLSAMWYKNFLKKETRLYASSK

SEQ ID NO: 53 - protein, 525 aa, Nyssa sinensis
MENSSDLLLRSSFPNDFIFGSGSSSYQYEGGANEGGKGPSIWDDYTQRFPGKMQDGSNGNVANDSYHRYKEDVAIIKKVGLNAYRISISWPRVLPTGRLSGGVNKEGIEYYNNVINELLANGIEPYVTLFHWDLPKALQDEYGGFLSSQIVVDFCNYAELCFWEFGDRVKHWVTFNESWSYSVLGYVNGTLAPGRGASSPENIRSLPAIHRCPAALLQKIIADGDPGIEPYLVAHNQLLSHAAAVQLYRQKFQVVQSGKIGITLVTTWFEPLSETSESDKKAADRAQDFKFGWFMDPLTTGDYPSSMRANVGSRLPKFSQEQSELLQGSFDFIGLNYYTASYATDAPKPDNDKLSYNTDSRVELLSDRNGVPIGPNAGSGWIYVYPQGIYKLLGYIKTKYNNPLLYVTENGISEENDATLTLSQARVDDNRKDYLEKHLLCVRDAIKEGANVKGYFMWSLMDNFEWSQGYTVRFGLIYIDYKDGVLTRYPKDSAIWFMNFLKNVIPTSRKRPLPSASPAKPAKKR

SEQ ID NO: 54 - protein, 476 aa, Lomentospora prolificans
MSLPKDFLWGFATAAYQIEGAAEKDGRGPSIWDTFCAIPGKIADGSSGAVACDSYNRTAEDIALLKDLGVTAYRFSISWSRIIPLGGRNDPINQAGIDHYVKFVDDLTDAGITPFVTLFHWDLPDGLDKRYGGLLNREEFPLDFEHYARTMFKALPKVKHWITFNEPWCSAILGYNTGFFAPGHTSDRSKSAVGDSAREPWIAGHNMLVAHGRAVKTYREDFKPTNGGEIGITLNGDATYPWDPEDPEDVAACDRKIEFAISWFADPIYFGKYPDSMLAQLGDRLPTFTDEERALVQGSNDFYGMNHYTANYIKHKTGTPPEDDFLGNLETLFDSKNGECIGPETQSFWLRPNPQGFRDLLNWLSKRYGYPKIYVTENGTSLKGENDMERDQILEDDFRVAYFDGYVRAMAEASEKDGVNVRGYLAWSLLDNFEWAEGYETRFGVTYVDYENGQKRYPKKSAKSLKPLFDSLIKTD

SEQ ID NO: 55 - protein, 500 aa, Actinidia chinensis var. chinensis
MRKGIVLAVVLVVLRVQTCIAQINRASFPKGFVFGTASSAYQYEGAVKEDGRGQTVWDEFAHSFGKVLDFSNADIAVNQYHLFDEDIKLMKDMGMDAYRFSIAWSRIFPNGTGEINQAGVDHYNNLINALLANGIEPYVTLYHWDLPQALEDRYNGWLHPQIIKDFALYVETCFEKFGDRVKHWITFNEPHTFTIQGYDVGLQAPGRCSILLHIFCRGGNSAIEPYIIAHNVLLSHATVVDIYRRKYKPKQHGSVGVSFDVIWFEPATNSTVDIEAAQRAQDFQLGWFIEPLIFGEYPSSMITRVGSRLPRFTKAESALLKGSLDFIGINHYTTFYAKPNTSNIIGVLLNDSIADSGAITLPFRDGTPIGDRANSIWLYIVPHGIRSLMNYIKQKYGNPPVIITENGMDDANSPLISLKDALKDEKRIKYHNDYLESLLASIKDDGCNVKGYFVWSLLDNWEWAAGFSSRFGLYFVDYGDKLKRYPKDSVKWFKNFLTSA

SEQ ID NO: 56 - protein, 493 aa, Heliocybe sulcata
MAQKLPSDFLWGMATASYQIEGSPDADGRGPSIWDTFSHLPGKTLDGLTGDIATDSYRLRDQDIALLKQYGVKSYRFSISWSRVIPLGGRNDPINEKGIKWYSDLIDELLEAGIVPFVTLYHWDLPQALHDRYGGWLNKDEIVADFVNYARLCFERFGDRVKYWLTFNEPWCISILGYGRGVFAPGRSSDRTRSPEGDSRTEPWIVGHSVIVAHASAVKLYRDEFKSRQHGVIGITLNGDMALPWDDSEECRQAAQHALDVAIGWFADPVYLGHYPPFMRQFLGDRLPTFTPEEEKLVKGSSDFYGMNTYTTNLIRPGGDDEFQGNVQYTFTRPDGSQLGTQAHCAWLQTYPEGFRALLNYLWNRYHMPIYVTENGFAVKNENNMPLEQALKDTDRIEYFKGNCEALVKAVHEDGVDLRGYFPWSFLDNFEWADGYQTRFGVTYVDYATQKRYPKESAWFLVNWFKENVNSPKSSGEPRTSRIPNGAVPNGHI

SEQ ID NO: 57 - protein, 469 aa, Moniliophthora roreri MCA 2997
MKLPKDFLFGYATASYQIEGSSDVDGRGPSIWDTFSHTPGKIVDGTNGDVATDSYQRWKDDVKIVKDYGANAYRFSISWSRIIPLGGKDDPVNPEGIRFYRTLIEELLNNGITPCVTLYHWDLPQALHDRYGGWLDRRVIEDFVRYCEICFEAFGNSVKHWITFNEPWCISCLGYGYGVFAPGRSSNRNRSEAGDSTREPWIVAHNLLLAHASAVASYRQKFWPSQAGSIGITLDCVWYMPYDESNAEDVDAAQRALDTRLGWFADPIYKGHYPTSLKAMLGNRLPEFTTEEQALIKGSSDFFGLNTYTSNLVQPGGSDEFNGKVKTTHTRADGSQLGKQAHVPWLQAYPPGFRALLNYLWKTYGKPIYVTENGFAIKDENRLPPEDAIHDQDRVDYYRGYTNALAHAANEDGVDVKAYFAWSLLDNFEWAEGYQVRFGVTFVDFETQQRYPKDSSKFLAEWYRSSLAK

SEQ ID NO: 58 - protein, 492 aa, Rauvolfia serpentina
MSLPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 59 - protein, 476 aa, Pyricularia grisea
MSLPKDFLWGFATASYQIEGAIDKDGRGPSIWDTFTAIPGKVADGSSGVTACDSYNRTQEDIDLLKSVGAQSYRFSISWSRIIPIGGRNDPINQKGIDHYVKFVDDLLEAGITPLITLFHWDLPDGLDKRYGGLLNREEFPLDFEHYARVMFKAIPKCKHWITFNEPWCSSILAYSVGQFAPGRCSDRSKSPVGDSSREPWIVGHNLLVAHGRAVKVYREEFKAQDKGEIGITLNGDATFPWDPEDPRDVDAANRKIEFAISWFADPIYFGEYPVSMRKQLGDRLPTFTEEEKALVKGSNDFYGMNCYTANYIRHKEGEPAEDDYLGNLEQLFYNKAGECIGPETQSPWLRPNAQGFRELLVWLSKRYNYPKILVTENGTSVKGENDMPLEKILEDDFRVQYYDDYVKALAKAYSEDGVNVRGYSAWSLMDNFEWAEGYETRFGVTFVDYENGQKRYPKKSAKAMKPLFDSLIEKD

SEQ ID NO: 60 - protein, 534 aa, Ophiorrhiza pumila
MEFLNPAFTRVPSGFLRRKDFGSDFIFGSATSAFQVEGGMREDGRGPSIWDSFAEKRNLFAPYSEDAINHHKNYEEDVKLMKEIGFDAYRFSISWTRILPTGKKESRNQKGIDFYKKLLKNLKIKGIEPYVTLLHFDPPQNLEDKYYGFLNRQIADDFCDYADICFKEFGNDVKHWITINEPWSFAYGGYFTGNLAPGYHAQTDKIAPHQSTKIPNDDDDDAHHKSSIFPPSRFSLPPSSSSASETPAIIPAKKLPYPDVNKYPYLVAHHQILAHAKAVKLYRQNYQRTQKGKIGIVLVSQWYISLDDDPDNKEATQRANDFMLGWFLDPIFSGDYPASMRKYVTKGYLPEFSSADKEMIKGSFDFLGLNYYTARYVTYEETGGGNYVLDQRARFHVKRKGKLIGDEKGASGWIYGYPRGMLDLLVYMKEKYNKPTIYITETGIDDPDDDSSTHWKSFYDQDRIMFYHDHLSYIKQAMRKGVNVKGFFAWSLMDNFEWDVGFKSRFGITYIDFEDGSKRCPKLSASWFKYFLEN

SEQ ID NO: 61 - protein, 470 aa, Hydnomerulius pinastri MD-312
MTEAKLPKDFTWGFATASYQIEGAYNEGGRADSIWDTFTRLPGKIADGSSGEVATDSYHRWKEDVALLKSYGVNSYRFSLSWSRIIPLGGREDKVNAEGVAFYRNFAQELVKNGITPYMTLYHWDLPQALHDRYGGWLNKEEIVKDYVNYAKVCYESFGDIVKHWITHNEPWCVSVLGYGKGVFAPGHTSDRAKFHVGDSSTEPYIVAHSMLLAHGYAVKLYREQFQPQQKGTIGITLDSSWFEPLTNTQENADVAQRAFDVRLGWFAHPIYLGYYPEALKKQCGSRLPEFTAEEIAVVKGSSDFFGLNHYTTHLVSEGGDDEFNGYAKQTHKRVDGTDIGTQADVNWLQTYGPGFRKLLGYIYKKYGKPIIITESGFAVKGENSKTIEEAINDTDREEYYRDYTKAMLEAVTEDGVDVKGYFAWSLLDNFEWAEGYRIRFGVTYVDYKTQKRYPKHSSKFLKEWFAAHI

SEQ ID NO: 62 - protein, 556 aa, Helianthus annuus
MATFDLTDQIAPFPDEISSADFDSDFVWGAATSAYQIEGAACEGGKGPSIWDVFCLTDPGRIVGGDNGNIAVNSYYKTKEDVQTMKKMGLQAYRFSLSWSRILPGGKLKLGINQEGVDYYNNLINELLANDIEPYVTLWHWDTPNVLEAEYGGFLCEKIVYDFVNYVEFCFWEFGDRVKHWTTLNEPHSYVEKGYTTGKFAPGRGGEGMPGNPGTEPYIVGHYLLLSHAKAVDLYRRRFQASQGGTIGITLNTKFYEPLNSELQDDIDAALRAIDFMLGWFMEPLFSGKYPDTMIENVTDDRLPTFTKEQSELVKGSYDFLGLNYYASQYATTAPETNVVSLLTDSKVLEQPDNMNGIPIGIKAGLDWLYSYPPGFYKLLVYIKDTYGDPLIYITENGWVDKTDNTKTVEEARVDLERMDYHNKHLQNLRYAISAGVRVKGYFVWSLMDNFEWDEGYSARFGLIYIDFKGGKYTRYPKNSAIWYKHFLGYSNKQKTEKKKNLARERTCKSSEKTTKFELELENNCYCLDLLSFLLPRINMKVNYKFGGVKLKDEQR

SEQ ID NO: 63 - protein, 505 aa, Actinidia chinensis var. chinensis
MAINRALLILFCFLAISNTEATSKKYPPLGRSSFPKDFVFGAGSAAYQFEGGAFIDGKGDSIWDTFTHQHPEKIADRSNGTIADDMYHRYKGDVALMKTTGLDGFRFSISWSRVLPKGRVSGGVNALGVKYYNNLINEILANGMVPYVTIFHWDLPQALEDEYTGFRNKKIVDDFRDYAEFLFKTFGDRVKHWFTLNEPYTYSYFGYGTGTMAPGRCSNYVGTCTEGDSSTEPYIVTHHLILAHGAAVKLYREKYKPYQRGQIGVTLVTAWFVPTTATTTSERAARRALDFMFGWFLHPMTYGDYPMTLRALAGNRVPKFTAEETAMLQKSYDFLGVNYYTAFFASNVMFSNSINISMTTDNHANLTSVKDDGVAIGQSTALNWLYVYPKGMEDLMLYLKDNYGNPPIYITENGIAEANNDKLPVKEALKDNDRIEYLYSHLLYLSKAIKAGVNVKGYFMWAFMDDFEWDAGFTVRFGMYYIDYKDGLKRYPKYSAYWYKKFLQT

SEQ ID NO: 64 - protein, 564 aa, Handroanthus impetiginosus
MENGSGAVVAVGNPQSAGSPNAVPPDQDNSNINRDDFPNDFVFGSGTSAFQVEGAAALDGKAPSVWDDFTLRTPGRIADGSNGIVAADMYHKYKEDIRNMKKMGFDVYRFSISWPRILPGGRCSAGINRLGIDYYNDLINTIIAHGMKPFVTLFHWDLPDILEKEYNGFLSRKILDDFLEYAELCFWEFGDRVKFWTTINEPWSVAVNGYVRGTFPPSKASCPPDRVLKKIPPHRSVQHSSATVPTTRQYSDIKYDKSDPAKDPYTVGRNLLLIHAKVVCLYRTKFQGHQRGQIGIVLNSNWFVPKDPDSEADQKAAKRGVDFMLGWFLHPVLYGSYPKNMVDFVPAENLAPFSERESDLLKGSADYIGLNFYTALYAENDPNPEGVGYDADQRVVFSFDKDGVPIGPPTGSSWLHVCPWAIYDHLVYLKKTYGDAPPIYITENGMSDKNDPKKTAKQACCDSMRVKYHQDHLANILKAMNDVQVDVRGYIIWSWCDNFEWAEGYTVRFGITCIDYLNHQTRYAKNSALWFCKFLKSKKSQIQSSNKRQIENNSENVLAKRYKV

SEQ ID NO: 65 - protein, 543 aa, Carapichea ipecacuanha
MSSVLPTPVLPTPGRNINRGHFPDDFIFGAGTSSYQIEGAAREGGRGPSIWDTFTHTHPELIQDGSNGDTAINSYNLYKEDIKIVKLMGLDAYRFSISWPRILPGGSINAGINQEGIKYYNNLIDELLANDIVPYVTLFHWDVPQALQDQYDGFLSDKIVDDFRDFAELCFWEFGDRVKNWITINEPESYSNFFGVAYDTPPKAHALKASRLLVPTTVARPSKPVRVFASTADPGTTTADQVYKVGHNLLLAHAAAIQVYRDKFQNTQEGTFGMALVTQWMKPLNENNPADVEAASRAFDFKFGWFMQPLITGEYPKSMRQLLGPRLREFTPDQKKLLIGSYDYVGVNYYTATYVSSAQPPHDKKKAVFHTDGNFYTTDSKDGVLIGPLAGPAWLNIVPEGIYHVLQDIKENYEDPVIYITENGVYEVNDTAKTLSEARVDTTRLHYLQDHLSKVLEARHQGVRVQGYLVWSLMDNWELRAGYTSRFGLIHIDYYNNFARYPKDSAIWFRNAFHKRLRIHVNKARPQEDDGAFDTPRKRLRKY

SEQ ID NO: 66 - protein, 555 aa, Lactuca sativa
METTTQNTGAKFSLFQNLVHSNDFKPDFVWGAATSAYQIEGAASKGGRGESIWDVFCHNNPDAIVNGDNGNNGTNAYFKYKEDVQMMKKMGLNAYRFSISWTRIFPGGRPSNGINKEGIDYYNNLINELILCGITPYVTLFHWDTPETLEEEYMGFLSEKIIYDFTSYAGFCFWEFGDRVKNWITINEPHSYASCGYADGTFPPGRGKDGVGDPGTEPYIVAKNLLLSHASVVNLYRQKFQKKQGGKIGITLNAVFCEPLNPEKQEDKDAALRAIDFMFGWFMEPLFSGKYPDNMIKYVTGDRLPEFTAEEAKSIKGSYDFLGLNYYTSYYATSAKPSQVPSYVTDSNVHQQAEGLDGKPIGPQGGSDWLYSYPLGFYKILQHIKHTYGDPLIFITENGWPDKNNDTIGIGAACVDTQRIDYHNAHLQKLRDAVRDGVRVEGYFVWSLMDNFEWIAGYSIRFGLLYVDYNDGKYTRYPKNSAIWYMNFLKSPKKLGEQKKIPKCVPNKPIAKTQSTETSTKTSRVLAEVVLIMILSILCIVMFIFDYKMKIGCIY

SEQ ID NO: 67 - protein, 536 aa, Coffea arabica
MAAKSNVTNDLSRADFGEDFIFGSASAAYQMEGAAEEGGRGPSIWDKFTEQRPDKVVDGSNGNVAIDQYHRYKEDVQMMKKIGLDAYRFSISWSRVLPGGRLNAGVNKEGIQYYNNLIDELLANGIKPFVTLFHWDVPQTLEDEYGGFLCRRIVDDFREFAELCFWEFGDRVKHWITLNEPWTFAYNGYTTGGHAPGRGISTAEHIKDGNTGHRCNHLFSGIPVDGNPGTEPYLVAHHLLLAHAEAVKVYRETFKGQEGKIGITLVSQWWEPLNDTPQDKEAVERAADFMFGWFMSPITYGDYPKRMRDIVKSRLPKFSKEESQNLKGSFDFLGLNYYTSIYASDASGTKSELLSYVNDQQVKTQTVGPDGKTDIGPRAGSAWLYIYPLGIYKLLQYVKTHYNSPLIYITENGVDEVNDPGLTVSEARIDKTRIKYHHDHLAYVKQAMDVDKVNVKGYFIWSLLDNFEWSEGYTARFGIIHVNFKDRNARYPKKSALWFMNFLAKSNLSPTKTTKRALDNGGLADLENPKKKILKT

SEQ ID NO: 68 - DNA, 1593 bp, Vinca minor
atggaaattacaaatcacgttgaactagtcaagccgaatggctttgcaaataacaataacagccactatataaattctagtaatactagatcaaaaattgttcatagaagagaatttccacaagatttcatatttggggcaggcggttcctcgtatcaatgtgagggtgctttcaacgaaggtaatagaggaccatcaatttgggatacgttcactcaaagaaccccagctaagattgctgacggttcgaatggaaatcaagctatcaactcctatcacatgtttaaggaagatgtcaagattatgaaacaggctggtttggaggcttacagattatctatatcatggtcgagaatattaccagggggtagattagcgggtggtgtaaacaaagatggtgttaagttttatcatgatttcattgatgagctactggtaaatggtattaagccattcgtcaccttattccactgggacttgccacaagcattggaagatgaatacggtggtttcttaagtcctagaatcgtagaagactactgtgaatatgctgaattttgtttttgggaatatggtgataaggtgaagtattggatgacctttaacgagccacacaccttctcagttaatggttactgccttggtgaattcgcccctggtaggggaggagtcgaccaaaaaggcgaccctggtatcgaaccctatattgttactcacaacatcctactttcacataaggctgcggttgaagcttacagaaataaatttcagagatgtcaggaaggcgaaatcggattcgttgttaattctttatggatggagccactaaatggtaatcttcaatctgacatcgatgctcataaaagagcgctagactttatgcttggttggttcatggagccgttgaccacaggtgactatcctaaatctatgagagaactagtaggtgaaagacttccccaattctcccctgaggatagtgaaaagctaaaaggcagttatgattttataggtatgaattactatacagccacttatgttactaacgccgttgaaccaattagccaacctctgaattatgatacagacgaccaagtgaccaagacgtttgtgagagatggagttccaatcggaaatgtgtgttatggtggctggcaacatgatgtcccattcggtcttcataaactacttgtgtataccaaggaaacgtaccacgtaccagttttatacgtcacagagtcaggtgttgtagaagaaaacaagacgaatgtgcttttatccgaggctagacgtgatatccataggatggaataccatcaaaagcacttggcatctgttagagacgccattgatgacggggtcaatgttaaaggttatattttatggagtttttttgataatttcgagtggagtctaggcttcatatgtagatttggtattatccatgttgacttcaaatcgttcgaaaggtacccaaaagagtcggctatttggtacaagaattttatagccggaaaatccacaacattgccacttaaacgtaggagactagaagcacaagaagtggaatctgtgaagatgcaaaaagtctaa

SEQ ID NO: 69 - DNA, 1644 bp, Amsonia hubrichtii
atggctactattccaaaagttatcgatgctactaatatatcgagaaggcctttccccacggatgcgtcaaagatcagtagaagagattttccttcagatttcgtatttgggacaggtacctccgcatatcaggtggagggtgcggcatcagaaggaggtaggggtccaagtatctgggacacattcaccgagaggagacctgataaggtcaacggcggaactaacggaaatatggctgtgaacagttaccatttatataaggaggatgtgaaaatactaaaaaatttaggcctagacgcatatcgtttttctatatcatggtccagagtcttgcctggtggcagattgagcgcaggtatcaataaggaaggtattaattactacaacaatctaattgatgaattgttagcaaatgggatccaaccttacgttacgttattccattgggacgttcctcaagccctggaagacgaatacggcggtttcttgtcatcaagaattgccgatgatttctgcgaatacgcggaactatgtttttgggaattcggagatagagtaaagcattggattacattaaacgaaccatggaccttctctgtctctggctacgcgactggcaactttcccccaggtagaggagcaacctcacctgagcagttatcacatccaacagttcctcatagatgtagtgcttctacaatgccttgtatccgtagtacaggaaatccaggtacagaaccatactgggtcacacaccatctattgttagctcatgccgcagccgttgaatcgtatagaaccaaattccaacgtggtcaagaaggagaaataggtattacagtggtttcagaatggatggaaccactagatgaaaacagtgaatctgatgttaaagctgccattcgtgcgttggactttaatttaggatggtttatggaacctttgacatctggagattaccctgaatctatgaaaaaaatagtcggaagtagattacctaagtttagcgatgagcaaagcaagaaattaagaagatcctatgattttcttggtttaaattactattctgcaacttatgtaactaacgcttctactaacacctctggaagtaatatattttcctacaacaccgatatccaagttacttacacaactaaaagaaacggggtcttaattggtccgctagccggtccacattggttgaacatatatcccgaaggaattcgtaaattgttagtatacacaaaaaagacttataacgtgccattgatttatatcacggaaaatggagtctacgaagtcaatgatacgtctttgacgttgtcagaggctagagtcgacaatacgagaacaaaatatatccaggatcatcttttcaatgtaaggcaggcaattaatgatggagtcaacgtcaaaggatattttatatggagtcttttggataatttcgaatgggatcaaggttatacaattcgttttggcattgtccatgttaactacaatgataacttcgcacgttaccctaaagaaagcgcaatctggttaatgaattcttttaacaaaaagcatagcaagattccagttaagagatccattcaagatgaggatcaagaacaggtgagtaacaagaaatccagaaagtaa

SEQ ID NO: 70 - DNA, 1608 bp, Handroanthus impetiginosus
atgaatcaagataaaatggccctgcaagaatacctggccactccaactagaatcattagacgtgacgatttcgctaaagatttcgtttttggatctgcctcttccgcttatcaatttgaaggcgctgcgcaagaggatggtagaggtccctcgatttgggatgcctggacattgaaccaaccatcgaatataaccgatcgtagcaacggtaatgttgcaattgatcattatcataaatataaagaggatgtcaaacttatgaagaagactggcttagcggcttacagattttccatctcgtggccacgtattctaccaggtggtaagcttagtggtgggataaatcaagagggtataaatttttataataatttaatcgatactttgttggcagagggaattgaaccatatgtcaccttattccattgggatttaccacttgttttacaacaagaatatgggggtttcttaagcgagaacatagttaaagactattgtgaatacgtggaattatgcttctgggaattcggcgatcgtgttaaacattggatcacctttaatgaaccttacccattctgtgtctacggatatgtaacaggtacatttccaccgggtcgtggatcttcaagccctgataataactccgccatttgcagacacaagggtagcggagtcccaagagcctgtgccgagggtaacccaggcacagaaccctacttagctggccatcatctgttgttagctcatgcgtatgccgttgatttgtacaggagagaatttcagccatatcaaggaggcaatattggaataacagaagttagtcactttttcgaaccgttgaatgatacgcaagaagataggaacgctgcctcacgtgcgctagattttatgcttggttggtttttggcccccttggcaacaggtgattatccacagtctatgaggaacggggctggagataggttaccaaagtttactagagaacagacgaaattaattaaagatagttacgattttctaggtctgaactattatgctacattttatgccatttacacgcctagaccaagtaaccagcccccatcgtttagtacggaccaagaattgactacctcaaccgaacgtaataacgttgctatagggcagactgtcgtgagcaatggattaggaatcaaccctagaggaatctataacttactggtgtacatcaaggaaaaatataatgtcggcttgatttatatcaccgagaacggcatgcgtgaaacgaacgacactaacttaactgtttcagaagcaagaaaggatcaagttcgtattaagtatcaccaggaccatctgcattatttaaagatggctatcagagatggagtaaacgtcaaagcttattttatatggtcattcgcagacaattttgaatgggctgacggtttcacaattcgttttggaatcttttatacagactttcgtgatggacacctaaaaagataccctaaatcgtcggctatttggtggactagatttttaaataacaaattaatgaagtcagggtcttttaagagattgactcaaaatcagtgtgaggatgatacagattctcagaaaaaataa

SEQ ID NO: 71 - DNA, 1611 bp, Sesamum indicum
atggctaataatggtccaggtgctcaagttgctagatatgttggtgctaaattgactagacatgattttccaccagattttatttttggtggtgctacttctgcttatcaagttgaaggtgcttatgctcaagatggtagatctttgtctaattgggatgtttttgctttgcaaagaccaggtaaaatttctgatggttctaatggttgtgttgctattgataattattatagatttaaagaagatgttgctttgatgaaaaaattgggtttggattcttatagattttctattgcttggtctagagttttgccaggtggtagattgtctggtggtattaatagagaaggtattaaattttataatgatttgattgatttgttgttggctgaaggtattgaaccatgtgttactatttttcattttgatgttccacaatgtttggaagaagaatatggtggttttttgtctccaaaaattgttcaagattttgctgaatatgctgaattgtgtttttttgaatttggtgatagagttaaattttgggttactcaaaatgaaccagttacttttactaaaaatggttatgttgttggttcttttccaccaggtcatggttctacttctgctcaaccatctgaaaataatgctgttggttttagatgttgtagaggtgttgatactacttgtcatggtggtgatgctggtactgaaccatatattgttgctcatcatttgattattgctcatgctgttgctgttgatatttatagaaaaaattatcaagctgttcaaggtggtaaaattggtgttactaatatgtctggttggtttgatccatattctgatgctccagctgatattgaagctgctactagagctattgattttatgtggggttggtttgttgctccaattgttactggtgattatccaccagttatgagagaaagagttggtaatagattgccaacttttactccagaacaagctaaattggttaaaggttcttatgattttattggtatgaattattatactacttattgggctgcttataaaccaactccaccaggtactccaccaacttatgtttctgatcaagaattggaattttttactgttagaaatggtgttccaattggtgaacaagctggttctgaatggttgtatattgttccatatggtattagaaatttgttggttcatactaaaaataaatataatgatccaattatttatattactgaaaatggtgttgatgaaaaaaataatagatctgctactattactactgctttgaaagatgatattagaattaaatttcatcaagatcatttggctttttctaaagaagctatggatgctggtgttagattgaaaggttattttgtttgggctttgtttgataattatgaatggtctgaaggttattctgttagatttggtatgtattatgttgattatgttaatggttatactagatatccaaaaagatctgctatttggtttatgaattttttgaataaaaatattttgccaagaccaaaaagacaaattgaagaaattgaagatgataatgcttctgctaaaagaaaaaaaggtagataa

SEQ ID NO: 72 - DNA, 1620 bp, Tabernaemontana elegans
atggaaacaactcatagtccattagtggtcgctattgcaccaagaccaaatgcggtcgctgacatgaagaactctaacgctaccagaccggcatccaaggttgtgcatagaagggagttcccagaggattttatatttggagcaggtggtagtgcctaccagtgcgagggcgcagctaacgaaggaaacagggcgcctagtatctgggatacatttactcagagaacccccggtaagatcgctgataggtctaacggcgataaagccatcaactcttatcacatgtataaagaagatgtaaagattatgaagcagactgggttggaagcctacaggttttccatctcctggtccagagttcttcctggcggaaggttgagtgcaggtgtcaacaaagaaggagtcaaattttaccacgacttcattgacgagttattggcgaatggtatcaaaccttttgcaacgttgtttcactgggacgttcctcaggctttagaggacgagtatggcggattcttgtccagtcgtattgtcgacgacttcagagagtacgcggagttctgcttctgggaatttggcgataaggtaaagaattggaccacatttaatgagccacacacttttagcgtaaacgggtatactttgggagagtttgcaccaggtaggggtggatacgacaaaggtgaccctggtacagagccttacttggttagtcacaacatcttgctagcgcatcgtacagcggttgagatatatagggagaagtttcaggagtgtcaggaaggcgagatcggtttcgtcgtcaatagcacctggatggagcccctacaccctaatcgtgctgacatagatgcacaaaagagagccctagacttcatgttaggctggttcatggagcccttaacaactggcgactatccaaagagtatgcgtaagttagttggcggtcgtttaccaacgtttagcccagaagagagcgaagggcttgagggatgttatgacttcataggcataaactactatactgcaacatacgtgactgacgcggtaaagtctacgagcgaaaggctggattataacacggatggacagtatactactacgttcgacagagacaatgttcctatcggctcggtcttatacggtggttggcagcacgttgttccagttgggctatacaagttactagtctatacgaaggatacctaccacgttcctgttgtctacgtgacagagaatggcatggtagagcagaataagacatcgatgctgttgccagaggcaagacacgacaccaacagagtagattttcatcgtgagcatatcgcatctgttagggacgcaatagatgatggagttaatgttaagggatacttcgtctggtcattctttgacaacttcgaatggaacttgggattcacttgcagatacggaatcattcatgtagacttcgagtctttcgccagatatcctaaagactcagccatctggtacaagaactttatatacggcaaaagcctgacattacccgtaaagaggcccagagacgaggaccgtgaggtggagttagtcaagaggcaaaagaagagagaattacgtaggaagatcatgaagaagtag

SEQ ID NO: 73 - DNA, 1572 bp, Vigna unguiculata
atggcgttctactcgacacttttcttaggacttttcgcccttctactagtccgtagtagtaaggtgacatcacacgagaccgtgagtgtcagtcccaccatagacatatccataaaccgtaacacgttcccccagggattcatattcggcgcaggatcctcaagttaccagttcgagggtgccgccatggaaggcggcaggggcgagtcagtatgggacacattcacgcacaagtaccccgcaaagatccaggaccgttccaacggagacgtggccatcgactcataccacaactacaaagaggacgtcaagatgatgaaggacgtgaacctagactcatacaggttctcgatatcgtggagtaggatcctgcccaaggggaagctgtcaggtggaataaaccaggaaggcatcaactactacaacaacttaatcaacgagcttgtggcaaacggaataaagcctttcgtgacacttttccactgggacttacctcaggcactagaggacgagtacggcgggttcttaagccccttaatagtaaaggacttcagggactacgcagagctatgcttcaaggagttcggcgacagggtgaagtactgggtgaccttaaacgagccctggtcgtacagtcagaacggatacgcctcaggggagatggcgccgggccgttgcagcgcatggatgaacagcaactgcacaggcggcgactcatcgaccgagccttaccttgtgacacaccaccagctgttagcccacgcggccgcagtcaggctatacaaggcaaagtaccagacaagtcaggaaggcgtgatcggaatcacgttagtggcaaactggttcctacctctacgtgacacgaaggccgaccagaaggcagccgagcgtgcaatcgacttcatgtacgggtggttcatggaccctttaacaagtggcgactaccccaagtccatgcgttccttagtccgtacacgtctacctaagttcacggcggaccaggcaaggcagcttatagggagcttcgacttcataggattaaactactacagcacaacatactcaagcgacgcccctcagttatcaaacgcaaacccttcctacataacagactcattagtcaccgcagcattcgagcgtgacgggaagcctatcggcatcaagatcgcaagcgactggttatacgtataccctaggggaatacgtgacttactattatacaccaaggacaagtacaacaaccctttaatctacataacagagaacggagtaaacgagtacaacgagccgtcattatccttagaggagtcactgatggacaccttccgtatagactaccactaccgtcacctttactacctgttatcagcaatcaggaacggcgcaaacgtcaagggctactacgtatggtcattcttcgacaacttcgagtggtcatccgggtacacatcacgtttcggaatggtattcatagactacaagaacggcctgaagaggtaccccaagctttccgcaatgtggtacaagaacttcttaaagaaggagacaaggctatacgcgtcctcaaagtag

SEQ ID NO: 74 - DNA, 1578 bp, Nyssa sinensis
atggaaaattcttctgatttgttgttgagatcttcttttccaaatgattttatttttggttctggttcttcttcttatcaatatgaaggtggtgctaatgaaggtggtaaaggtccatctatttgggatgattatactcaaagatttccaggtaaaatgcaagatggttctaatggtaatgttgctaatgattcttatcatagatataaagaagatgttgctattattaaaaaagttggtttgaatgcttatagaatttctatttcttggccaagagttttgccaactggtagattgtctggtggtgttaataaagaaggtattgaatattataataatgttattaatgaattgttggctaatggtattgaaccatatgttactttgtttcattgggatttgccaaaagctttgcaagatgaatatggtggttttttgtcttctcaaattgttgttgatttttgtaattatgctgaattgtgtttttgggaatttggtgatagagttaaacattgggttacttttaatgaatcttggtcttattctgttttgggttatgttaatggtactttggctccaggtagaggtgcttcttctccagaaaatattagatctttgccagctattcatagatgtccagctgctttgttgcaaaaaattattgctgatggtgatccaggtattgaaccatatttggttgctcataatcaattgttgtctcatgctgctgctgttcaattgtatagacaaaaatttcaagttgttcaatctggtaaaattggtattactttggttactacttggtttgaaccattgtctgaaacttctgaatctgataaaaaagctgctgatagagctcaagattttaaatttggttggtttatggatccattgactactggtgattatccatcttctatgagagctaatgttggttctagattgccaaaattttctcaagaacaatctgaattgttgcaaggttcttttgattttattggtttgaattattatactgcttcttatgctactgatgctccaaaaccagataatgataaattgtcttataatactgattctagagttgaattgttgtctgatagaaatggtgttccaattggtccaaatgctggttctggttggatttatgtttatccacaaggtatttataaattgttgggttatattaaaactaaatataataatccattgttgtatgttactgaaaatggtatttctgaagaaaatgatgctactttgactttgtctcaagctagagttgatgataatagaaaagattatttggaaaaacatttgttgtgtgttagagatgctattaaagaaggtgctaatgttaaaggttattttatgtggtctttgatggataattttgaatggtctcaaggttatactgttagatttggtttgatttatattgattataaagatggtgttttgactagatatccaaaagattctgctatttggtttatgaattttttgaaaaatgttattccaacttctagaaaaagaccattgccatctgcttctccagctaaaccagctaaaaaaagataa

SEQ ID NO: 75 - DNA, 1431 bp, Lomentospora prolificans
atgtccctgccaaaggattttctatggggcttcgcaactgctgcttatcaaattgaaggtgctgcagaaaaagatggtaggggtcctagcatttgggatacattttgtgcaattccaggaaagattgctgatggttcttcaggtgcagtcgcctgtgacagctataacaggacagccgaagacatagctttattaaaagacctgggtgttaccgcatatagattttccattagttggtccagaataatcccattgggtggcaggaacgatcctataaatcaagctggtatagaccattatgtgaaatttgtcgatgatctaacagacgctgggatcactcctttcgttacgttgtttcactgggatcttcctgacggattagataaaagatacggcggtctattgaacagggaagaatttccactagactttgaacactacgcaagaactatgttcaaggcgctaccaaaagtgaagcactggatcactttcaatgagccttggtgctcggccattttgggttacaatacgggtttcttcgctccaggccatacttctgatcgtagcaagtctgctgttggtgatagcgcacgtgagccatggatcgctgggcacaatatgttggtagcccacggaagagcggtaaaaacgtacagagaagattttaagcccacaaacggtggtgaaattggtattactttaaacggtgatgccacatacccttgggaccctgaagaccccgaagacgttgccgcttgcgacagaaagatagaatttgcaatctcctggttcgccgacccgatttatttcggcaaataccctgattcaatgttagctcaattaggtgatagacttcctacctttaccgatgaggagagagcattggttcagggtagcaatgatttttacggtatgaatcattacaccgcgaattatattaaacataagactgggacaccacccgaggatgatttcttgggcaacctggaaacattgttcgactccaaaaacggtgagtgtatagggcctgaaacgcaatctttttggctgaggcccaatccccagggttttagggatttgctaaattggttgtctaagagatacggatatccgaaaatttatgtcacagagaatggaacatctttaaagggggaaaatgatatggaaagagatcaaattttggaggatgatttcagagtcgcctattttgacggctatgtgagggctatggcagaagctagtgagaaagatggcgttaatgttcgtggatatctagcatggtcactattagataatttcgaatgggctgagggctacgagactagatttggcgttacctatgttgattatgagaacgggcaaaagagataccctaagaaatctgctaaatcgttgaagcctctgtttgatagcttgataaaaactgattaa

SEQ ID NO: 76 - DNA, 1503 bp, Lomentospora prolificans
atgagaaaaggtattgttttggctgttgttttggttgttttgagagttcaaacttgtattgctcaaattaatagagcttcttttccaaaaggttttgtttttggtactgcttcttctgcttatcaatatgaaggtgctgttaaagaagatggtagaggtcaaactgtttgggatgaatttgctcattcttttggtaaagttttggatttttctaatgctgatattgctgttaatcaatatcatttgtttgatgaagatattaaattgatgaaagatatgggtatggatgcttatagattttctattgcttggtctagaatttttccaaatggtactggtgaaattaatcaagctggtgttgatcattataataatttgattaatgctttgttggctaatggtattgaaccatatgttactttgtatcattgggatttgccacaagctttggaagatagatataatggttggttgcatccacaaattattaaagattttgctttgtatgttgaaacttgttttgaaaaatttggtgatagagttaaacattggattacttttaatgaaccacatacttttactattcaaggttatgatgttggtttgcaagctccaggtagatgttctattttgttgcatattttttgtagaggtggtaattctgctattgaaccatatattattgctcataatgttttgttgtctcatgctactgttgttgatatttatagaagaaaatataaaccaaaacaacatggttctgttggtgtttcttttgatgttatttggtttgaaccagctactaattctactgttgatattgaagctgctcaaagagctcaagattttcaattgggttggtttattgaaccattgatttttggtgaatatccatcttctatgattactagagttggttctagattgccaagatttactaaagctgaatctgctttgttgaaaggttctttggattttattggtattaatcattatactactttttatgctaaaccaaatacttctaatattattggtgttttgttgaatgattctattgctgattctggtgctattactttgccatttagagatggtactccaattggtgatagagctaattctatttggttgtatattgttccacatggtattagatctttgatgaattatattaaacaaaaatatggtaatccaccagttattattactgaaaatggtatggatgatgctaattctccattgatttctttgaaagatgctttgaaagatgaaaaaagaattaaatatcataatgattatttggaatctttgttggcttctattaaagatgatggttgtaatgttaaaggttattttgtttggtctttgttggataattgggaatgggctgctggtttttcttctagatttggtttgtattttgttgattatggtgataaattgaaaagatatccaaaagattctgttaaatggtttaaaaattttttgacttctgcttaa

SEQ ID NO: 77 - DNA, 1482 bp, Heliocybe sulcata
atggctcaaaaattgccatctgattttttgtggggtatggctactgcttcttatcaaattgaaggttctccagatgctgatggtagaggtccatctatttgggatactttttctcatttgccaggtaaaactttggatggtttgactggtgatattgctactgattcttatagattgagagatcaagatattgctttgttgaaacaatatggtgttaaatcttatagattttctatttcttggtctagagttattccattgggtggtagaaatgatccaattaatgaaaaaggtattaaatggtattctgatttgattgatgaattgttggaagctggtattgttccatttgttactttgtatcattgggatttgccacaagctttgcatgatagatatggtggttggttgaataaagatgaaattgttgctgattttgttaattatgctagattgtgttttgaaagatttggtgatagagttaaatattggttgacttttaatgaaccatggtgtatttctattttgggttatggtagaggtgtttttgctccaggtagatcttctgatagaactagatctccagaaggtgattctagaactgaaccatggattgttggtcattctgttattgttgctcatgcttctgctgttaaattgtatagagatgaatttaaatctagacaacatggtgttattggtattactttgaatggtgatatggctttgccatgggatgattctgaagaatgtagacaagctgctcaacatgctttggatgttgctattggttggtttgctgatccagtttatttgggtcattatccaccatttatgagacaatttttgggtgatagattgccaacttttactccagaagaagaaaaattggttaaaggttcttctgatttttatggtatgaatacttatactactaatttgattagaccaggtggtgatgatgaatttcaaggtaatgttcaatatacttttactagaccagatggttctcaattgggtactcaagctcattgtgcttggttgcaaacttatccagaaggttttagagctttgttgaattatttgtggaatagatatcatatgccaatttatgttactgaaaatggttttgctgttaaaaatgaaaataatatgccattggaacaagctttgaaagatactgatagaattgaatattttaaaggtaattgtgaagctttggttaaagctgttcatgaagatggtgttgatttgagaggttattttccatggtcttttttggataattttgaatgggctgatggttatcaaactagatttggtgttacttatgttgattatgctactcaaaaaagatatccaaaagaatctgcttggtttttggttaattggtttaaagaaaatgttaattctccaaaatcttctggtgaaccaagaacttctagaattccaaatggtgctgttccaaatggtcatatttaa

SEQ ID NO: 78 - DNA, 1410 bp, Moniliophthora roreri MCA 2997
atgaaattgccaaaagattttttgtttggttatgctactgcttcttatcaaattgaaggttcttctgatgttgatggtagaggtccatctatttgggatactttttctcatactccaggtaaaattgttgatggtactaatggtgatgttgctactgattcttatcaaagatggaaagatgatgttaaaattgttaaagattatggtgctaatgcttatagattttctatttcttggtctagaattattccattgggtggtaaagatgatccagttaatccagaaggtattagattttatagaactttgattgaagaattgttgaataatggtattactccatgtgttactttgtatcattgggatttgccacaagctttgcatgatagatatggtggttggttggatagaagagttattgaagattttgttagatattgtgaaatttgttttgaagcttttggtaattctgttaaacattggattacttttaatgaaccatggtgtatttcttgtttgggttatggttatggtgtttttgctccaggtagatcttctaatagaaatagatctgaagctggtgattctactagagaaccatggattgttgctcataatttgttgttggctcatgcttctgctgttgcttcttatagacaaaaattttggccatctcaagctggttctattggtattactttggattgtgtttggtatatgccatatgatgaatctaatgctgaagatgttgatgctgctcaaagagctttggatactagattgggttggtttgctgatccaatttataaaggtcattatccaacttctttgaaagctatgttgggtaatagattgccagaatttactactgaagaacaagctttgattaaaggttcttctgatttttttggtttgaatacttatacttctaatttggttcaaccaggtggttctgatgaatttaatggtaaagttaaaactactcatactagagctgatggttctcaattgggtaaacaagctcatgttccatggttgcaagcttatccaccaggttttagagctttgttgaattatttgtggaaaacttatggtaaaccaatttatgttactgaaaatggttttgctattaaagatgaaaatagattgccaccagaagatgctattcatgatcaagatagagttgattattatagaggttatactaatgctttggctcatgctgctaatgaagatggtgttgatgttaaagcttattttgcttggtctttgttggataattttgaatgggctgaaggttatcaagttagatttggtgttacttttgttgattttgaaactcaacaaagatatccaaaagattcttctaaatttttggctgaatggtatagatcttctttggctaaataa

SEQ ID NO: 79 - DNA, 1623 bp, Rauvolfia serpentina
atggctactcaatcttctgctgttattgattctaatgatgctactagaatttctagatctgattttccagctgattttattatgggtactggttcttctgcttatcaaattgaaggtggtgctagagatggtggtagaggtccatctatttgggatacttttactcatagaagaccagatatgattagaggtggtactaatggtgatgttgctgttgattcttatcatttgtataaagaagatgttaatattttgaaaaatttgggtttggatgcttatagattttctatttcttggtctagagttttgccaggtggtagattgtctggtggtgttaataaagaaggtattaattattataataatttgattgatggtttgttggctaatggtattaaaccatttgttactttgtttcattgggatgttccacaagctttggaagatgaatatggtggttttttgtctccaagaattgttgatgatttttgtgaatatgctgaattgtgtttttgggaatttggtgatagagttaaacattggatgactttgaatgaaccatggactttttctgttcatggttatgctactggtttgtatgctccaggtagaggtagaacttctccagaacatgttaatcatccaactgttcaacatagatgttctactgttgctccacaatgtatttgttctactggtaatccaggtactgaaccatattgggttactcatcatttgttgttggctcatgctgctgctgttgaattgtataaaaataaatttcaaagaggtcaagaaggtcaaattggtatttctcatgctactcaatggatggaaccatgggatgaaaattctgcttctgatgttgaagctgctgctagagctttggattttatgttgggttggtttatggaaccaattacttctggtgattatccaaaatctatgaaaaaatttgttggttctagattgccaaaattttctccagaacaatctaaaatgttgaaaggttcttatgattttgttggtttgaattattatactgcttcttatgttactaatgcttctactaattcttctggttctaataatttttcttataatactgatattcatgttacttatgaaactgatagaaatggtgttccaattggtccacaatctggttctgattggttgttgatttatccagaaggtattagaaaaattttggtttatactaaaaaaacttataatgttccattgatttatgttactgaaaatggtgttgatgatgttaaaaatactaatttgactttgtctgaagctagaaaagattctatgagattgaaatatttgcaagatcatatttttaatgttagacaagctatgaatgatggtgttaatgttaaaggttattttgcttggtctttgttggataattttgaatggggtgaaggttatggtgttagatttggtattattcatattgattataatgataattttgctagatatccaaaagattctgctgtttggttgatgaattcttttcataaaaatatttctaaattgccagctgttaaaagatctattagagaagatgatgaagaacaagtttcttctaaaagattgagaaaataa

SEQ ID NO: 80 - DNA, 1431 bp, Pyricularia grisea
atgtctttgccaaaagattttttgtggggttttgctactgcttcttatcaaattgaaggtgctattgataaagatggtagaggtccatctatttgggatacttttactgctattccaggtaaagttgctgatggttcttctggtgttactgcttgtgattcttataatagaactcaagaagatattgatttgttgaaatctgttggtgctcaatcttatagattttctatttcttggtctagaattattccaattggtggtagaaatgatccaattaatcaaaaaggtattgatcattatgttaaatttgttgatgatttgttggaagctggtattactccattgattactttgtttcattgggatttgccagatggtttggataaaagatatggtggtttgttgaatagagaagaatttccattggattttgaacattatgctagagttatgtttaaagctattccaaaatgtaaacattggattacttttaatgaaccatggtgttcttctattttggcttattctgttggtcaatttgctccaggtagatgttctgatagatctaaatctccagttggtgattcttctagagaaccatggattgttggtcataatttgttggttgctcatggtagagctgttaaagtttatagagaagaatttaaagctcaagataaaggtgaaattggtattactttgaatggtgatgctacttttccatgggatccagaagatccaagagatgttgatgctgctaatagaaaaattgaatttgctatttcttggtttgctgatccaatttattttggtgaatatccagtttctatgagaaaacaattgggtgatagattgccaacttttactgaagaagaaaaagctttggttaaaggttctaatgatttttatggtatgaattgttatactgctaattatattagacataaagaaggtgaaccagctgaagatgattatttgggtaatttggaacaattgttttataataaagctggtgaatgtattggtccagaaactcaatctccatggttgagaccaaatgctcaaggttttagagaattgttggtttggttgtctaaaagatataattatccaaaaattttggttactgaaaatggtacttctgttaaaggtgaaaatgatatgccattggaaaaaattttggaagatgattttagagttcaatattatgatgattatgttaaagctttggctaaagcttattctgaagatggtgttaatgttagaggttattctgcttggtctttgatggataattttgaatgggctgaaggttatgaaactagatttggtgttacttttgttgattatgaaaatggtcaaaaaagatatccaaaaaaatctgctaaagctatgaaaccattgtttgattctttgattgaaaaagattaa

SEQ ID NO: 81 - DNA, 1605 bp, Ophiorrhiza pumila
atggagttcttaaaccctgcattcacacgtgtcccttcgggattcttaaggcgtaaggacttcggctcggacttcatattcggatcagcaaccagcgccttccaggtcgagggtggaatgagggaagacggacgtggaccgtcaatatgggactcgttcgcggagaagaggaacttattcgccccttactcagaggacgcgatcaaccaccacaagaactacgaagaggacgtcaagctaatgaaggagatcggcttcgacgcatacaggttctccatatcatggaccaggatactgcctaccggaaagaaggagtcacgtaaccagaagggcatcgacttctacaagaagttacttaagaacttaaagataaaggggatcgagccctacgtcacgctattacacttcgacccacctcagaacttagaggacaagtactacggcttccttaaccgtcagatcgcggacgacttctgcgactacgcagacatatgcttcaaggagttcgggaacgacgtcaagcactggataaccatcaacgagccgtggagcttcgcatacggtgggtacttcacaggaaacttagcgcctggctaccacgcgcagacagacaagatagcccctcaccagtccacgaagatcccgaacgacgacgacgacgacgcacaccacaagtcatccatattcccgccttcgcgtttcagccttccaccttcaagctcctcagcgagcgagacacctgccatcatcccggccaagaagttaccctaccctgacgtcaacaagtacccctaccttgtcgcgcaccaccagatactggcacacgcaaaggccgtgaagttataccgtcagaactaccagaggacacagaagggcaagataggaatagtcctggtatcgcagtggtacatctcgctggacgacgaccccgacaacaaagaggccacccagagggccaacgacttcatgctgggctggttccttgaccccatattctccggcgactaccctgcgtcaatgaggaagtacgtgacaaagggatacttacccgagttctcctcggcggacaaggagatgataaagggctcattcgacttcttaggcttaaactactacacagccaggtacgtaacatacgaggagacaggcggtggaaactacgtcctggaccagagggcaaggttccacgtcaagaggaagggcaagttaataggcgacgagaagggcgcttccgggtggatatacggatacccccgtggaatgctagacctacttgtatacatgaaggagaagtacaacaagcctacgatatacatcacagagacaggaatcgacgacccggacgacgacagttcaacacactggaagtcattctacgaccaggaccgtataatgttctaccacgaccacctatcatacataaagcaggccatgaggaagggcgtgaacgtcaagggcttcttcgcctggtcactgatggacaacttcgagtgggacgtcggcttcaagtcgaggttcgggataacatacatcgacttcgaggacggctccaagaggtgccctaagctttcagcatcctggttcaagtacttcttagagaactga

SEQ ID NO: 82 - DNA, 1413 bp, Hydnomerulius pinastri MD-312
atgactgaagctaaattgccaaaagattttacttggggttttgctactgcttcttatcaaattgaaggtgcttataatgaaggtggtagagctgattctatttgggatacttttactagattgccaggtaaaattgctgatggttcttctggtgaagttgctactgattcttatcatagatggaaagaagatgttgctttgttgaaatcttatggtgttaattcttatagattttctttgtcttggtctagaattattccattgggtggtagagaagataaagttaatgctgaaggtgttgctttttatagaaattttgctcaagaattggttaaaaatggtattactccatatatgactttgtatcattgggatttgccacaagctttgcatgatagatatggtggttggttgaataaagaagaaattgttaaagattatgttaattatgctaaagtttgttatgaatcttttggtgatattgttaaacattggattactcataatgaaccatggtgtgtttctgttttgggttatggtaaaggtgtttttgctccaggtcatacttctgatagagctaaatttcatgttggtgattcttctactgaaccatatattgttgctcattctatgttgttggctcatggttatgctgttaaattgtatagagaacaatttcaaccacaacaaaaaggtactattggtattactttggattcttcttggtttgaaccattgactaatactcaagaaaatgctgatgttgctcaaagagcttttgatgttagattgggttggtttgctcatccaatttatttgggttattatccagaagctttgaaaaaacaatgtggttctagattgccagaatttactgctgaagaaattgctgttgttaaaggttcttctgatttttttggtttgaatcattatactactcatttggtttctgaaggtggtgatgatgaatttaatggttatgctaaacaaactcataaaagagttgatggtactgatattggtactcaagctgatgttaattggttgcaaacttatggtccaggttttagaaaattgttgggttatatttataaaaaatatggtaaaccaattattattactgaatctggttttgctgttaaaggtgaaaattctaaaactattgaagaagctattaatgatactgatagagaagaatattatagagattatactaaagctatgttggaagctgttactgaagatggtgttgatgttaaaggttattttgcttggtctttgttggataattttgaatgggctgaaggttatagaattagatttggtgttacttatgttgattataaaactcaaaaaagatatccaaaacattcttctaaatttttgaaagaatggtttgctgctcatatttaa

SEQ ID NO: 83 - DNA, 1671 bp, Helianthus annuus
atggcgacgttcgacttaaccgaccagatagcaccgttccctgacgagataagctccgccgacttcgatagtgacttcgtgtggggcgcggccacatcagcgtaccagatagaaggtgctgcgtgcgagggtgggaagggccctagcatctgggacgtcttctgcttaaccgaccctgggcgtatagtcggtggcgacaacgggaacatcgcggtcaacagttactacaagacaaaagaggacgtacagacaatgaagaagatggggctacaggcgtaccgtttcagtctaagctggagtaggatactaccgggtgggaagcttaagttaggcatcaaccaagagggcgtagactactacaacaaccttataaacgagcttctagcaaacgacatcgagccttacgtcaccttatggcactgggacacacccaacgtcctagaggccgagtacggcggattcctttgcgagaagatagtctacgacttcgtgaactacgtcgagttctgcttctgggagttcggcgaccgtgtcaagcactggacaaccctgaacgaaccccacagctatgtagagaaggggtacacgacgggcaagtttgcacctggccgtggtggcgaggggatgcccggcaaccccgggaccgagccttacatcgtagggcactacctattattaagtcacgcgaaggccgtggacttataccgtaggcgtttccaggcatcacagggcggcacaataggaatcacgttaaacaccaagttctacgagccccttaactcggagctacaggacgacatcgacgcagcgttaagggccatagacttcatgctgggatggttcatggagcccctattcagtgggaagtaccctgacacaatgatcgagaacgtgacagacgacaggctgcctacattcacaaaggagcagtccgagttagtgaagggcagttacgacttcttagggctaaactactacgcatcccagtacgccaccaccgcccctgagaccaacgtggtgagtctgttaaccgacagcaaggtattagagcagcctgacaacatgaacggaatacctatcggaataaaggcaggactggactggctttactcatatccccctggcttctacaagctgcttgtatacataaaggacacatacggcgaccccttaatctacataaccgagaacgggtgggtggacaagaccgacaacacaaagacagtggaagaggcacgtgtagacctggagaggatggactaccacaacaagcaccttcagaacttaaggtacgccatcagtgcaggagtacgtgtcaaggggtacttcgtctggagtcttatggacaacttcgagtgggacgagggctactccgcgcgtttcggacttatctacatagacttcaagggcggaaagtacacacgttaccccaagaactccgcaatatggtacaagcacttcttaggctactccaacaagcagaagacggagaagaagaagaaccttgcacgtgagcgtacctgcaagtcatcggagaagacaacaaagttcgagcttgagctagagaacaactgctactgccttgacctactatccttcttattaccgaggatcaacatgaaggtgaactacaagttcggcggggtcaagttaaaggacgagcagcgttga

SEQ ID NO: 84 - DNA, 1518 bp, Actinidia chinensis var. chinensis
atggctattaatagagctttgttgattttgttttgttttttggctatttctaatactgaagctacttctaaaaaatatccaccattgggtagatcttcttttccaaaagattttgtttttggtgctggttctgctgcttatcaatttgaaggtggtgcttttattgatggtaaaggtgattctatttgggatacttttactcatcaacatccagaaaaaattgctgatagatctaatggtactattgctgatgatatgtatcatagatataaaggtgatgttgctttgatgaaaactactggtttggatggttttagattttctatttcttggtctagagttttgccaaaaggtagagtttctggtggtgttaatgctttgggtgttaaatattataataatttgattaatgaaattttggctaatggtatggttccatatgttactatttttcattgggatttgccacaagctttggaagatgaatatactggttttagaaataaaaaaattgttgatgattttagagattatgctgaatttttgtttaaaacttttggtgatagagttaaacattggtttactttgaatgaaccatatacttattcttattttggttatggtactggtactatggctccaggtagatgttctaattatgttggtacttgtactgaaggtgattcttctactgaaccatatattgttactcatcatttgattttggctcatggtgctgctgttaaattgtatagagaaaaatataaaccatatcaaagaggtcaaattggtgttactttggttactgcttggtttgttccaactactgctactactacttctgaaagagctgctagaagagctttggattttatgtttggttggtttttgcatccaatgacttatggtgattatccaatgactttgagagctttggctggtaatagagttccaaaatttactgctgaagaaactgctatgttgcaaaaatcttatgattttttgggtgttaattattatactgctttttttgcttctaatgttatgttttctaattctattaatatttctatgactactgataatcatgctaatttgacttctgttaaagatgatggtgttgctattggtcaatctactgctttgaattggttgtatgtttatccaaaaggtatggaagatttgatgttgtatttgaaagataattatggtaatccaccaatttatattactgaaaatggtattgctgaagctaataatgataaattgccagttaaagaagctttgaaagataatgatagaattgaatatttgtattctcatttgttgtatttgtctaaagctattaaagctggtgttaatgttaaaggttattttatgtgggcttttatggatgattttgaatgggatgctggttttactgttagatttggtatgtattatattgattataaagatggtttgaaaagatatccaaaatattctgcttattggtataaaaaatttttgcaaacttaa

SEQ ID NO: 85 - DNA, 1695 bp, Handroanthus impetiginosus
atggaaaacggttctggtgctgttgtagccgtaggcaatccacagagtgccggttccccaaatgccgttcccccagatcaagataattcgaacataaatagggatgattttcccaatgattttgtattcggatccggaacctctgcttttcaagttgaaggcgctgcagctctggacgggaaggcaccgtccgtttgggatgacttcacattaagaactccgggtagaatagctgatgggtcaaacggaattgtcgcagctgacatgtaccataaatataaagaagacattcgtaatatgaagaaaatgggattcgatgtttataggttcagtatcagttggcctagaattttaccgggtggtagatgttcagctggcatcaatagactaggcattgattattataatgacctgattaacaccataattgcgcacggtatgaaaccttttgtaactctattccattgggatttaccagatattttggaaaaagaatacaatggatttctatctcgtaagattctagatgatttcttggagtacgctgagttatgtttttgggagttcggagatagggttaagttctggacaaccatcaatgaaccttggtcagtagccgttaatggatacgttagaggcaccttcccaccatcgaaagcatcttgtccaccagatagagtcttaaagaaaattccaccacatagatcagtccaacattcatccgctaccgtacctacgaccaggcaatactcggatatcaaatacgacaagagcgatccggctaaggatccttacacggttgggagaaatttactattgattcatgctaaggttgtatgtctgtatagaacaaaatttcaggggcatcaaagaggacaaattggtattgtgcttaactctaattggtttgttccaaaagacccagattcggaagctgatcagaaggctgccaagagaggagtggattttatgctaggctggttcctacatcctgtactttatgggtcttacccgaagaatatggtagactttgtgccagccgagaatcttgctcccttttctgaacgtgaatccgacttgcttaaaggatctgctgattacattggacttaatttttatacagccttgtatgcagaaaatgatccgaaccctgagggtgtcggttacgatgctgatcaaagggtcgttttctctttcgataaagatggcgtccccataggtcctcccacaggaagttcatggctgcatgtttgtccttgggccatctacgatcatttagtctacttgaagaaaacatatggtgatgcacctcccatttacattactgaaaatggtatgtctgataaaaacgatccaaaaaaaacagccaaacaagcctgctgtgactctatgagagttaagtatcatcaagatcatcttgctaatatattgaaagccatgaacgatgtacaagttgacgtgcgtggttacatcatctggtcgtggtgcgataattttgaatgggcagaaggttatacggttagatttggaataacttgcattgattacttgaatcaccaaaccagatatgcaaaaaattccgctttatggttctgtaagttccttaagtcaaaaaagagtcagattcaaagttccaataaaagacaaatcgagaacaactccgaaaatgttttggcgaaaaggtataaggtgtaa

SEQ ID NO: 86 - DNA, 1632 bp, Carapichea ipecacuanha
atgtcgtcagtcctacctacacccgtcttacctacacctggaaggaacatcaaccgtggccacttcccggacgacttcatcttcggggcaggaacatcaagctaccagatagaaggggccgcaagagagggcggaaggggaccttcaatatgggacaccttcacccacacgcaccctgagttaatacaggacggctcgaacggcgacacggccataaactcctacaacctatacaaagaggacatcaagatagtaaagcttatgggcctagacgcatacaggttcagtataagttggcctaggatcctgcctggcggctcaataaacgccggaatcaaccaagagggcataaagtactacaacaacctgatagacgagctattagccaacgacatcgtgccttacgtgacacttttccactgggacgtgcctcaggcacttcaggaccagtacgacggattcctaagcgacaagatagtagacgacttccgtgacttcgcagagctgtgcttctgggagttcggagaccgtgtcaagaactggataaccataaacgagcccgagtcgtacagtaacttcttcggagtggcctacgacacacccccgaaggcacacgccctgaaggcatcaaggttattagtgcctacgacagtagcacgtccttccaagcctgtgagggtcttcgcgtccacggcagaccccggcacaacgaccgcggaccaggtatacaaggtcggacacaacttactactagcacacgccgcggcaatacaggtgtaccgtgacaagttccagaacacgcaagagggaacgttcggcatggcacttgtcacccagtggatgaagcctctaaacgagaacaacccggcagacgtcgaggcagcatcccgtgcattcgacttcaagttcggctggttcatgcagcctttaatcacaggcgagtaccctaagtccatgcgtcagttattagggccgcgtttaagggagttcaccccggaccagaagaagcttttaatcggctcgtacgactacgtaggagtaaactactacacagccacatacgtcagtagtgcacagccgccccacgacaagaagaaggccgtgttccacaccgacggcaacttctacaccacagacagtaaggacggggtcctaatcggacctcttgccggccctgcatggttaaacatagtccctgaggggatataccacgtgcttcaggacataaaggagaactacgaggaccccgtcatatacataaccgagaacggagtgtacgaggtaaacgacacagccaagaccttaagtgaggcacgtgtggacacaacacgtttacactacttacaggaccacttatcaaaggtattagaggcgaggcaccagggcgtgagggtacagggatacctagtgtggtcattaatggacaactgggagctaagggccggctacacttcccgtttcggcctaatacacatagactactacaacaacttcgcaaggtacccgaaggactcagccatatggttcaggaacgcgttccacaagaggctaaggatacacgtgaacaaggcccgtccccaggaagacgacggagccttcgacaccccgaggaagaggctaaggaagtactaa

SEQ ID NO: 87 - DNA, 1668 bp, Lactuca sativa
atggagaccacgacacagaacacgggcgccaagttctcactattccagaaccttgtccactcaaacgacttcaagcccgacttcgtatggggcgcagccacaagtgcctaccagatagagggagccgccagcaagggtggaaggggagagtcaatatgggacgtattctgccacaacaaccccgacgccatcgtgaacggggacaacggcaacaacggaacgaacgcatacttcaagtacaaagaggacgtccagatgatgaagaagatgggactgaacgcatacaggttctccatctcgtggacgcgtatattcccgggagggaggccctcaaacggcataaacaaggaaggcatagactactacaacaacctgataaacgagctaatcctatgcggcataacgccttacgtaaccctattccactgggacacacctgagaccttagaggaagagtacatgggcttcctatccgagaagataatatacgacttcacctcatacgcaggcttctgcttctgggagttcggggaccgtgtaaagaactggataacaataaacgagcctcacagctacgcatcgtgcggatacgcagacggcacattcccacctggacgtggcaaggacggagtaggcgaccccggaacagagccttacatcgtcgcaaagaacctgttactgagccacgcatccgtcgtaaacttatacaggcagaagttccagaagaagcagggtgggaagatcggaataacccttaacgcagtgttctgcgagccgttaaaccctgagaagcaggaagacaaggacgcagcattacgtgccatagacttcatgttcggatggttcatggagcctctgttctccgggaagtaccccgacaacatgataaagtacgtaacaggagaccgtttacctgagttcacagccgaggaagccaagtccataaagggatcatacgacttcttaggcctgaactactacacatcatactacgccacatcagcaaagccttcacaggtgcctagctacgtgacggactccaacgtccaccagcaggcggaaggcttagacggcaagcccatagggccgcagggcggcagcgattggttatacagttacccgctaggcttctacaagatcttacagcacataaagcacacctacggggacccgcttatcttcatcaccgagaacggctggccggacaagaacaacgacaccatcggcatcggggcagcatgcgtggacacgcagaggatagactaccacaacgcgcacctgcagaagcttcgtgacgccgtaagggacggagtcagggtggaagggtacttcgtgtggagtctaatggacaacttcgagtggatagccggatactcaatacgtttcggactgctatacgtcgactacaacgacggaaagtacaccaggtaccccaagaactcagccatatggtacatgaacttcttaaagtcccctaagaagttaggggagcagaagaagatccctaagtgcgtccccaacaagcctatagcgaagacacagagtaccgagacatcgaccaagacaagtcgtgtgcttgccgaggtagtgttaatcatgatcttatcgatcctgtgcatcgtcatgttcatcttcgactacaagatgaagataggatgcatatactga

SEQ ID NO: 88 - DNA, 1611 bp, Coffea arabica
atggccgccaagagcaacgtcacaaacgacctaagtagggcggatttcggtgaggacttcatcttcggaagcgcttccgcggcctaccagatggaaggagcagccgaagagggcgggcgtggccctagtatatgggacaagttcacggagcagaggccggacaaggtagtagacggatcaaacgggaacgtagcaatcgaccagtaccacaggtacaaggaagacgtgcagatgatgaagaagatcgggttagacgcatacaggttctcaatctcctggagtagggtgcttcctggtggaaggttaaacgcaggcgtgaacaaagagggaatacagtactacaacaacttaatcgacgagcttctggcaaacggaatcaagcctttcgtgacattattccactgggacgtaccccagacactggaagacgagtacggtggattcttatgcaggagaatcgtagacgacttccgtgagttcgcggagttatgcttctgggagttcggagaccgtgtcaagcactggatcacccttaacgagccttggaccttcgcctacaacggatacacaaccggtggacacgcacccggaagagggatatcaaccgcagagcacataaaggacgggaacacaggacacaggtgcaaccacttattctcagggatccctgtagacggaaaccctggaacggagccgtacttagtagcacaccacttacttcttgcacacgcagaggcagtcaaggtgtacagggagacattcaagggccaagagggaaagatcggaataacactagtgtcacagtggtgggagcctttaaacgacacaccccaggacaaagaggccgtagagcgtgcggccgacttcatgttcggatggttcatgtcccctatcacatacggggactaccctaagcgtatgagggacatcgtcaagtcacgtctacccaagttctccaaagaggagagccagaacctaaaggggagtttcgacttcttaggacttaactactacacctcgatctacgccagtgacgcgtcaggcacgaagagcgagctactgagttacgtaaacgaccagcaggtaaagacacagacagtaggccccgacggaaagaccgacatagggcccagggccggatcagcctggctatacatctaccccctaggaatctacaagctattacagtacgtgaagacccactacaactcacctcttatatacatcacggagaacggagtagacgaggtaaacgaccctggattaacagtatccgaggcccgtatcgacaagacacgtataaagtaccaccacgaccaccttgcgtacgtgaagcaggcaatggacgtcgacaaggtgaacgtaaagggctacttcatctggtcactacttgacaacttcgagtggtcagagggctacacggcaaggttcgggatcatacacgtcaacttcaaggacaggaacgcgaggtaccctaagaagtccgcattatggttcatgaacttcttagccaagtccaacctaagtccgacaaagacaacgaagagggccttagacaacggtggacttgcagacctagagaaccctaagaagaagatattaaagacatga

SEQ ID NO: 89 - protein, 115 aa, Artificial sequence
  Domain 1 of RseSGD from Rauvolfia serpentina
  DOMAIN (1)..(115): Domain 1 of RseSGD from Rauvolfia serpentina
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYR

SEQ ID NO: 90 - protein, 151 aa, Artificial sequence
  Domain 2 of RseSGD from Rauvolfia serpentina
  DOMAIN (1)..(151): Domain 2 of RseSGD from Rauvolfia serpentina
FSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEG

SEQ ID NO: 91 - protein, 189 aa, Artificial sequence
  Domain 3 of RseSGD from Rauvolfia serpentina
  DOMAIN (1)..(189): Domain 3 of RseSGD from Rauvolfia serpentina
IGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDG

SEQ ID NO: 92 - protein, 76 aa, Artificial sequence
  Domain 4 of RseSGD from Rauvolfia serpentina
  DOMAIN (1)..(76): Domain 4 of RseSGD from Rauvolfia serpentina
VNVKGYFVWSFFDNFEWNLGYICRYGIIHVDYKSFERYPKESAIWYKNFIAGKSTTSPAKRRREEAQVELVKRQKT

SEQ ID NO: 93 - protein, 540 aa, Artificial sequence
  CCRR
  PEPTIDE (1)..(540): CCRR
MGSKDDQSLVVAISPAAEPNGNHSVPIPFAYPSIPIQPRKHNKPIVHRRDFPSDFILGAGGSAYQCEGAYNEGNRGPSIWDTFTNRYPAKIADGSNGNQAINSYNLYKEDIKIMKQTGLESYRFSISWSRVLPGGNLSGGVNKDGVKFYHDFIDELLANGIKPFATLFHWDLPQALEDEYGGFLSDRIVEDFTEYAEFCFWEFGDKVKFWTTFNEPHTYVASGYATGEFAPGRGGADGKGNPGKEPYIATHNLLLSHKAAVEVYRKNFQKCQGGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 94 - protein, 540 aa, Artificial sequence
  CRRR
  PEPTIDE (1)..(540): CRRR
MGSKDDQSLVVAISPAAEPNGNHSVPIPFAYPSIPIQPRKHNKPIVHRRDFPSDFILGAGGSAYQCEGAYNEGNRGPSIWDTFTNRYPAKIADGSNGNQAINSYNLYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 95 - protein, 532 aa, Artificial sequence
  CRRC
  PEPTIDE (1)..(532): RCRR
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGNLSGGVNKDGVKFYHDFIDELLANGIKPFATLFHWDLPQALEDEYGGFLSDRIVEDFTEYAEFCFWEFGDKVKFWTTFNEPHTYVASGYATGEFAPGRGGADGKGNPGKEPYIATHNLLLSHKAAVEVYRKNFQKCQGGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 96 - protein, 534 aa, Artificial sequence
  RRRC
  PEPTIDE (1)..(534): RRRC
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGFFVWSFFDNFEWNLGYICRYGIIHVDYKTFQRYPKDSAIWYKNFISEGFVTNTAKKRFREEDKLVELVKKQKY

SEQ ID NO: 97 - protein, 534 aa, Artificial sequence
  RCRC
  PEPTIDE (1)..(534): RCRC
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGNLSGGVNKDGVKFYHDFIDELLANGIKPFATLFHWDLPQALEDEYGGFLSDRIVEDFTEYAEFCFWEFGDKVKFWTTFNEPHTYVASGYATGEFAPGRGGADGKGNPGKEPYIATHNLLLSHKAAVEVYRKNFQKCQGGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGFFVWSFFDNFEWNLGYICRYGIIHVDYKTFQRYPKDSAIWYKNFISEGFVTNTAKKRFREEDKLVELVKKQKY

SEQ ID NO: 98 - protein, 542 aa, Artificial sequence
  CCRC
  PEPTIDE (1)..(542): CCRC
MGSKDDQSLVVAISPAAEPNGNHSVPIPFAYPSIPIQPRKHNKPIVHRRDFPSDFILGAGGSAYQCEGAYNEGNRGPSIWDTFTNRYPAKIADGSNGNQAINSYNLYKEDIKIMKQTGLESYRFSISWSRVLPGGNLSGGVNKDGVKFYHDFIDELLANGIKPFATLFHWDLPQALEDEYGGFLSDRIVEDFTEYAEFCFWEFGDKVKFWTTFNEPHTYVASGYATGEFAPGRGGADGKGNPGKEPYIATHNLLLSHKAAVEVYRKNFQKCQGGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGFFVWSFFDNFEWNLGYICRYGIIHVDYKTFQRYPKDSAIWYKNFISEGFVTNTAKKRFREEDKLVELVKKQKY

SEQ ID NO: 99 - protein, 531 aa, Artificial sequence
  VVRR
  PEPTIDE (1)..(531): VVRR
MESNQGEPLVVAIVPKPNASTEQKNSHLIPATRSKIVVHRRDFPQDFVFGAGGSAYQCEGAYNEGNRGPSIWDTFTQRTPAKISDGSNGNQAINCYHMYKEDIKIMKQAGLEAYRFSISWSRVLPGGRLAAGVNKDGVKFYHDFIDELLANGIKPFATLFHWDLPQALEDEYGGFLSHRIVDDFCEYAEFCFWEFGDKIKYWTTFNEPHTFTANGYALGEFAPGRGKNGKGDPATEPYLVTHNILLAHKAAVEAYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 100 - DNA, 1623 bp, Artificial sequence
  CCRR
  misc_feature (1)..(1623): CCRR
atgggcagcaaagatgatcagagtttagtagttgcgatatctccagctgctgaaccaaacggaaatcatagtgtgcccattccatttgcttaccctagcatcccaatccagccaagaaaacataataaaccaatagttcatagaagagattttccatcagacttcatcctaggagctggaggcagtgcgtatcagtgtgaaggtgcatataacgaaggtaatagagggccgtcaatttgggatactttcacaaaccgttaccctgcgaagatagcagatggcagtaatggcaatcaagccatcaactcttacaatttgtacaaggaagacattaaaataatgaaacaaaccgggcttgaaagttatagattttccatttcttggtctagagttttaccaggaggtaaccttagcggaggcgttaataaggatggagtgaagttttatcatgacttcatcgacgaactgctggctaatggtatcaaaccatttgctacgctgtttcactgggacctaccacaggctttggaagatgagtacggtggtttcttatctgacagaattgtcgaagattttactgaatatgctgaattttgtttctgggaatttggagacaaagtaaaattctggaccacgtttaatgaaccccatacttatgtagcgagcggttacgcaactggagaatttgctcctggaagagggggcgccgatggaaaaggcaacccaggtaaggaaccatacatagctactcataacttgctactttctcataaggcggcggttgaagtctacaggaaaaactttcaaaagtgtcaaggtggcgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 101 - DNA, 1623 bp, Artificial sequence
  CRRR
  misc_feature (1)..(1623): CRRR
atgggcagcaaagatgatcagagtttagtagttgcgatatctccagctgctgaaccaaacggaaatcatagtgtgcccattccatttgcttaccctagcatcccaatccagccaagaaaacataataaaccaatagttcatagaagagattttccatcagacttcatcctaggagctggaggcagtgcgtatcagtgtgaaggtgcatataacgaaggtaatagagggccgtcaatttgggatactttcacaaaccgttaccctgcgaagatagcagatggcagtaatggcaatcaagccatcaactcttacaatttgtacaaggaagacattaaaataatgaaacaaaccgggcttgaaagttatagattttccatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 102 - DNA, 1599 bp, Artificial sequence
  RCRR
  misc_feature (1)..(1599): RCRR
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagaggcccgtcaatttgggatactttcacacaacgtagccccgctaagatttcagatggaagcaacgggaatcaggctataaactgctatcacatgtacaaagaagatataaagattatgaaacaaactggcttagaatcatatagattttccatttcttggtctagagttttaccaggaggtaaccttagcggaggcgttaataaggatggagtgaagttttatcatgacttcatcgacgaactgctggctaatggtatcaaaccatttgctacgctgtttcactgggacctaccacaggctttggaagatgagtacggtggtttcttatctgacagaattgtcgaagattttactgaatatgctgaattttgtttctgggaatttggagacaaagtaaaattctggaccacgtttaatgaaccccatacttatgtagcgagcggttacgcaactggagaatttgctcctggaagagggggcgccgatggaaaaggcaacccaggtaaggaaccatacatagctactcataacttgctactttctcataaggcggcggttgaagtctacaggaaaaactttcaaaagtgtcaaggtggcgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 103 - DNA, 1629 bp, Artificial sequence
  CRRC
  misc_feature (1)..(1629): CRRC
atgggcagcaaagatgatcagagtttagtagttgcgatatctccagctgctgaaccaaacggaaatcatagtgtgcccattccatttgcttaccctagcatcccaatccagccaagaaaacataataaaccaatagttcatagaagagattttccatcagacttcatcctaggagctggaggcagtgcgtatcagtgtgaaggtgcatataacgaaggtaatagagggccgtcaatttgggatactttcacaaaccgttaccctgcgaagatagcagatggcagtaatggcaatcaagccatcaactcttacaatttgtacaaggaagacattaaaataatgaaacaaaccgggcttgaaagttatagattttccatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaatgttaaggggtttttcgtctggtcttttttcgataatttcgagtggaatttggggtatatttgcagatatggtattatccatgttgattataaaactttccaaagatatccgaaagactcagccatttggtacaagaattttatctctgagggattcgtaaccaacactgctaaaaagaggtttagagaagaggataagttggtcgagctagttaagaagcaaaagtattaa

SEQ ID NO: 104 - DNA, 1605 bp, Artificial sequence
  RRRC
  misc_feature (1)..(1605): RRRC
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagaggcccgtcaatttgggatactttcacacaacgtagccccgctaagatttcagatggaagcaacgggaatcaggctataaactgctatcacatgtacaaagaagatataaagattatgaaacaaactggcttagaatcatatagattttccatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaatgttaaggggtttttcgtctggtcttttttcgataatttcgagtggaatttggggtatatttgcagatatggtattatccatgttgattataaaactttccaaagatatccgaaagactcagccatttggtacaagaattttatctctgagggattcgtaaccaacactgctaaaaagaggtttagagaagaggataagttggtcgagctagttaagaagcaaaagtattaa

SEQ ID NO: 105 - DNA, 1605 bp, Artificial sequence
  RCRC
  misc_feature (1)..(1605): RCRC
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagaggcccgtcaatttgggatactttcacacaacgtagccccgctaagatttcagatggaagcaacgggaatcaggctataaactgctatcacatgtacaaagaagatataaagattatgaaacaaactggcttagaatcatatagattttccatttcttggtctagagttttaccaggaggtaaccttagcggaggcgttaataaggatggagtgaagttttatcatgacttcatcgacgaactgctggctaatggtatcaaaccatttgctacgctgtttcactgggacctaccacaggctttggaagatgagtacggtggtttcttatctgacagaattgtcgaagattttactgaatatgctgaattttgtttctgggaatttggagacaaagtaaaattctggaccacgtttaatgaaccccatacttatgtagcgagcggttacgcaactggagaatttgctcctggaagagggggcgccgatggaaaaggcaacccaggtaaggaaccatacatagctactcataacttgctactttctcataaggcggcggttgaagtctacaggaaaaactttcaaaagtgtcaaggtggcgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaatgttaaggggtttttcgtctggtcttttttcgataatttcgagtggaatttggggtatatttgcagatatggtattatccatgttgattataaaactttccaaagatatccgaaagactcagccatttggtacaagaattttatctctgagggattcgtaaccaacactgctaaaaagaggtttagagaagaggataagttggtcgagctagttaagaagcaaaagtattaa

SEQ ID NO: 106 - DNA, 1629 bp, Artificial sequence
  CCRC
  misc_feature (1)..(1629): CCRC
atgggcagcaaagatgatcagagtttagtagttgcgatatctccagctgctgaaccaaacggaaatcatagtgtgcccattccatttgcttaccctagcatcccaatccagccaagaaaacataataaaccaatagttcatagaagagattttccatcagacttcatcctaggagctggaggcagtgcgtatcagtgtgaaggtgcatataacgaaggtaatagagggccgtcaatttgggatactttcacaaaccgttaccctgcgaagatagcagatggcagtaatggcaatcaagccatcaactcttacaatttgtacaaggaagacattaaaataatgaaacaaaccgggcttgaaagttatagattttccatttcttggtctagagttttaccaggaggtaaccttagcggaggcgttaataaggatggagtgaagttttatcatgacttcatcgacgaactgctggctaatggtatcaaaccatttgctacgctgtttcactgggacctaccacaggctttggaagatgagtacggtggtttcttatctgacagaattgtcgaagattttactgaatatgctgaattttgtttctgggaatttggagacaaagtaaaattctggaccacgtttaatgaaccccatacttatgtagcgagcggttacgcaactggagaatttgctcctggaagagggggcgccgatggaaaaggcaacccaggtaaggaaccatacatagctactcataacttgctactttctcataaggcggcggttgaagtctacaggaaaaactttcaaaagtgtcaaggtggcgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaatgttaaggggtttttcgtctggtcttttttcgataatttcgagtggaatttggggtatatttgcagatatggtattatccatgttgattataaaactttccaaagatatccgaaagactcagccatttggtacaagaattttatctctgagggattcgtaaccaacactgctaaaaagaggtttagagaagaggataagttggtcgagctagttaagaagcaaaagtattaa

SEQ ID NO: 107 - DNA, 1596 bp, Artificial sequence
  VVRR
  misc_feature (1)..(1596): VVRR
atggaatccaaccaaggagagcctctggttgtagcaatcgtaccaaagcctaacgcgtctactgagcaaaaaaactcccatttgattccggcgacaaggtctaaaatcgtcgtccacaggcgtgacttccctcaagattttgtatttggtgcgggagggtctgcgtaccaatgtgaaggggcatacaatgaaggtaatcgtggcccatcaatctgggacacatttacacagaggacaccagctaaaatctcagacggatcaaatggaaaccaagctattaactgttaccacatgtataaggaagacataaagataatgaaacaggccggactggaggcgtaccgtttcagcatctcatggtctagggttctaccgggcggtagattagcagccggagttaataaggatggggtgaagttttatcacgacttcatcgacgaattgctggctaatgggattaagccgttcgccactttgttccactgggatttaccgcaagccttagaagacgagtacggtggtttcttaagccatcgtattgttgacgatttttgtgagtatgcagagttttgtttctgggaatttggcgacaaaattaaatactggactacttttaatgagccacatacattcacagctaacggctacgctctgggggaatttgctcccggtagaggtaaaaatggcaagggcgacccagccacagaaccgtatctggttactcacaatattttactggcccataaagccgccgtagaggcttaccgtaataagttccaaaaatgccaggaaggcgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgacggcgttaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 108 - protein, 542 aa, Artificial sequence
  CRRC
  PEPTIDE (1)..(542): CRRC
MGSKDDQSLVVAISPAAEPNGNHSVPIPFAYPSIPIQPRKHNKPIVHRRDFPSDFILGAGGSAYQCEGAYNEGNRGPSIWDTFTNRYPAKIADGSNGNQAINSYNLYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGFFVWSFFDNFEWNLGYICRYGIIHVDYKTFQRYPKDSAIWYKNFISEGFVTNTAKKRFREEDKLVELVKKQKY

(19) United States
(12) Patent Application Publication
(10) Pub. No.: US 2022/0228180 A1
(43) Pub. Date: Jul. 21, 2022

(54) Methods for production of strictosidine aglycone and monoterpenoid indole alkaloids

(71) Applicant: DANMARKS TEKNISKE UNIVERSITET, Kgs. Lyngby (DK)

(72) Inventors: Michael Krogh Jensen, Copenhagen (DK); Jay D. Keasling, Berkeley, CA (US); Jie Zhang, Birkerød (DK); Lea Gram Hansen, Brønshøj (DK)

(73) Assignee: DANMARKS TEKNISKE UNIVERSITET, Kgs. Lyngby (DK)

(21) Appl. No.: 17/610,224

(22) PCT Filed: May 13, 2020
(86) PCT No.: PCT/EP2020/063283

§ 371 Date: Nov. 10, 2021

Related U.S. Application Data
(60) Provisional application No. 62/846,820, filed on May 13, 2019.

(30) Foreign Application Priority Data
May 22, 2019 (EP) 19175969.5

(51) Int. Cl. C12P 17/18; C12N 15/113; C12N 9/88; C12N 9/24

(52) U.S. Cl. CPC C12P 17/182; C12N 15/113; C12Y 302/01105; C12N 9/2402; C12Y 403/03002; C12N 9/88

(57) ABSTRACT

Herein are provided microbial factories, in particular yeast factories, for production of strictosidine aglycone and optionally other plant-derived compounds. Also provided are methods for producing strictosidine aglycone in a microorganism, as well as useful nucleic acids, vectors and host cells.

### TECHNICAL FIELD

The present invention relates to microbial factories, such as microorganism factories in particular yeast factories and bacterial factories, for production of strictosidine aglycone and optionally other plant-derived compounds. Also provided are methods for producing strictosidine aglycone in a microorganism, as well as useful nucleic acids, vectors and host cells.

### BACKGROUND

Plants produce some of the most potent human therapeutics and have been used for millennia to treat illnesses. Despite the large repertoire of plant-derived pharmaceuticals, most of these products do not make it to the market because they are found in minute quantities in plants, they are difficult to extract, and there is limited knowledge about their biosynthetic pathways.

Furthermore, sourcing plant-derived pharmaceuticals based on plant-based extraction threatens to cause species extinction. New regulatory laws seek to create conditions to promote biodiversity conservation and sustainable use of genetic resources, which in the short term are expected to further affect the supply chains of many valuable plant natural products.

Moreover, many plant species are not readily genetically manipulated, and synthetic chemistry holds little promise for bulk production of complex plant-derived therapeutics. Together, supporting a need for refactored biosynthesis of new and existing pharmaceuticals, in genetically tractable and sustainable production hosts.

The monoterpenoid indole alkaloids (MIAs) are plant secondary metabolites that show a remarkable structural diversity and pharmaceutically valuable biological activities, such as anti-cancer and anti-psychosis properties. The productions of these alkaloids occurs through highly complicated pathways.

The common precursors for the different MIAs are strictosidine, and its deglycosylated form, strictosidine aglycone. Strictosidine is formed by the coupling of secologanin to tryptamine in a reaction catalysed by the enzyme strictosidine synthase. Strictosidine alglycone is natively produced from hydrolyzing strictosidine by strictosidine-beta-glucosidase (SGD). Over 2,000 MIAs can be produced from strictosidine aglycone.

To enable a sustainable supply of therapeutic MIAs, researchers have for decades attempted to elucidate the biosynthetic pathways from MIA producing plants, including both the platform biosynthetic route to the common MIA precursor strictosidine and the anti-cancer drug vinblastine. Moreover, the platform biosynthetic route from geraniol to strictosidine, and the seven-step biosynthetic pathway from tabersonine to vindoline, the immediate precursor of vinblastine has also been refactored in yeast cell factories.

Current methods for production of strictosidine aglycone are mostly based on chemical synthesis or plant extraction. Such methods are not cost-effective and also have a significant impact on the environment. Therefore, methods for cost-effective and environmental-friendly production of strictosidine aglycone are required.

### SUMMARY

The invention concerns a microorganism capable of producing strictosidine aglycone and methods for strictosidine aglycone and monoterpenoid indole alkaloids (MIAs) production in a microorganism.

In one aspect is provided a microorganism capable of producing strictosidine aglycone, said microorganism expresses
a strictosidine-beta-glucosidase (SGD), capable of converting strictosidine to strictosidine aglycone,

wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,

and/or;

wherein said SGD is a mosaic SGD, wherein said mosaic SGD comprises an amino acid sequence having the general formula

D₁-D₂-D₃-D₄

wherein D₁ is a first amino acid sequence from a first SGD,

wherein D₂ is a second amino acid sequence from a second SGD,

wherein D₃ is a third amino acid sequence comprising or consisting of amino acids of SEQ ID NO:91 or a variant thereof having at least 90% identity to SEQ ID NO: 91,

wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

Also provided herein are methods for producing strictosidine aglycone in a microorganism, comprising the steps of:
a) providing a microorganism, said cell expressing:
   a strictosidine-beta-glucosidase (SGD), capable of converting strictosidine to strictosidine aglycone;
b) incubating said microorganism in a medium comprising strictosidine or a substrate which can be converted to strictosidine by said microorganism;
c) optionally, recovering the strictosidine aglycone;
d) optionally, further converting the strictosidine aglycone to monoterpenoid indole alkaloids,

wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,

and/or;

wherein said SGD is a mosaic SGD, wherein said mosaic SGD comprises an amino acid sequence having the general formula

D₁-D₂-D₃-D₄

wherein D₁ is a first amino acid sequence from a first SGD,

wherein D₂ is a second amino acid sequence from a second SGD,

wherein D₃ is a third amino acid sequence comprising or consisting of amino acids of SEQ ID NO:91 or a variant thereof having at least 90% identity to SEQ ID NO: 91,

wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

Also provided herein are nucleic acid constructs comprising a sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 1, SEQ ID NO: 2, SEQ ID NO: 3, SEQ ID NO: 4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:100, SEQ ID NO:101, SEQ ID NO:102, SEQ ID NO:103, SEQ ID NO:104, SEQ ID NO:105, SEQ ID NO:106 and/or SEQ ID NO:107.

Also provided are vectors comprising the above nucleic acids, as well as host cells comprising said vectors and/or said nucleic acids.

Also provided is a kit of parts comprising a microorganism as described herein, and/or nucleic acid constructs as described herein, and/or a vector as described herein, and instructions for use.

Also provided is the use of above nucleic acids, vectors or host cells for the production of strictosidine aglycone.

Also provided herein are methods for producing monoterpenoid indole alkaloids (MIAs) in a microorganism, said method comprising the steps of:
a) providing a microorganism capable of converting strictosidine aglycone to tabersonine and/or catharanthine, said cell expressing:
   optionally, a strictosidine synthase (STR);
   a strictosidine-beta-glucosidase (SGD);
   a NADPH-cytochrome P450 reductase (CPR);
   a Cytochrome b5 (CYB5);
   a Geissoschizine synthase (GS);
   a Geissoschizine oxidase (GO);
   a Redox1;
   a Redox2;
   a Stemmadenine O-acetyltransferase (SAT);
   a O-acetylstemmadenine oxidase (PAS);
   a Dehydroprecondylocarpine acetate synthase (DPAS);
   a Tabersonine synthase (TS); and/or
   a Catharanthine synthase (CS);
b) incubating said microorganism in a medium comprising strictosidine or a substrate which can be converted to strictosidine by said microorganism;
c) optionally, recovering the MIAs;
d) optionally, processing the MIAs into a pharmaceutical compound, wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,

and/or;

wherein said SGD is a mosaic SGD, wherein said mosaic SGD comprises an amino acid sequence having the general formula

D₁-D₂-D₃-D₄

wherein D₁ is a first amino acid sequence from a first SGD,

wherein D₂ is a second amino acid sequence from a second SGD,

wherein D₃ is a third amino acid sequence consisting of amino acids of SEQ ID NO:91 or a variant thereof having at least 90% identity to SEQ ID NO: 91,

wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

Also provided herein are strictosidine aglycone, tetrahydroalstonine, heteroyohimbine, rabersonine and/or catharanthine obtained by the method as described herein.

Also provided herein are methods for treating a disorder such as a cancer, arrhythmia, malaria, psychotic diseases, hypertension, depression, Alzheimer's disease, addiction and/or neuronal diseases, comprising administration of a therapeutic sufficient amount of an MIA or a pharmaceutical compound obtained by the as described herein.

### DESCRIPTION OF DRAWINGS

FIG. 1: High-resolution analytical results of tetrahydroalstonine (THA) obtained from LC-MS analysis of yeast cells (Saccharomyces cerevisiae) expressing SGD derived from Catharanthus roseus (CroSGD) alone and in various tagged and CroSGD-fusion versions, as well as SGD from Rauvolfia serpentina (RseSGD).

FIG. 2: Sequence identity among SGD derived from Catharanthus roseus (CroSGD), Rauvolfia serpentina (RseSGD), Rauvolfia verticillata (RveSGD), Gelsemium sempervirens (GseSGD), Camptotheca acuminate (CacSGD), Scedosporium apiospermum (SapSGD), Uncaria tomentosa (UtoSGD) and Glycine soja (GsoSGD). The eight protein sequences were aligned with the t-Coffee web server.

FIG. 3: Biosynthesis of the heteroyohimbine tetrahydroalstonine measured on LC-MS. The production of tetrahydroalstonine (THA) was measured in yeast strains expressing either GsoSGD, CacSGD, CroSGD, UtoSGD, GseSGD, SapSGD, RveSGD or RseSGD The yeast strain GsoSGD was used as a negative control. The p-value represents comparison between the negative control (GsoSGD) and CacSGD, CroSGD or UtoSGD, respectively.

FIG. 4: GFP-tagged CroSGD and RseSGD localization in yeast. A) A yeast cell expressing GFP-CroSGD. B) A yeast cell expressing GFP-RseSGD. The arrows mark the localization of SGD in the yeast cells.

FIG. 5: The biosynthesis of the heteroyohimbine alstonine in yeast cell factories, expressing RseSGD, CroTHAS and GseSBE, is shown in triplicates in FIG. 5. Alastonine was measured by Orbitrap Fusion™ Tribrid™ MS.

FIG. 6: The yeast strain MIA-DC was feed with 0.1 mM of secologanine and 1 mM of tryptamine and the production of tabersonine and catharanthine were measured by LC-MS. A) Catharanthine production, B) Tabersonine production, C) Catharanthine standard, and D) Tabersonine standard.

FIG. 7: The yeast strain MIA-DC was feed with 0.1 mM of secologanine and 1 mM of tryptamine and the concentration levels of tabersonine and catharanthine in MIA-DC and MIA-DA (control) were measured by LC-MS.

FIG. 8: Biosynthesis of the heteroyohimbine tetrahydroalstonine measured on LC-MS. The production of tetrahydroalstonine (THA) was measured in yeast strains expressing either CroSGD, VmiSGD1, AhuSGD, HimSGD2, SinSGD, TelSGD, VunSGD, NsiSGD1, LprSGD, AchSGD1, HsuSGD, MroSGD, RseSGD2, PgrSGD, OpuSGD, HpiSGD, HanSGD1, AchSGD2, HimSGD1, IpeSGD, LsaSGD1, CarSGD, OeuSGD, AchSGD3, CmaSGD, MmySGD, VmiSGD3, IniSGD, or NsiSGD2. The p-value represents a comparison between the negative control (CroSGD) and OeuSGD, AchSGD3, CmaSGD, MmySGD, VmiSGD3, IniSGD, and NsiSGD2.

FIG. 9: Biosynthesis of the heteroyohimbine tetrahydroalstonine measured on LC-MS. The production of tetrahydroalstonine (THA) was measured in yeast strains expressing one of the mosaic SGDs: RRCC-SGD, RCCC-SGD, CCCC-SGD, CRCC-SGD, CRCR-SGD, RRCR-SGD, CCCR-SGD, RCCR-SGD, CRRC-SGD, RRRC-SGD, RCRC-SGD, CCRC-RGD, RCRR-SGD, CRRR-SGD, RRRR-SGD, and CCRR-SGD.

CCCC-SGD and RRRR-SGD are identical to the two wild type sequences CroSGD and RseSGD. The p-value represents comparisons between the negative control (CCCC-SGD/CroSGD) and all SGDs containing CroSGD domain 3: RRCC-SGD, RCCC-SGD, CRCC-SGD, CRCR-SGD, RRCR-SGD, CCCR-SGD and RCCR-SGD. The color indicates the identity of domain 3 and 4: Light grey—RseSGD domain 3 & 4, medium grey—RseSGD domain 3 & CroSGD domain 4, dark grey—CroSGD domain 3 & CroSGD/RseSGD domain 4.

FIG. 10: Biosynthesis of the heteroyohimbine tetrahydroalstonine measured on LC-MS. The production of tetrahydroalstonine (THA) was measured in yeast strains expressing one of the wild type SGDs (UtoSGD, GseSGD, CroSGD, or RveSGD) or one of the engineered SGDs (UURR-SGD, GGRR-SGD, CCRR-SGD, or VVRR-SGD).

FIG. 11: Biosynthesis of the common MIA precursor strictosidine (A) and heteroyohimbine tetrahydroalstonine (B) in E. coli measures by LC-MS. The production of strictosidine and tetrahydroalstonine were measures in bacterial strains expressing either CroSGD or RseSGD. A strain with an empty expression vector was included as a negative control.

FIG. 12: Multiple sequence alignment of SGDs proteins derived from Catharanthus roseus (CroSGD), Rauvolfia serpentina (RseSGD and RseSGD2), Rauvolfia verticillata (RveSGD), Gelsemium sempervirens (GseSGD), Camptotheca acuminate (CacSGD), Scedosporium apiospermum (SapSGD), Uncaria tomentosa (UtoSGD), Glycine soja (GsoSGD), Vinca minor (VmiSGD1 and VmiSGD3), Tabernaemontana elegans (TeISGD), Amsonia hubrichtii (AhuSGD), Ophiorrhiza pumila, (OpuSGD), Nyssa sinensis, (NsiSGD1 and NsiSGD2), Coffea arabica (CarSGD), Carapichea ipecacuanha (IpeSGD), Handroanthus impetiginosus (HimSGD2 and HimSGD1), Sesamum indicum (SinSGD), Olea europaea (OeuSGD), Actinidia chinensis var. chinensis (AchSGD1, AchSGD2 and AchSGD3), Helianthus annuus (HanSGD), Lactuca sativa (LseSGD), Ipomoea nil (IniSGD), Chelidonium majus (CmaSGD), Vigna unguiculata (VunSGD), Heliocybe sulcate (HsuSGD), Pyricularia grisea (PgrSGD), Lomentospora prolificans (LprSGD), Hydnomerulius pinastri MD-312 (HpiSGD), Madurella mycetomatis (MmySGD), and Moniliophthora roreri MCA 2997 (MroSGD). The protein sequences were aligned with the t-Coffee web server.

FIG. 13: Pairwise sequence identities among the 36 SGD protein sequences aligned in FIG. 8. The pairwise sequence identities were calculated from the alignment with CLC Main Workbench 8.

### DETAILED DESCRIPTION

The present disclosure relates to microorganisms and method for production of strictosidine aglycone and monoterpenoid indole alkaloids (MIA). The microorganism may be any non-natural or natural microorganism. By non-natural is meant an engineered microorganism, which comprises one or more genes which are not native to the microorganism. In some aspects of the present invention the microorganism expresses a heterologous SGD, mosaic SGD or variants thereof.

Microorganisms are microscopic organisms that exist as unicellular, multicellular, or cell clusters. Microorganism may be divided into different types such as bacteria, archaea, yeasts, fungi, protozoa, algae, and viruses. Thus, in one embodiment, the microorganism is selected from the group consisting of bacteria, archaea, yeasts, fungi, protozoa, algae, and viruses. In another embodiment, the microorganism is selected from the group consisting of bacteria, archaea, yeasts, fungi, protozoa and algae. In another embodiment, the microorganism is selected from the group consisting of bacteria, archaea, yeasts, fungi, and algae. In another embodiment, the microorganism is selected from the group consisting of bacteria, archaea yeasts and fungi. In another embodiment, the microorganism is selected from bacteria, yeasts and fungi. In another embodiment, the microorganism is selected from bacteria or yeasts. In a preferred embodiment, the microorganism is a bacteria or a yeast.

In some embodiments, the microorganism is a bacteria. In one embodiment, the genus of said bacteria is selected from Escherichia, Corynebacterium, Pseudomonas, Bacillus, Lactococcus, Lactobacillus, Halomonas, Bifidobacterium and Enterococcus. In preferred embodiments, the genus of said bacteria is Escherichia. In another embodiment, the microorganism may be selected from the group consisting of Escherichia, Corynebacterium glutamicum, Pseudomonas putida, Bacillus subtilis, Lactococcus bacillus, Halomonas elongate, Bifidobacterium infantis and Enterococcus faecali. In preferred embodiments, the micororganims is an Escherichia. In some embodiments the bacteria is selected from the group consisting of Escherichia coli, Corynebacterium glutamicum, Pseudomonas putida, Bacillus subtilis, Lactococcus bacillus, Halomonas elongate, Bifidobacterium infantis and Enterococcus faecal

In some embodiments, the microorganism is a yeast. In some embodiments, the microorganism is a cell from a GRAS (Generally Recognized As Safe) organism or a non-pathogenic organism or strain. In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In preferred embodiments, the genus of said yeast is Saccharomyces.

The microorganism may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the microorganism is a Saccharomyces cerevisiae cell.

Microorganism

Herein is thus provided a microorganism capable of producing strictosidine aglycone, said microorganism expresses
a strictosidine-beta-glucosidase (SGD), capable of converting strictosidine to strictosidine aglycone,
wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
and/or;
wherein said SGD is a mosaic SGD, wherein said mosaic SGD comprises an amino acid sequence having the general formula

D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

The microorganismsdisclosed herein are thus all capable of converting strictosidine to strictosidine aglycone, when strictosidine is provided to the microorganism. In some embodiments, strictosidine is provided to the microorganism, for example by feeding strictosidine to the microorganism in the medium. In other embodiments, the microorganism is capable of synthesising strictosidine, for example the microorganism is further engineered as described below.

In another embodiment said microorganism further expresses a strictosidine synthase (STR), capable of converting secologanin and tryptamine to strictosidine. Thus, microorganisms further expressing STR are capable of converting secologanin and tryptamine to strictosidine aglycone, when secologanin and tryptamine are provided to the microorganism. Secologanin and tryptamine may be provided e.g. in the medium. However, in some embodiments the microorganism is capable of synthesising secologanin and/or tryptamine, for example the microorganismis further engineered to synthesis secologanin and/or tryptamine.

Strictosidine-O-beta-D-glucosidase (SGD)

The first heterologous enzyme expressed in the microorganism is capable of converting strictosidine to strictosidine aglycone. The first heterologous enzyme is not natively expressed in the microorganism. It may be derived from a eukaryote or a prokaryote, as detailed below, preferably a eukaryotic cell such as a plant cell.

In some embodiments, the first heterologous enzyme is a strictosidine-O-beta-D-glucosidase, herein also termed SGD, and having an EC number EC 3.2.1.105. This enzyme catalyses the following reaction:

Strictosidine+H₂O<=>D-glucose+strictosidine aglycone.

Heterologous SGD or Variants Thereof

Thus the microorganism expressing the first heterologous enzyme is capable of converting strictosidine to strictosidine aglycone by the action of the first heterologous enzyme.

The conversion of strictosidine to strictosidine aglycone, may be measured directly by the amount of strictosidine aglycone as known in the art, or surrogate measure of the conversion of strictosidine to strictosidine aglycone may be measured as known in the art. Because strictosidine aglycone is highgly reactive, indirect determination of strictosidine aglycone may be preferred. For example, colorimetric assays to follow strictosidine consumption as described in Geerlings et al., 2000, may be used. The disappearance of strictosidine may also be monitored by UV, as described in Guirimand et al., 2010, or the general p-glucosidase activity in the cells may be measured, e.g. by UV detection of a synthetic substrate such as 4-methylumbelliferyl-β-D-glucoside (Guirimand et al., 2010).

Thus, to determine whether a SGD is capable of converting strictosidine to strictosidine aglycone, the person skilled in the art could use any of said methods, or could use high-precision mass spectrometry to detect the accurate mass of strictosidine aglycone after cultivation of a strain expressing an SGD or an enzyme suspected of having SGD activity in a medium; the cell is either provided with strictosidine in the medium or it has been engineered and can synthesise strictosidine. The strictosidine aglycone can be detected directly in the medium or in a pellet, after centrifugation of the culture broth. Alternatively, the appearance of other products, downstream of strictosidine aglycone, for example tetrahydroalstonine, can be monitored; such products will only form in the presence of a functional SGD, strictosidine, and an enzyme capable of using strictosidine aglycone, as described in e.g. Stavrinides et al., 2015.

In some embodiments, the first heterologous enzyme is an SGD which is native to Rauvolfia serpentina, Gelsemium sempervirens, Scedosporium apiospermum or Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 2997 or a functional variant thereof.

In other words, in some embodiments the SGD is derived from Rauvolfia serpentina, Gelsemium sempervirens, Scedosporium apiospermum, Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 2997 or a functional variant thereof. Functional variants of SGD are modified enzymes which retain the capability to convert strictosidine to strictosidine aglycone. In some embodiments, the SGD is RseSGD as set forth in SEQ ID NO: 24 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 24. In other embodiments, the SGD is GseSGD as set forth in SEQ ID NO: 25 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 25. In other embodiments, the SGD is SapSGD as set forth in SEQ ID NO: 26 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 26. In other embodiments, the SGD is RveSGD as set forth in SEQ ID NO: 27 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 27. In other embodiments, the SGD is VmiSGD1 as set forth in SEQ ID NO: 47 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 47. In other embodiments, the SGD is AhuSGD as set forth in SEQ ID NO: 48 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 48. In other embodiments, the SGD is HimSGD2 as set forth in SEQ ID NO: 49 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 49. In other embodiments, the SGD is SinSGD as set forth in SEQ ID NO: 50 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 50. In other embodiments, the SGD is TelSGD as set forth in SEQ ID NO: 51 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 51. In other embodiments, the SGD is VunSGD as set forth in SEQ ID NO: 52 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 52. In other embodiments, the SGD is NsiSGD1 as set forth in SEQ ID NO: 53 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 53. In other embodiments, the SGD is LprSGD as set forth in SEQ ID NO: 54 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 54. In other embodiments, the SGD is AchSGD1 as set forth in SEQ ID NO: 55 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 55. In other embodiments, the SGD is HsuSGD as set forth in SEQ ID NO: 56 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 56. In other embodiments, the SGD is MroSGD as set forth in SEQ ID NO: 57 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 57. In other embodiments, the SGD is RseSGD2 as set forth in SEQ ID NO: 58 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 58. In other embodiments, the SGD is PgrSGD as set forth in SEQ ID NO: 59 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 59. In other embodiments, the SGD is OpuSGD as set forth in SEQ ID NO: 60 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 60. In other embodiments, the SGD is HpiSGD as set forth in SEQ ID NO: 61 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 61. In other embodiments, the SGD is HanSGD1 as set forth in SEQ ID NO: 62 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 62. In other embodiments, the SGD is AchSGD2 as set forth in SEQ ID NO: 63 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 63. In other embodiments, the SGD is HimSGD as set forth in SEQ ID NO: 64 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 64. In other embodiments, the SGD is IpeSGD as set forth in SEQ ID NO: 65 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 65. In other embodiments, the SGD is LsaSGD as set forth in SEQ ID NO: 66 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 66. In other embodiments, the SGD is CarSGD as set forth in SEQ ID NO: 67 or a functional variant thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 67.

Preferably, the SGD is RseSGD or a functional variant thereof.

In some embodiments, the SGD originates from a MIA producing plant species, wherein said SGD shares at least 65% sequence identity to RseSGD. Thus, in some embodiments, the SGD is selected from the group consisting of RseSGD, RveSGD, TelSGD, or VmiSGD or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 24, SEQ ID NO: 27, SEQ ID NO: 51 or SEQ ID NO: 47.

In some embodiments, the SGD originates from a MIA producing plant species, wherein said SGD shares at the most 65% sequence identity to RseSGD. Thus, in some embodiments, the SGD is selected from the group consisting of GseSGD, NsiSGD, OpuSGD, AhuSGD, or RseSGD2 or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 25, SEQ ID NO: 53 SEQ ID NO: 60, SEQ ID NO: 48 or SEQ ID NO: 58.

A person skilled in the art would know how to determine sequence identity between two species by using known methods in the art.

In some embodiments, the SGD originates from a non-MIA producing plant species. Thus, in some embodiments, the SGD is selected from the group consisting of AchSGD1, AchSGD2, CarSGD, HanSGD, HimSGD1, HimSGD2, LsaSGD1, SinSGD, VunSGD or IpeSGD or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 55, SEQ ID NO: 63, SEQ ID NO: 67, SEQ ID NO: 62, SEQ ID NO: 64, SEQ ID NO: 49, SEQ ID NO: 66, SEQ ID NO: 50, SEQ ID NO: 52 or SEQ ID NO: 65.

In some embodiments, the SGD originates from a non-MIA producing fungi species. Thus, in some embodiments, the SGD is selected from the group consisting of HpiSGD, HsuSGD, LprSGD, MroSGD, PgrSGD, or SapSGD or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 61, SEQ ID NO: 56, SEQ ID NO: 54, SEQ ID NO: 57, SEQ ID NO: 59 or SEQ ID NO: 26.

In other embodiments, said microorganism, such as the yeast cell or the bacteria cell, is capable of producing at least 1 μM tetrahydroalstonine. Thus, in some embodiments, the SGD is selected from the group consisting of RseSGD, VmiSGD or AhuSGD, or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 24, SEQ ID NO: 47 or SEQ ID NO: 48.

In other embodiments the SGD is selected from the group consisting of RseSGD, GseSGD, SapSGD or RveSGD, or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 24, SEQ ID NO: 25, SEQ ID NO: 26 or SEQ ID NO: 27.

In other embodiments the SGD is selected from the group consisting of RseSGD, GseSGD, SapSG, RveSGD, VmiSGD, AhuSGD or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 24, SEQ ID NO: 25, SEQ ID NO: 26, SEQ ID NO: 27, SEQ ID NO: 47 or SEQ ID NO: 48.

In other embodiments the SGD is selected from the group consisting of RseSGD, RveSGD, VmiSGD, AhuSGD, HimSGD, SinSGD or TelSGD, or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 24, SEQ ID NO: 27, SEQ ID NO: 47, SEQ ID NO: 48, SEQ ID NO: 49, SEQ ID NO: 50 or SEQ ID NO: 51.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), or LsaSGD1 (SEQ ID NO: 66), or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), RveSGD (SEQ ID NO: 27), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), SapSGD (SEQ ID NO: 26), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), GseSGD (SEQ ID NO: 25), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from RseSGD (SEQ ID NO: 24), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In some embodiments, said SGD is selected from GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

Thus, in some embodiments the microorganism according to the present invention may express a SGD as described herein above. In other embodiments, the microorganism according to the present invention may express a mosaic SGD. The microorganism may be a yeast cell or a bacteria cell, as described herein.

Mosaic SGD or Variants Thereof

The inventors have engineered new and active mosaic SGDs capable of converting strictosidine into strictosidine aglycone. Said mosaic SGDs are useful in microorganism factories, such as yeast factories and bacteria factories, for production of strictosidine aglycone, tetrahydroalstonine and/or other MIA products.

Thus, the present invention also relates to a mosaic SGD, wherein said mosaic SGD comprises an amino acid sequence having the general formula

D₁-D₂-D₃-D₄

wherein D₁ is a first amino acid sequence from a first SGD,

wherein D₂ is a second amino acid sequence from a second SGD,

wherein D₃ is a third amino acid sequence comprising or consisting of amino acids of SEQ ID NO:91 or a variant thereof having at least 90% identity to SEQ ID NO: 91,

wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

The mosaic SGD thus comprises at least one domain of RseSGD, namely the third domain D₃, and at least one other domain as defined above which is not a domain of RseSGD.

The inventors found that a SGD can be divided into four domains:
Domain 1 (D₁)
Domain 2 (D₂)
Domain 3 (D₃)
Domain 4 (D₄)

Examples hereof are described in Examples 8 and 9 herein below.

Each of domain 1-4 consists of a consecutive sequence of amino acids. Domain 1 is the most N-terminal amino acid sequence in the SGD. The first amino acid residue in domain 1 is typically methionine, as this is the first amino acid which is translated from a start codon, however it may occur that the first domain actually starts with another residue in embodiments where part of the domain would be cleaved off, thereby removing the methionine. Being the first domain in SGD, domain 1 is followed by domain 2, which is followed by domain 3, which is followed by domain 4. Domain 4 is the most C-terminal amino acid sequence in the SGD. The last amino acid residue in domain 4 is the last amino acid residue in the consecutive sequence of the SGD.

The positions of the amino acids in each domain 1-4 of a SGD may be defined by aligning the SGD amino acid sequence to the amino acid sequence RseSGD of SEQ ID NO:24, hereby using RseSGD as a reference sequence. Thus, is it to be understood that following alignment between a SGD amino acid sequence and the reference amino acid sequence of SEQ ID NO:24, an amino acid corresponds to position X of SEQ ID NO:24 if it aligns to the same position.

For example, the domains can be defined as follows. Starting from an SGD which is not RseSGD, and which hereinafter is termed XxxSGD, a pairwise alignment of the two amino acid sequences of RseSGD and XxxSGD is performed to determine the boundaries of the domains in XxxSGC.

Domain 1 in XxxSGD can thus be defined as follows. Domain 1 of RseSGD (as set forth in SEQ ID NO: 89) is used to align XxxSGD. The first domain is then defined as the region of XxxSGD starting with the amino acid that aligns with the first residue of SEQ ID NO: 89 and finishing with the amino acid that aligns with the last residue of SEQ ID NO: 89. In embodiments where this amino acid is not a methionine, the introduction of a methionine immediately upstream of this first domain may be necessary in order to ensure proper translation of the protein, as is known in the art.

The same procedure can be repeated for domains 2 and 3, as needed. Domain 2 in XxxSGD can thus be defined as follows. Domain 2 of RseSGD (as set forth in SEQ ID NO: 90) is used to align XxxSGD. The second domain is then defined as the region of XxxSGD starting with the amino acid that aligns with the first residue of SEQ ID NO: 90 and finishing with the amino acid that aligns with the last residue of SEQ ID NO: 90. Domain 3 in XxxSGD can thus be defined as follows. Domain 3 of RseSGD (as set forth in SEQ ID NO: 91) is used to align XxxSGD. The third domain is then defined as the region of XxxSGD starting with the amino acid that aligns with the first residue of SEQ ID NO: 91 and finishing with the amino acid that aligns with the last residue of SEQ ID NO: 91. The third domain of the mosaic SGD is domain D₃ of RseSGD as set forth in SEQ ID NO: 91, but it may still be useful to determine the position of domain 3 in XxxSGD, particularly in order to determine the position of domain 4 in XxxSGD.

Domain 4 in XxxSGD preferably corresponds to the region starting with the first amino acid immediately downstream of domain 3 of the same XxxSGD and finishing with the last amino acid of XxxSGD. In other words, if domain 3 of XxxSGD ends with residue number n, then domain 4 starts with residue n+1, where n is an integer.

The term “domain 1” as used herein refers to one or more sequential groups of amino acids corresponding to amino acids from position 1 to 115 of SEQ ID NO:24.

The term “domain 2” as used herein refers to one or more sequential groups of amino acids corresponding to amino acids from position 116 to 266 of SEQ ID NO:24.

The term “domain 3” as used herein refers to one or more sequential groups of amino acids corresponding to amino acids from position 267 to 456 of SEQ ID NO:24.

The term “domain 4” as used herein refers to one or more sequential groups of amino acids corresponding to amino acids from position 457 to 532 of SEQ ID NO:24.

The four domains of the mosaic SGD may be linked by, or separated by, small sequences, for example amino acid linkers, as is known in the art. It will thus be understood that the mosaic SGD may comprise additional amino acids which can be added to each of the four domains, as is known in the art.

In some embodiments, the mosaic SGD may be further modified, for example by the introduction of additional domains which may increase the stability or longevity or half-life of the protein, or localidation domains targeting the mosaic SGD to specific cellular localisations. Relevant additional domains are known in the art.

A non-functional SGD as used herein referes to a SGD which is not capable of converting strictosidine to strictosidine aglycone, whereas in contrast, a functional SGD is capable of converting strictosidine to strictosidine aglycone. By introducing some domains of RseSGD into a non-functional SGD however, it may be possible to restore function of a non-functional SGD, as shown in the examples, thus obtaining a functional mosaic SGD.

In some embodiments, D₁ is a first amino acid sequence from a first SGD. Said first SGD may be any SGD, such as a functional or a non-functional SGD. It is preferred that said first SGD has at least 70%, such as at least 75%, such as at least 80%, such as at least 85%, such as at least 90%, such as at least 95% identity to RseSGD of SEQ ID NO: 24.

In some embodiments, D₂ is a second amino acid sequence from a second SGD. Said second SGD may be any SGD, such as a functional or a non-functional SGD. It is preferred that said second SGD has at least 70%, such as at least 75%, such as at least 80%, such as at least 85%, such as at least 90%, such as at least 95% identity to RseSGD of SEQ ID NO: 24.

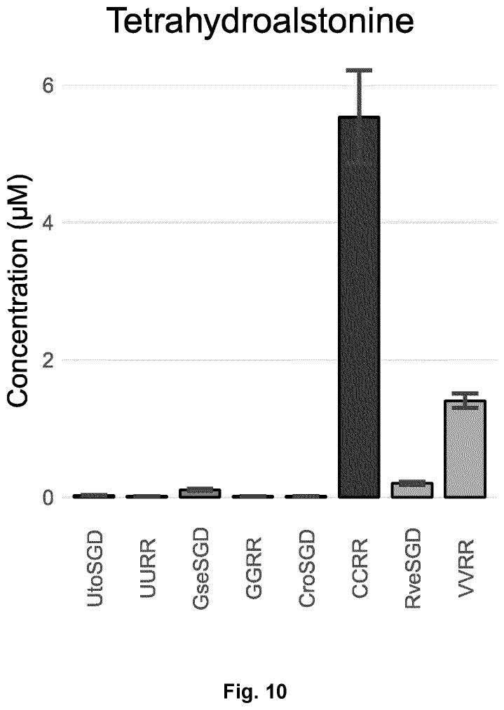

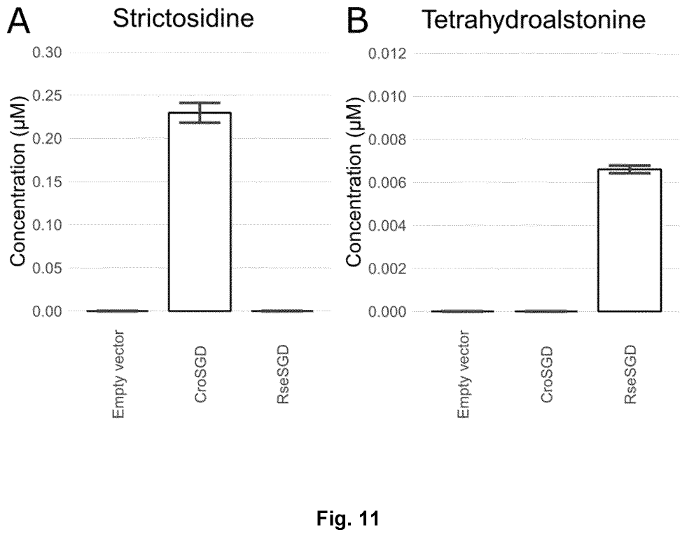

Interestingly, the inventors found that domain 3 (D₃) of RseSGD consisting of an amino acid sequence of SEQ ID NO:91 is capable of rescuing the inability of a non-functional SGDs of converting strictosidine to strictosidine aglycone (see FIGS. 9 and 10). Thus in preferred embodiments, the mosaic SGD comprises 4 domains, of which at least one comprises or consists of domain 3 of RseSGD; this domain is set forth in SEQ ID NO: 91.

Thus, in some embodiments of the present invention, the mosaic SGD comprises a D_{3,} wherein said D₃ is a third amino acid sequence consisting of amino acids of SEQ ID NO:91 or a variant thereof having at least 70%, such as at least 75%, such as at least 80%, such as at least 85%, such as at least 90% identity to SEQ ID NO: 91. In other words, said D₃ is an amio acid sequence of domain 3 of RseSGD.

In some embodiments, D₄ is a fourth amino acid sequence from a fourth SGD or an amino acid sequence consisting of amino acids of SEQ ID NO:92 or a variant thereof having at least 70%, such as at least 75%, such as at least 80%, such as at least 85%, such as at least 90% identity to SEQ ID NO: 92. Said fourth SGD may be any SGD, such as a functional or a non-functional SGD. It is preferred that said fourth SGD has at least 70%, such as at least 75%, such as at least 80%, such as at least 85%, such as at least 90%, such as at least 95% identity to RseSGD of SEQ ID NO: 24.

In a preferred embodiment, said mosaic SGD comprises a D_{4,} wherein said D₄ is a fourth amino acid sequence consisting of amino acids of SEQ ID NO:92 or a variant thereof.

Said first SGD, second SGD and fourth SGD can be the same or different, with the proviso that said first SGD, second SGD and fourth SGD are not all RseSGD. In other words, said mosaic SGD may not be an RseSGD of SEQ ID NO: 24. Thus, said first first SGD, second SGD and fourth SGD, may be of the same species or different species, however said first first SGD, second SGD and fourth SGD may not all be native to Rauvolfia serpentina.

The third domain of the mosaic SGD comprises or consists of the third domain of RseSGD as detailed above, and at least one of the first domain, the second domain and the fourth domain is from a second organism which is not Rauvolfia serpentina, for example at least one of D₁, D₂ or D₄ is from an SGD native to an organism selected from Gelsemium sempervirens, Scedosporium apiospermum or Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 2997 or a variant thereof—as explained above, the variant here does not need to be functional to begin with, as its activity may be rescued by the D₃ domain of RseSGD.

In some embodiments, each of D₁, D₂ and D₄ are from different SGDs, and are derived from different organisms independently selected from the group consisting of Scedosporium apiospermum, Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 299. In such embodiments, one of D₁, D₂ and D₄ may be D₁, D₂ or D₄ from RseSGD as set forth in SEQ ID NO: 89, SEQ ID NO: 90 or SEQ ID NO: 92, respectively, or variants thereof having at least 70% identity or homology thereto.

In some embodiments, two of D₁, D₂ and D₄ are from the same SGD, and are derived from one organism and the remaining domain is from another SGD. Relevant organisms and SGDs have been described above in the section “ Strictosidine-O-beta-D-glucosidase”. For example, D₁ and D₂ are from one SGD from a first organism, and

D₄ is from another SGD from another organism; or D₁ and D₄ are from one SGD from a first organism, and D₂ is from another SGD from another organism; or D₂ and D₄ are from one SGD from a first organism, and D₁ is from another SGD from another organism, which may be Rauvolfia serpentina. The first organism and the other organism may be different organisms which are independently selected from the group consisting of Scedosporium apiospermum, Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 299.

In some embodiments, all of D₁, D₂ and D₄ are from the same SGD of the same organism, which is not Rauvolfia serpentina. D₁, D₂ and D₄ may be of an SGD native to an organism selected from the group consisting of Scedosporium apiospermum, Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 299.

Thus in some embodiments, the first, second and fourth SGD are all from the same SGD, which is not RseSGD. In other embodiments, the first and second SGD are from the same SGD and the fourth SGD is from another SGD; at least one said two SGDs is not RseSGD. In other embodiments, the first and third SGD are from the same SGD and the fourth SGD is from another SGD; at least one said two SGDs is not RseSGD. In other embodiments, the fourth and second SGD are from the same SGD and the fourth SGD is from another SGD; at least one said two SGDs is not RseSGD. In some embodiments, the first, second and fourth SGD are all from different SGDs, one of which may be RseSGD.

In one embodiment, the mosaic SGD comprises or consists of an amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99 or SEQ ID NO: 108, or variants thereof having at least 90% identity or homology thereto, such as at least 91%, such as at least 92%, such as at least 93%, such as at least 94%, such as at least 95%, such as at least 96%, such as at least 97%, such as at least 98%, such as at least 99% identity or homology thereto.

The SGD may be expressed in the microorganism by introducing a nucleic acid sequence as detailed further below, which encodes a SGD. In particular, the nucleic acid sequence is identical to or has at least 90% identity to SEQ ID NO: 1, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 1. Thus, the microorganism of the invention or the microorganism used in the methods of the invention preferably comprises at least a nucleic acid sequence identical to or having at least 90% identity to SEQ ID NO: 1.

In other embodiments, the nucleic acid sequence is identical to or has at least 90% identity to SEQ ID NO:2, SEQ ID NO:3, SEQ ID NO:4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:100, SEQ ID NO:101, SEQ ID NO:102, SEQ ID NO:103, SEQ ID NO:104, SEQ ID NO:105, SEQ ID NO:106 or SEQ ID NO:107 such as at least 91%, such as at least 92%, such as at least 93%, such as at least 94%, such as at least 95%, such as at least 96%, such as at least 97%, such as at least 98%, such as at least 99%, such as 100% identity to SEQ ID NO:2, SEQ ID NO:3, SEQ ID NO:4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88 SEQ ID NO:100, SEQ ID NO:101, SEQ ID NO:102, SEQ ID NO:103, SEQ ID NO:104, SEQ ID NO:105, SEQ ID NO:106 or SEQ ID NO:107.

As is known in the art, in the event that the first domain of XxxSGD used in the mosaic SGD is not a methionine, the skilled person will readily be able to introduce a start codon in the nucleic acid sequence encoding the mosaic SGD in order to ensure proper translation of the mosaic SGD. The skilled person will also know how to introduce short nucleic acid sequences corresponding to linkers separating the different domains in the mosaic SGD.

The microorganism according to the present invention, expressing a heterologous SGD or variant thereof, and/or a mosaic SGD or variant thereof, is capable of converting strictosidine to strictosidine aglycone.

The conversion of strictosidine to strictosidine aglycone, may be measured directly by the amount of strictosidine aglycone as known in the art, or surrogate measure of the conversion of strictosidine to strictosidine aglycone may be measured as known in the art. Because strictosidine aglycone is highgly reactive, indirect determination of strictosidine aglycone may be preferred. For example, colorimetric assays to follow strictosidine consumption as described in Geerlings et al., 2000, may be used. The disappearance of strictosidine may also be monitored by UV, as described in Guirimand et al., 2010, or the general 8-glucosidase activity in the cells may be measured, e.g. by UV detection of a synthetic substrate such as 4-methylumbelliferyl-β-D-glucoside (Guirimand et al., 2010).

Thus, to determine whether a SGD is capable of converting strictosidine to strictosidine aglycone, the person skilled in the art could use any of said methods, or could use high-precision mass spectrometry to detect the accurate mass of strictosidine aglycone after cultivation of a strain expressing an SGD or an enzyme suspected of having SGD activity in a medium; the cell is either provided with strictosidine in the medium or it has been engineered and can synthesise strictosidine. The strictosidine aglycone can be detected directly in the medium or in a pellet, after centrifugation of the culture broth. Alternatively, the appearance of other products, downstream of strictosidine aglycone, for example tetrahydroalstonine, can be monitored; such products will only form in the presence of a functional SGD, strictosidine, and an enzyme capable of using strictosidine aglycone, as described in e.g. Stavrinides et al., 2015.

Strictosidine Synthase (STR)

Strictosidine may be provided to the microorganism, for example as part of the medium the cell is incubated in. In some embodiments, however, the microorganism is engineered and is capable of synthesising strictosidine from secologanin and tryptamine.

Thus in some embodiments the microorganism expresses a heterologous strictosidine synthase having an EC number EC 4.3.3.2. Such enzymes catalyse a Pictet-Spengler reaction between the aldehyde group of secologanin and the amino group of tryptamine to yield strictosidine.

Thus microorganisms expressing a heterologous STR are capable of converting secologanin and tryptamine to strictosidine.

In some embodiments, the STR is the STR native to Catharanthus roseus or a functional variant thereof which retains the ability to convert secologanin and tryptamine to strictosidine. Thus in some embodiments, the STR is CroSTR as set forth in SEQ ID NO: 30 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 30.

Thus, in some embodiments, the microorganism expresses RseSGD as set forth in SEQ ID NO: 24 and CroSTR as set forth in SEQ ID NO: 30, or functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity thereto. In some embodiments, the microorganism expresses GseSGD as set forth in SEQ ID NO: 25 and CroSTR as set forth in SEQ ID NO: 30, or functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity thereto. In some embodiments, the microorganism expresses SapSGD as set forth in SEQ ID NO: 26 and CroSTR as set forth in SEQ ID NO: 30, or functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity thereto. In some embodiments, the microorganism expresses RveSGD as set forth in SEQ ID NO: 27 and CroSTR as set forth in SEQ ID NO: 30, or functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

The STR may be expressed in the microorganism by introducing a nucleic acid sequence as detailed further below, which encodes an STR. In particular, the nucleic acid sequence is identical to or has at least 90% identity to SEQ ID NO: 7, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 7.

Tetrahydroalstonine Synthase, Heteroyohimbine Synthase

In addition to the above, the microorganism may be further engineered so that it can produce tetrahydroalstonine.

In some embodiments, the microorganism expresses an SGD and optionally an STR, and further expresses a heterologous tetrahydroalstonine synthase (THAS), which is not natively present in the cell. Tetrahydroalstonine synthase has an EC number EC 1.-.-.- and catalyses conversion of strictosidine aglycone to tetrahydroalstonine. The microorganism when expressing a THAS is thus able to convert strictosidine aglycone to tetrahydroalstonine, thus producing tetrahydroalstonine.

In some embodiments, the microorganism expresses an SGD and optionally an STR, and further expresses a heteroyohimbine synthase (HYS), which is not natively present in the cell. Heteroyohimbine synthase has an EC number EC 1.-.-.- and catalyses conversion of strictosidine aglycone to tetrahydroalstonine, ajmalicine, or mayumbine.

The microorganism when expressing an HYS is thus able to convert strictosidine aglycone to tetrahydroalstonine, ajmalicine, or mayumbine, thus producing tetrahydroalstonine.

In some embodiments, the microorganism expresses a SGD and optionally an STR and further expresses a THAS and an HYS.

In preferred embodiments, the THAS is the THAS native to Catharanthus roseus or a functional variant thereof which retains the ability to convert strictosidine aglycone to tetrahydroalstonine. Thus in some embodiments, the THAS is CroTHAS as set forth in SEQ ID NO: 28 or a functional variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 28.

The THAS may be expressed in the microorganism by introducing a nucleic acid sequence as detailed further below, which encodes a THAS. In particular, the nucleic acid sequence is identical to or has at least 90% identity to SEQ ID NO: 5, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 5.

In other preferred embodiments, the HYS is the HYS native to Catharanthus roseus or a functional variant thereof which retains the ability to convert strictosidine aglycone to tetrahydroalstonine, ajmalicine, or mayumbine. Thus in some embodiments, the HYS is CroHYS as set forth in SEQ ID NO: 46 or variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 46.

The HYS may be expressed in the microorganism by introducing a nucleic acid sequence as detailed further below, which encodes an HYS. In particular, the nucleic acid sequence is identical to or has at least 90% to SEQ ID NO: 23, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 23.

In some embodiments, the microorganism expresses CroHYS and/or CroTHAS or functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 46 and/or SEQ ID NO: 28.

The microorganism expressing THAS and/or HYS further expresses an SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Sarpargan Bridge Enzyme (SBE)

In addition to the above, the microorganism may be further engineered so that it can produce a heteroyohimbine, in particular alstonine and serpentine. Heteroyohimbines are a prevalent subclass of the monoterpene indole alkaloids, which are found in many plant species, primarily from the Apocynaceae and Rubiaceae families. Examples of heteroyohimbines include the al-adrenergic receptor antagonist ajmalicine, and the benzodiazepine receptor ligand mayumbine (19-epi-ajmalicine). Oxidized β-carboline heteroyohimbines also exhibit potent pharmacological activity: serpentine has shown topoisomerase inhibition activity and alstonine has been shown to interact with 5-HT2A/C receptors and may act as an anti-psychotic agent. In addition, heteroyohimbines are biosynthetic precursors of many oxindole alkaloids, which also display a wide range of biological activities.

In some embodiments, the microorganism expresses an SGD and optionally an STR, and further expresses a heterologous sarpargan bridge enzyme (SBE), which is not natively present in the cell. This enzyme has an EC number EC 1.14.14.- and catalyses conversion of tetrahydroalstonine and ajmalicine to the corresponding alstonine and serpentine, respectively, or converts by cyclization the strictosidine-derived geissoschizine to the sarpagan alkaloid polyneuridine aldehyde. The microorganism when expressing an SBE is thus able to convert tetrahydroalstonine to alstonine and serpentine. In embodiments where the cell is capable of producing ajmalicine, the microorganism when expressing an SBE is able to convert tetrahydroalstonine and ajmalicine to alstonine and serpentine.

In preferred embodiments, the SBE is the SBE native to Gelsemium sempervirens or a functional variant thereof which retains the ability to convert tetrahydroalstonine and ajmalicine to alstonine and serpentine. Thus in some embodiments, the SBE is GseSBE as set forth in SEQ ID NO: 29 or a functional variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 29.

The SBE may be expressed in the microorganism by introducing a nucleic acid sequence as detailed further below, which encodes an SBE. In particular, the nucleic acid sequence is identical to or has at least 90% identity to SEQ ID NO: 6, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 6.

The microorganism also expresses a SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

The microorganism may also express a THAS and/or an HYS as described herein, in particular the microorganism expresses CroHYS and/or CroTHAS or functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 46 and SEQ ID NO: 28.

NADPH-Cytochrome P450 Reductase, Cytochrome b5 and Geissoschizine Synthase

The microorganism may be further engineered so that it can produce 19E-geissoschizine.

In some embodiments, the microorganism expresses an SGD and optionally an STR, and further expresses a heterologous NADPH-cytochrome P450 reductase (CPR), a heterologous Cytochrome b5 (CYB5) and a heterologous Geissoschizine synthase (GS) which are not natively present in the microorganism. NADPH-cytochrome P450 reductase has an EC number EC 1.6.2.4 and is required for electron transfer from NADP to cytochrome P450. Cytochrome b5 has an EC number EC 1.6.2.2 and is a membrane bound hemoprotein which function as an electron carrier. Geissoschizine synthase has an EC number EC 1.3.1.36 and catalyzes the reduction of strictosidine aglycone to 19E-geissoschizine. The microorganism when expressing CPR, CYB5 and GS is thus able to convert strictosidine aglycone to 19E-geissoschizine, thus producing 19E-geissoschizine.

In some embodiments, the microorganism expresses an SGD and optionally an STR and further expresses CPR, CYB5 and GS.

In preferred embodiments, the CPR is the CPR native to Catharanthus roseus or a functional variant thereof which retains the ability to transfer electrons from NADP to cytochrome P450. Thus in some embodiments, the CPR is CroCPR as set forth in SEQ ID NO: 31 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 31.

The CPR may be expressed in the microorganism by introducing a nucleic acid sequence as detailed further below, which encodes a CPR. In particular, the nucleic acid sequence is identical to or has at least 90% identity to SEQ ID NO: 8, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 8.

In preferred embodiments, the CYB5 is the CYB5 native to Catharanthus roseus or a functional variant thereof which retains the ability to function as an electron carrier. Thus in some embodiments, the CYB5 is CroCYB5as set forth in SEQ ID NO: 32 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 32.

The CYB5 may be expressed in the microorganism by introducing a nucleic acid sequence as detailed further below, which encodes a CYB5. In particular, the nucleic acid sequence is identical to or has at least 90% identity to SEQ ID NO: 9, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 9.

In preferred embodiments, the GS is the GS native to Catharanthus roseus or a functional variant thereof which retains the ability to catalyze the reduction of strictosidine aglycone to 19E-geissoschizine. Thus in some embodiments, the GS is CroGS as set forth in SEQ ID NO: 33 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 33.

The GS may be expressed in the microorganism by introducing a nucleic acid sequence as detailed further below, which encodes a GS. In particular, the nucleic acid sequence is identical to or has at least 90% identity to SEQ ID NO: 10, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 10.

The microorganism further expresses an SGD as described herein, in particular RseSGD as set forth in SEQ ID NO: 24, GseSGD as set forth in SEQ ID NO: 25,SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Geissoschizine Oxidase, Redox1 and Redox2

The microorganism may be further engineered so that it can produce stemmadenine.

The microorganism may be as described herein above. In some embodiments, the microorganism is a yeast cell. In other embodiments the microorganism is a bacterial cell.

In some embodiments, the microorganism expresses an SGD and optionally an STR, CPR, CYB5 and GS and further expresses a Geissoschizine oxidase (GO), a Redox1 and a Redox2, which are not natively present in the cell. Geissoschizine oxidase has an EC number EC 1.14.14.—and catalyzes the oxidation of 19E-geissoschizine to produce a short-lived MIA unstable intermediate which can be oxidized either by Redox1 and Redox2 to produce stemmadenine and 16S/R-deshydroxymethylstemmadenine (16S/R-DHS) or by spontaneous conversion to akuammicine. Redox1 has a EC number EC 1.14.14.—and catalyses the first of two oxidation steps that the converts the unstable product resulting from oxidation of 19E-geissoschizine by geissoschizine oxidase (GO) to stemmadenine. Redox2 has an EC number EC 1.7.1.—and catalyses the second of two oxidation steps that the converts the unstable product resulting from oxidation of 19E-geissoschizine by geissoschizine oxidase (GO) to stemmadenine. The microorganism when expressing GO, Redox1 and Redox2 is thus able to convert 19E-geissoschizine to stemmadenine, thus producing 19E-stemmadenine.

In some embodiments, the microorganism expresses an SGD and optionally an STR, CPR, CYB5 and GS and further expresses GO, Redox1 and Redox2.

In preferred embodiments, the GO is the GO native to Catharanthus roseus or a functional variant thereof which retains the ability to catalyze the oxidation of 19E-geissoschizine to produce a short-lived MIA unstable intermediate which can be oxidized either by Redox1 and Redox2 to produce stemmadenine. Thus in some embodiments, the GO is CroGO as set forth in SEQ ID NO: 34 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 34.

The GO may be expressed in the microorganism by introducing a nucleic acid sequence as detailed further below, which encodes a GO. In particular, the nucleic acid sequence is identical to or has at least 90% identity to SEQ ID NO: 11, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 11.

In preferred embodiments, the Redox1 is the Redox1 native to Catharanthus roseus or a functional variant thereof which retains the ability to catalyse the first of two oxidation steps that the converts the unstable product resulting from oxidation of 19E-geissoschizine by geissoschizine oxidase (GO) to stemmadenine. Thus in some embodiments, the Redox1 is CroRedox1 as set forth in SEQ ID NO: 35 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 35.

The Redox1 may be expressed in the microorganism by introducing a nucleic acid sequence as detailed further below, which encodes a Redox1. In particular, the nucleic acid sequence is identical to or has at least 90% identity to SEQ ID NO: 12, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 12.

In preferred embodiments, the Redox2 is the Redox2 native to Catharanthus roseus or a functional variant thereof which retains the ability to catalyse the second of two oxidation steps that the converts the unstable product resulting from oxidation of 19E-geissoschizine by geissoschizine oxidase (GO) to stemmadenine. Thus in some embodiments, the Redox2 is CroRedox2 as set forth in SEQ ID NO: 36 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 36.

The Redox2 may be expressed in the microorganism by introducing a nucleic acid sequence as detailed further below, which encodes a Redox2. In particular, the nucleic acid sequence is identical to or has at least 90% identity to SEQ ID NO: 13, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 13.

The microorganism further expresses an SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Stemmadenine O-Acetyltransferase

The microorganism may be further engineered so that it can produce O-acetylstemmadenine.

In some embodiments, the microorganism expresses an SGD and optionally an STR, CPR, CYB5, GS, GO, Redox1 and Redox2, and further expresses Stemmadenine O-acetyltransferase which is not natively present in the cell. Stemmadenine O-acetyltransferase has an EC number EC 1.7.1.—and catalyzes the acetylation of stemmadenine to O-acetylstemmadenine. The microorganism when expressing SAT is thus able to convert stemmadenine to O-acetylstemmadenine, thus producing O-acetylstemmadenine.

In some embodiments, the microorganism expresses an SGD and optionally an STR, CPR, CYB5, GS GO, Redox1 and Redox2 and further expresses SAT.

In preferred embodiments, the SAT is the SAT native to Catharanthus roseus or a functional variant thereof which retains the ability to convert stemmadenine to O-acetylstemmadenine. Thus in some embodiments, the SAT is CroSAT as set forth in SEQ ID NO: 37 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity identityto SEQ ID NO: 37.

The SAT may be expressed in the microorganism by introducing a nucleic acid sequence as detailed further below, which encodes a SAT. In particular, the nucleic acid sequence is identical to or has at least 90% identity to SEQ ID NO: 14, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 14.

The microorganism further expresses an SGD as described herein, in particular

RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

O-Acetylstemmadenine Oxidase

The microorganism may be further engineered so that it can produce dihydroprecondylocarpine acetate.

In some embodiments, the microorganism expresses an SGD and optionally an STR, CPR, CYB5, GS, GO, Redox1, Redox2 and SAT, and further expresses O-acetylstemmadenine oxidase (PAS) which is not natively present in the cell. O-acetylstemmadenine oxidase has an EC number EC 1.21.3.—and converts O-acetylstemmadenine to precondylocarpine acetate. The microorganism when expressing PAS is thus able to convert O-acetylstemmadenine to precondylocarpine acetate, thus producing precondylocarpine acetate.

In some embodiments, the microorganism expresses an SGD and optionally an STR, CPR, CYB5, GS GO, Redox1, Redox2, and SAT and further expresses PAS.

In preferred embodiments, the PAS is the PAS native to Catharanthus roseus or a functional variant thereof which retains the ability to convert O-acetylstemmadenine to precondylocarpine acetate. Thus in some embodiments, the PAS is CroPAS as set forth in SEQ ID NO: 38 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 38.

The PAS may be expressed in the microorganism by introducing a nucleic acid sequence as detailed further below, which encodes a PAS. In particular, the nucleic acid sequence is identical to or has at least 90% identity to SEQ ID NO: 15, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 15.

The microorganism further expresses an SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Dehydroprecondylocarpine Acetate Synthase

The microorganism may be further engineered so that it can produce dihydroprecondylocarpine acetate.

In some embodiments, the microorganism expresses an SGD and optionally an STR, CPR, CYB5, GS, GO, Redox1, Redox2, SAT and PAS, and further expresses dihydroprecondylocarpine acetate synthase (DPAS) which is not natively present in the cell. Dihydroprecondylocarpine acetate synthase has an EC number EC 1.1.1.—and converts precondylocarpine acetate to dihydroprecondylocarpine acetate. The microorganism when expressing DPAS is thus able to convert precondylocarpine acetate to dihydroprecondylocarpine acetate, thus producing dihydroprecondylocarpine acetate.

In some embodiments, the microorganism expresses an SGD and optionally an STR, CPR, CYB5, GS GO, Redox1, Redox2, SAT and PAS and further expresses DPAS.

In preferred embodiments, the DPAS is the DPAS native to Catharanthus roseus or a functional variant thereof which retains the ability to convert precondylocarpine acetate to dihydroprecondylocarpine acetate. Thus in some embodiments, the DPAS is CroDPAS as set forth in SEQ ID NO: 39 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 39.

The DPAS may be expressed in the microorganism by introducing a nucleic acid sequence as detailed further below, which encodes a DPAS. In particular, the nucleic acid sequence is identical to or has at least 90% identity to SEQ ID NO: 16, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 16.

The microorganism further expresses an SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Tabersonine Synthase

The microorganism may be further engineered so that it can produce tabersonine.

In some embodiments, the microorganism expresses an SGD and optionally an STR, CPR, CYB5, GS, GO, Redox1, Redox2, SAT, PAS and DPAS, and further expresses Tabersonine synthase (TS) which is not natively present in the cell. Tabersonine synthase has an EC number EC 4.-.-.- and converts dihydroprecondylocarpine acetate to tabersonine. The microorganism when expressing TS is thus able to convert dihydroprecondylocarpine acetate to tabersonine, thus producing tabersonine.

In some embodiments, the microorganism expresses an SGD and optionally an STR, CPR, CYB5, GS GO, Redox1, Redox2, SAT, PAS and DPAS, and further expresses TS.

In preferred embodiments, the TS is the TS native to Catharanthus roseus or a functional variant thereof which retains the ability to convert dihydroprecondylocarpine acetate to tabersonine. Thus in some embodiments, the TS is CroTS as set forth in SEQ ID NO: 40 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 40.

The TS may be expressed in the microorganism by introducing a nucleic acid sequence as detailed further below, which encodes a TS. In particular, the nucleic acid sequence is identical to or has at least 90% identity to SEQ ID NO: 17, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 17.

The microorganism further expresses an SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STD as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Catharanthine Synthase

The microorganism may be further engineered so that it can produce catharanthine.

In some embodiments, the microorganism expresses an SGD and optionally an STR, CPR, CYB5, GS, GO, Redox1, Redox2, SAT, PAS and DPAS, and further expresses Catharanthine synthase (CS) which is not natively present in the cell. Catharanthine synthase has an EC number EC 4.-.-.- and converts dihydroprecondylocarpine acetate to catharanthine. The microorganism when expressing CS is thus able to convert dihydroprecondylocarpine acetate to catharanthine, thus producing catharanthine.

In some embodiments, the microorganism expresses an SGD and optionally an STR, CPR, CYB5, GS GO, Redox1, Redox2, SAT, PAS and DPAS, and further expresses CS. Optionally the microorganism also expresses TS.

In preferred embodiments, the CS is the CS native to Catharanthus roseus or a functional variant thereof which retains the ability to convert dihydroprecondylocarpine acetate to catharanthine. Thus in some embodiments, the CS is CroCS as set forth in SEQ ID NO: 41 or a variant thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 41.

The CS may be expressed in the microorganism by introducing a nucleic acid sequence as detailed further below, which encodes a CS. In particular, the nucleic acid sequence is identical to or has at least 90% identity to SEQ ID NO: 18, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 18.

The microorganism further expresses an SGD as described herein, in particular RseSGD as set forth in SEQ ID NO: 24, GseSGD as set forth in SEQ ID NO: 25, SapSGD as set forth in SEQ ID NO: 26, or RveSGD as set forth in SEQ ID NO: 27, or functional variants thereof having at least 90% identity thereto.

The cell may also further express an STR as described herein, in particular CroSTR as set forth in SEQ ID NO: 30, or a functional variant thereof having at least 90% identity thereto. In some embodiments, the microorganism thus also expresses RseSGD as set forth in SEQ ID NO: 24 and CroSTR as set forth in SEQ ID NO: 30; GseSGD as set forth in SEQ ID NO: 25 and CroSTR as set forth in SEQ ID NO: 30; SapSGD as set forth in SEQ ID NO: 26 and CroSTR as set forth in SEQ ID NO: 30; or RveSGD as set forth in SEQ ID NO: 27 and CroSTR as set forth in SEQ ID NO: 30, or functional variants thereof having at least 90% identity thereto.

Methods for producing strictosidine aglycone and monoterpenoid indole alkaloids The microorganisms described herein are useful as platform for producing plant compounds, in particular strictosidine aglycone and monoterpenoid indole alkaloids (MIAs).

Herein is provided a method of producing strictosidine aglycone in a microorganism, said method comprising the steps of:
a) providing a microorganism, said cell expressing:
   a strictosidine-beta-glucosidase (SGD), capable of converting strictosidine to strictosidine aglycone;
b) incubating said microorganism in a medium comprising strictosidine or a substrate which can be converted to strictosidine by said microorganism;
c) optionally, recovering the strictosidine aglycone;
d) optionally, further converting the strictosidine aglycone to monoterpenoid indole alkaloids.

The microorganism may be as described herein above. Thus, the microorganism may be any microorganism.

Thus, in one embodiment, the microorganism is selected from the group consisting of bacteria, archaea, yeasts, fungi, protozoa, algae, and viruses. In another embodiment, the microorganism is selected from the group consisting of bacteria, archaea, yeasts, fungi, protozoa and algae. In another embodiment, the microorganism is selected from the group consisting of bacteria, archaea, yeasts, fungi, and algae. In another embodiment, the microorganism is selected from the group consisting of bacteria, archaea yeasts and fungi. In another embodiment, the microorganism is selected from bacteria, yeasts and fungi. In another embodiment, the microorganism is selected from bacteria or yeasts. In a preferred embodiment, the microorganism is a bacteria or a yeast.

In some embodiments, the microorganism is a bacteria. In one embodiment, the genus of said bacteria is selected from Escherichia, Corynebacterium, Pseudomonas, Bacillus, Lactococcus, Lactobacillus, Halomonas, Bifidobacterium and Enterococcus. In preferred embodiments, the genus of said bacteria is Escherichia. In another embodiment, the microorganism may be selected from the group consisting of Escherichia, Corynebacterium glutamicum, Pseudomonas putida, Bacillus subtilis, Lactococcus bacillus, Halomonas elongate, Bifidobacterium infantis and Enterococcus faecali. In preferred embodiments, the micororganims is an Escherichia.

In some embodiments, the microorganism is a yeast. In some embodiments, the microorganism is a cell from a GRAS (Generally Recognized As Safe) organism or a non-pathogenic organism or strain. In some embodiments, the genus of said yeast is selected from Saccharomyces, Pichia, Yarrowia, Kluyveromyces, Candida, Rhodotorula, Rhodosporidium, Cryptococcus, Trichosporon and Lipomyces. In preferred embodiments, the genus of said yeast is Saccharomyces.

The microorganism may be selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the microorganism is a Saccharomyces cerevisiae cell.

The strictosidine aglycone produced in the cell may in some embodiments of the methods be further converted into monoterpenoid indole alkaloids. The term “further conversion” herein simply means that the produced strictosidine aglycone is transformed or converted into another compound which is a monoterpenoid indole alkaloid. The conversion may happen in vivo, i.e. within the cell, which may be capable of catalysing further conversion of the strictosidine aglycone into other compounds. The methods however may also comprise the steps of recovering the strictosidine aglycone from the microorganism or from the medium by methods known in the art, and thereafter converting the strictosidine aglycone into monoterpenoid indole alkaloids, i.e. the further conversion may be an ex vivo conversion.

Preferably, the microorganism expresses an SGD as described herein; the SGD may be a heterologous SGD or a mosaic SGD as described herein above. In preferred embodiments, the SGD is selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO:

62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) and functional variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity hereto.

The microorganism may be any of the microorganisms described herein. Thus, the microorganism in some embodiments expresses an SGD as described in the section “Strictosidine-O-beta-glucosidase (SGD)” and is capable of converting strictosidine to strictosidine aglycone. In some embodiments the SGD is a heterologous SGD as described in the section “Heterologous SGD or variants thereof”. In some embodiments, the SGD is a mosaic SGD as described in the section “Mosaic SGD or variants thereof”. The mosaic SGD is as described above and comprises an amino acid sequence having the general formula

D₁-D₂-D₃-D₄

wherein D₁ is a first amino acid sequence from a first SGD,

wherein D₂ is a second amino acid sequence from a second SGD,

wherein D₃ is a third amino acid sequence comprising or consisting of amino acids of SEQ ID NO:91 or a variant thereof having at least 90% identity to SEQ ID NO: 91,

wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

The microorganism may also express an STR as described in the section “Strictosidine synthase (STR)” and may thus be capable of synthesising strictosidine from secologanin and tryptamine. Preferably, secologanin and tryptamine are provided to the cell, e.g. in the medium; in such embodiments, the medium need not comprise strictosidine. In other embodiments, particularly where the microorganism cannot synthesise strictosidine, strictosidine is provided to the microorganism as part of the medium.

The microorganism may be further engineered to produce tetrahydroalstonine as described in the section “Tetrahydroalstonine synthases, heteroyohimbine synthase”. For example, the microorganism may express a heterologous THAS and/or a heterologous HYS.

The microorganism may be further engineered to produce a heteroyohimbine, in particular alstonine and serpentine, as described in the section “Sarpargan bridge enzyme (SBE)”. For example, the microorganism may express a heterologous sarpargan bridge enzyme (SBE).

The microorganism may be further engineered to produce tabersonine and/or caranthine as described herein. In particular, the microorganism may be further engineered to synthesise 19E-geissoschizine as described in the section “NADPH-cytochrome P450 reductase, Cytochrome b5 and Geissoschizine synthase”. For example, the microorganism may express a heterologous NADPH-cytochrome P450 reductase (CPR), a heterologous Cytochrome b5 (CYB5) and a heterologous Geissoschizine synthase (GS). The microorganism may be further engineered so that it can synthesise stemmadenine, as described in the section “Geissoschizine oxidase, Redox1 and Redox2”. For example, the microorganism may express a GO, a Redox1 and a Redox2. The microorganism may be further engineered so that it can synthesise O-acetylstemmadenine as described in section “Stemmadenine O-acetyltransferase”. For example, the microorganism may express SAT. The microorganism may be further engineered so that it can synthesise dihydroprecondylocarpine acetate as described in section “O-acetylstemmadenine oxidase”. For example, the microorganism may express a PAS. The microorganism may be further engineered so that it can produce dihydroprecondylocarpine acetate, as described in the section “Dehydroprecondylocarpine acetate synthase”. For example, the microorganism may express a DPAS. The microorganism may be further engineered so that it can produce tabersonine, as described in the section “Tabersonine synthase”. For example, the microorganism expresses TS. The microorganism may be further engineered so that it can produce catharanthine, as described in the section “Catharanthine synthase”. For example, the microorganism may express a CS.

Thus, the microorganism may be as described above, and may produce one or more of:
strictosidine
strictosidine aglycone
tetrahydroalstonine
alstonine
tabersonine
catharanthine

The necessary substrates for each product may be provided to the cell as part of the medium used to grow the cells. Alternatively, the substrates for each of the above products may be synthesised by the cell itself. In all cases, the microorganism is capable of synthesising strictosidine aglycone.

Each of the above products may be recovered from the medium by methods known in the art if desirable. Accordingly, the method may comprise the step of recovering one or more of:
strictosidine
strictosidine aglycone
tetrahydroalstonine
alstonine
tabersonine
catharanthine

In some embodiments, the medium comprises a substrate which is strictosidine. The microorganism can convert said strictosidine to strictosidine aglycone as described in detail herein above.

In some embodiments, the medium comprises strictosidine, at a concentration of at least 0.05 mM, such as at least 0.1 mM, such as at least 0.5 mM, such as at least 1 mM.

In other embodiments, the medium comprises tryptamine and secologanin, preferably at a concentration of at least 0.05 mM, such as at least 0.1 mM, such as at least 0.5 mM, such as at least 1 mM.

The present invention also related to a method of producing indole alkaloids (MIAs) in a microorganism.

Thus, herein is provided a method of producing monoterpenoid indole alkaloids (MIAs) in a microorganism, said method comprising the steps of:
i) providing a microorganism capable of converting strictosidine to tabersonine and/or catharanthine, said cell expressing:
   a strictosidine-beta-glucosidase (SGD);
   a NADPH-cytochrome P450 reductase (CPR);
   a Cytochrome b5 (CYB5);
   a Geissoschizine synthase (GS);
   a Geissoschizine oxidase (GO);
   a Redox1;
   a Redox2;
   a Stemmadenine O-acetyltransferase (SAT);
   a O-acetylstemmadenine oxidase (PAS);
   a Dehydroprecondylocarpine acetate synthase (DPAS);
   a Tabersonine synthase (TS); and/or
   a Catharanthine synthase (CS);
ii) incubating said microorganism in a medium comprising strictosidine or a substrate which can be converted to strictosidine by said microorganism;
iii) optionally, recovering the MIAs;
iv) optionally, processing the MIAs into a pharmaceutical compound,

wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,

and/or;

wherein said SGD is a mosaic SGD, wherein said mosaic SGD comprises an amino acid sequence having the general formula

D₁-D₂-D₃-D₄

wherein D₁ is a first amino acid sequence from a first SGD,

wherein D₂ is a second amino acid sequence from a second SGD,

wherein D₃ is a third amino acid sequence comprising or consisting of amino acids of SEQ ID NO:91 or a variant thereof having at least 90% identity to SEQ ID NO: 91,

wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.

The microorganism may optionally further express a strictosidine synthase (STR).

The microorganism capable of producing monoterpenoid indole alkaloids (MIAs) may be any microorgsnims as described herein under section “Deteiled description”.

Titers

The microorganisms and methods disclosed herein can be used to produce different plant-derived compounds at high titers. Strictosidine aglycone may thus be obtained with a total titer of at least 0.1 λM, such as at least 0.5 μM, such as at least 1 μM, such as at least 2 μM, such as at least 3 μM, such as at least 4 μM, such as at least 5 μM, such as at least 6 μM, such as at least 7 μM L, such as at least 8 μM, such as at least 9 μM, such as at least 10 μM, such as at least 11 μM, such as at least 12 μM, such as at least 13 μM, such as at least 14 μM, such as at least 15 μM, such as at least 20 μM, such as at least 25 μM, such as at least 30 μM, such as at least 35 μM, such as at least 40 μM, such as at least 50 μM, or more, wherein the total titer is the sum of the intracellular strictosidine aglycone titer and the extracellular strictosidine aglycone. Indeed, the produced strictosidine aglycone may be secreted from the cell—extracellular strictosidine aglycone—or it may be retained in the cell—intracellular strictosidine aglycone.

The microorganism may be capable of producing extracellular strictosidine aglycone with a titer of at least 0.1 μM, such as at least 0.5 μM, such as at least 1 μM, such as at least 2 μM, such as at least 3 μM, such as at least 4 μM, such as at least 5 μM, such as at least 6 μM, such as at least 7 μM L, such as at least 8 μM, such as at least 9 μM, such as at least 10 μM, such as at least 11 μM, such as at least 12 μM, such as at least 13 μM, such as at least 14 μM, such as at least 15 μM, such as at least 20 μM, such as at least 25 μM, such as at least 30 μM, such as at least 35 μM, such as at least 40 μM, such as at least 50 μM, or more.

The microorganism may be capable of producing intracellular strictosidine aglycone with a titer of at least 0.1 μM, such as at least 0.5 μM, such as at least 1 μM, such as at least 2 μM, such as at least 3 μM, such as at least 4 μM, such as at least 5 μM, such as at least 6 μM, such as at least 7 μM L, such as at least 8 μM, such as at least 9 μM, such as at least 10 μM, such as at least 11 μM, such as at least 12 μM, such as at least 13 μM, such as at least 14 μM, such as at least 15 μM, such as at least 20 μM, such as at least 25 μM, such as at least 30 μM, such as at least 35 μM, such as at least 40 μM, such as at least 50 μM, or more.

Methods for determining the strictosidine aglycone titer are known in the art. For example, the cells can be lysed and the titers determined by Orbitrap Fusion Tribid MS (see example 5) to determine the intracellular or secreted strictosidine aglycone titers. The titers can also be determined by Orbitrap Fusion Tribid MS in supernatant fractions from which the cells have been removed.

The microorganism may be capable of producing tetrahydroalstonine with a titre of at least 1 μM, such as at least 2 μM, such as at least 4 μM, such as at least 6 μM, such as at least 8 μM such as at least 10 μM or more.

The microorganism may be capable of producing alstonine with a titre of at least 0.1 μM, such as at least 0.5 μM, such as at least 1 μM, such as at least 2 μM, such as at least 3 μM, such as at least 4 μM, such as at least 5 μM, such as at least 6 μM, such as at least 7 μM L, such as at least 8 μM, such as at least 9 μM, such as at least 10 μM, such as at least 11 μM, such as at least 12 μM, such as at least 13 μM, such as at least 14 μM, such as at least 15 μM, such as at least 20 μM or more.

The microorganism may be capable of producing tabersonine with a titre of at least 0.01 μM, such as at least 0.02 μM, such as at least 0.5 μM, such as at least 1 μM, such as at least 2 μM, such as at least 3 μM, such as at least 4 μM, such as at least 5 μM, such as at least 6 μM, such as at least 7 μM L, such as at least 8 μM, such as at least 9 μM, such as at least 10 μM, such as at least 11 μM, such as at least 12 μM, such as at least 13 μM, such as at least 14 μM, such as at least 15 μM, such as at least 20 μM or more.

The microorganism may be capable of producing catharanthine with a titre of at least 0.01 μM, such as at least 0.02 μM, such as at least 0.5 μM, such as at least 1 μM, such as at least 2 μM, such as at least 3 μM, such as at least 4 μM, such as at least 5 μM, such as at least 6 μM, such as at least 7 μM L, such as at least 8 μM, such as at least 9 μM, such as at least 10 μM, such as at least 11 μM, such as at least 12 μM, such as at least 13 μM, such as at least 14 μM, such as at least 15 μM, such as at least 20 μM or more.

Nucleic Acids, Vectors and Host Cells

Also disclosed herein are useful nucleic acid constructs for constructing a microorganism as described above, or useful in general in the methods described herein. Such nucleic acid constructs encode the heterologous enzymes useful for constructing the microorganisms of the invention.

It will be understood that the term “nucleic acid constructs” may refer to one nucleic acid molecule, or to a plurality of nucleic acid molecules, comprising the relevant nucleic acid sequences. The nucleic acid construct may thus be one nucleic acid molecule, which may encode several enzymes, or it may be several nucleic acid molecules, each comprising one sequence encoding an enzyme. The relevant nucleic acid sequences may thus be comprised on one vector, or on several vectors. They may also be integrated in the genome, on one chromosome or even together in one location, or they may be integrated on different chromosomes. It is also possible to have some sequences on one or more vectors, and some integrated in the genome.

Also provided herein are nucleic acid constructs comprising a nucleic acid sequence identical to or having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 1, SEQ ID NO: 2, SEQ ID NO: 3 or SEQ ID NO: 4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:100, SEQ ID NO:101, SEQ ID NO:102, SEQ ID NO:103, SEQ ID NO:104, SEQ ID NO:105, SEQ ID NO:106 or SEQ ID NO:107. Thus, the microorganism of the invention or the microorganism used in the methods of the invention preferably comprises at least a nucleic acid sequence identical to or having at least 90% identity to SEQ ID NO: 1, SEQ ID NO: 2, SEQ ID NO: 3 or SEQ ID NO: 4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:100, SEQ ID NO:101, SEQ ID NO:102, SEQ ID NO:103, SEQ ID NO:104, SEQ ID NO:105, SEQ ID NO:106 or SEQ ID NO:107. Preferably the nucleic acid is identical to or has at least 90% identity to SEQ ID NO: 1.

As is known in the art, in the event that the first domain of XxxSGD used in the mosaic SGD is not a methionine, the skilled person will readily be able to introduce a start codon in the nucleic acid sequence encoding the mosaic SGD in order to ensure proper translation of the mosaic SGD. The skilled person will also know how to introduce short nucleic acid sequences corresponding to linkers separating the different domains in the mosaic SGD.

The nucleic acid construct may further comprise a nucleic acid sequence identical to or having at 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 7.

The nucleic acid construct may further comprise a sequence identical to or having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 5 and/or SEQ ID NO: 23.

The nucleic acid construct may further comprise a nucleic acid sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 6.

The nucleic acid construct may further comprise a nucleic acid sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and/or SEQ ID NO: 18.

All nucleic acid sequences may have been codon-optimised for expression in the microorganism, as is known in the art.

It may be of interest to take advantage of inducible promoters. Thus in some embodiments, the nucleic acid constructs comprises one or more of the above nucleic acid sequences under the control of an inducible promoter. This allows more control of when the enzyme encoded by the sequence is actually expressed, and can be advantageous for example if production of one of the plant compounds negatively affects cell growth. The skilled person will have no difficulty in identifying suitable inducible promoters.

In some embodiments, the nucleic acid construct is one or more vectors, for examples an integrative or a replicative vector. Suitable vectors are known in the art and readily available to the skilled person.

Also provided herein is a vector comprising one of more of the nucleic acid sequences above, in particular SEQ ID NO: 1 or a sequence having at least 90% identity thereto. The vector may further comprise any of SEQ ID NO: 7, SEQ ID NO: 5, SEQ ID NO: 23, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and/or SEQ ID NO: 18 or a sequence having at least 90% identity thereto.

Also provided herein is a host cell comprising one or more nucleic acid sequence or vector as defined herein above, in particular SEQ ID NO: 1 or a sequence having at least 90% identity thereto, or a vector comprising SEQ ID NO: 1 or a sequence having at least 90% identity thereto, and one or more of SEQ ID NO: 7, SEQ ID NO: 5, SEQ ID NO: 23, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ IDNO: 17 and/or SEQ ID NO: 18 or a sequence having at least 90% identity thereto.

The host cell may be any host cell, such as a primary cell or a cell from a cell line. In preferred embodiments, the host cell is from a mammalian or human cell line. The host cell may be a prokaryote or a eukaryote. In a preferred embodiment, the cell is a eukaryote.

A host cell according to the present invention may be comprised within a host organism, such as an animal.

Also provided herein is the use of the nucleic acid constructs, the microorganisms, the vectors or the host cells described herein for producing strictosidine aglycone and/or tetrahydroalstonine, alstonine, tabersonine and/or catharanthine in a microorganism. In some embodiments, the nucleic acid constructs, the microorganisms, the vectors or the host cells described herein are used in a method for producing strictosidine aglycone and/or tetrahydroalstonine, alstonine, tabersonine and/or catharanthine in a microorganism as described herein.

Pharmaceutical Compounds

The plant compounds obtainable by the present methods may be useful for manufacturing pharmaceutical compounds. Thus, the methods may further comprise a step of producing a pharmaceutical compound from any of the compounds, in particular monoterpenoid indole alkaloids, produced by the microorganism of the present invention.

Thus is also provided a method of treating a disorder such as a cancer, arrhythmia, malaria, psychotic diseases, hypertension, depression, Alzheimer's disease, addiction and/or neuronal diseases, comprising administration of a therapeutic sufficient amount of an MIA or a pharmaceutical compound obtained by the methods described herein.

Sequences

TABLE 1

| Sequence | | |
|---|---|---|
| ID NO: | Description | Details |
| 1 | DNA | Strictosidine-O-beta-D-glucosidase |
| | RseSGD | EC 3.2.1.105 |
| | from Rauvolfia | Hydrolyses strictosidine to strictosidine |
| | serpentina | aglycone |
| 2 | DNA | strictosidine glucosidase |
| | GseSGD | EC 3.2.1.- |
| | from Gelsemium | Putative function: Hydrolyses O- |
| | sempervirens | glycosyl compounds |
| 3 | DNA | 3-alpha-(S)-strictosidine beta- |
| | SapSGD | glucosidase |
| | from Scedosporium | EC 3.2.1.105 |
| | apiospermum | Putative function: Hydrolyses |
| | | strictosidine to strictosidine aglycone |
| 4 | DNA | Strictosidine-beta-D-glucosidase |
| | RveSGD | EC 3.2.1.105 |
| | from Rauvolfia | Putative function: Hydrolyses |
| | verticillata | strictosidine to strictosidine aglycone |
| 5 | DNA | Tetrahydroalstonine synthase |
| | CroTHAS | EC.1.-.-.- |
| | from Chatharanthus | Converts strictosidine aglycone to |
| | roseus | tetrahydroalstonine |
| 6 | DNA | Sarpagan bridge enzyme (CYP71AY5) |
| | GseSBE | EC 1.14.14.- |
| | from Gelsemium | Converts by aromatization the |
| | sempervirens | tetrahydroalstonine and ajmalicine to |
| | | the corresponding alstonine and |
| | | serpentine, respectively or converts by |
| | | cyclization the strictosidine-derived |
| | | geissoschizine to the sarpagan alkaloid |
| | | polyneuridine aldehyde |
| 7 | DNA | Strictosidine synthase |
| | CroSTR from | EC 4.3.3.2 |
| | Catharanthus roseus | Converts secologanin and tryptamine to |
| | | strictosidine by stereospecific |
| | | condensation. |
| 8 | DNA | NADPH-cytochrome P450 reductase |
| | CroCPR from | EC 1.6.2.4 |
| | Catharanthus roseus | This enzyme is required for electron |
| | | transfer from NADP to cytochrome |
| | | P450 |
| 9 | DNA | Cytochrome b5 |
| | CroCYB5 from | EC 1.6.2.2 |
| | Catharanthus roseus | Membrane bound hemoprotein which |
| | | function as an electron carrier |
| 10 | DNA | Geissoschizine synthase (CrADH14) |
| | CroGS from Catharanthus | EC 1.3.1.36 |
| | roseus | Catalyzes the reduction of strictosidine |
| | | aglycone to 19E-geissoschizine |
| 11 | DNA | Geissoschizine oxidase (CYP71AY2) |
| | CroGO from Catharanthus | EC 1.14.14.- |
| | roseus | Catalyzes the oxidation of 19E- |
| | | geissoschizine to produce a short-lived |
| | | MIA unstable intermediate which can |
| | | be oxidized either by Redox1 and |
| | | Redox2 to produce stemmadenine and |
| | | 16S/R- |
| | | deshydroxymethylstemmadenine |
| | | (16S/R-DHS) or by spontaneous |
| | | conversion to akuammicine |
| 12 | DNA | Redox 1 |
| | CroRedox1 from | EC 1.14.14.- |
| | Catharanthus roseus | Catalyzes the first of two oxidation |
| | | steps that the converts the unstable |
| | | product resulting from oxidation of 19E- |
| | | geissoschizine by geissoschizine |
| | | oxidase (GO) to stemmadenine |
| | | biosynthesis |
| 13 | DNA | Redox 2 |
| | CroRedox2 from | EC 1.7.1.- |
| | Catharanthus roseus | Catalyzes the second of two oxidation |
| | | steps that the converts the unstable |
| | | product resulting from oxidation of 19E- |
| | | geissoschizine by geissoschizine |
| | | oxidase (GO) to stemmadenine |
| | | biosynthesis |
| 14 | DNA | Stemmadenine O-acetyltransferase |
| | CroSAT from | EC 1.7.1.- |
| | Catharanthus roseus | Catalyzes the acetylation of |
| | | stemmadenine to O-acetylstemmadenine |
| 15 | DNA | O-acetylstemmadenine oxidase |
| | CroPAS from | (precondylocarpine acetate synthase) |
| | Catharanthus roseus | EC 1.21.3.- |
| | | Converts O-acetylstemmadenine to |
| | | dihydroprecondylocarpine acetate |
| 16 | DNA | Dehydroprecondylocarpine acetate |
| | CroDPAS from | synthase |
| | Catharanthus roseus | EC 1.1.1.- |
| | | Converts precondylocarpine acetate to |
| | | dihydroprecondylocarpine acetate |
| 17 | DNA | tabersonine synthase (Hydrolyase 2) |
| | CroTS from Catharanthus | EC 4.-.-.- |
| | roseus | Catalyzes the conversion of |
| | | dihydroprecondylocarpine acetate to |
| | | tabersonine |
| 18 | DNA | Catharanthine synthase (Hydrolase 1) |
| | CroCS from Catharanthus | EC 4.-.-.- |
| | roseus | Catalyzes the conversion of |
| | | dihydroprecondylocarpine acetate to |
| | | catharanthine |
| 19 | DNA | Putative strictosidine beta-D- |
| | UtoSGD from Uncaria | glucosidase |
| | tomentosa | EC 3.2.1.105 |
| | | Putative function: Hydrolyses |
| | | strictosidine to strictosidine aglycone |
| 20 | DNA | Strictosidine-O-beta-D-glucosidase |
| | CroSGD from | EC 3.2.1.105 |
| | Catharanthus roseus | Hydrolyses strictosidine to strictosidine |
| | | aglycone |
| 21 | DNA | Putative strictosidine beta-D- |
| | CacSGD from | glucosidase |
| | Camptotheca acuminata | EC 3.2.1.105 |
| | | Putative function: Hydrolyses |
| | | strictosidine to strictosidine aglycone |
| 22 | DNA | Uncharacterized protein |
| | GsoSGD from Glycine | EC 3.2.-.- |
| | soja | Putative function: Hydrolyses O- |
| | | glycosyl compounds |
| 23 | DNA | Heteroyohimbine synthase |
| | CroHYS | EC.1.-.-.- |
| | | Converts strictosidine aglycone to |
| | | tetrahydroalstonine, ajmalicine, or |
| | | mayumbine |
| 24 | Protein | Strictosidine-O-beta-D-glucosidase |
| | RseSGD | EC 3.2.1.105 |
| | from Rauvolfia | Q8GU20 |
| | serpentina | Hydrolyses strictosidine to strictosidine |
| | | aglycone |
| 25 | Protein | strictosidine glucosidase |
| | GseSGD | EC 3.2.1.- |
| | from Gelsemium | AXK92564.1 |
| | sempervirens | Putative function: Hydrolyses O- |
| | | glycosyl compounds |
| 26 | Protein | 3-alpha-(S)-strictosidine beta- |
| | SapSGD | glucosidase |
| | from Scedosporium | EC 3.2.1.105 |
| | apiospermum | A0A084GBX6 |
| | | Putative function: Hydrolyses |
| | | strictosidine to strictosidine aglycone |
| 27 | Protein | Strictosidine-beta-D-glucosidase |
| | RveSGD | EC 3.2.1.105 |
| | from Rauvolfia | M9NGS2 |
| | verticillata | Putative function: Hydrolyses |
| | | strictosidine to strictosidine aglycone |
| 28 | Protein | Tetrahydroalstonine synthase |
| | CroTHAS | EC.1.-.-.- |
| | from Chatharanthus | A0A0F6SD02 |
| | roseus | Converts strictosidine aglycone to |
| | | tetrahydroalstonine |
| 29 | Protein | Sarpagan bridge enzyme (CYP71AY5) |
| | GseSBE | EC 1.14.14.- |
| | from Gelsemium | P0DO14 |
| | sempervirens | Converts by aromatization the |
| | | tetrahydroalstonine and ajmalicine to |
| | | the corresponding alstonine and |
| | | serpentine, respectively or converts by |
| | | cyclization the strictosidine-derived |
| | | geissoschizine to the sarpagan alkaloid |
| | | polyneuridine aldehyde |
| 30 | Protein | Strictosidine synthase |
| | CroSTR from | EC 4.3.3.2 |
| | Catharanthus roseus | P18417 |
| | | Converts secologanin and tryptamine to |
| | | strictosidine by stereospecific |
| | | condensation. |
| 31 | Protein | NADPH-cytochrome P450 reductase |
| | CroCPR from | EC 1.6.2.4 |
| | Catharanthus roseus | Q05001 |
| | | This enzyme is required for electron |
| | | transfer from NADP to cytochrome |
| | | P450 |
| 32 | Protein | Cytochrome b5 |
| | CroCYB5 from | EC 1.6.2.2 |
| | Catharanthus roseus | A0A0C5DKP2 |
| | | Membrane bound hemoprotein which |
| | | function as an electron carrier |
| 33 | Protein | Geissoschizine synthase (CrADH14) |
| | CroGS from Catharanthus | EC 1.3.1.36 |
| | roseus | W8JWW7 |
| | | Catalyzes the reduction of strictosidine |
| | | aglycone to 19E-geissoschizine |
| 34 | Protein | Geissoschizine oxidase (CYP71AY2) |
| | CroGO from Catharanthus | EC 1.14.14.- |
| | roseus | I1TEM0 |
| | | Catalyzes the oxidation of 19E- |
| | | geissoschizine to produce a short-lived |
| | | MIA unstable intermediate which can |
| | | be oxidized either by Redox1 and |
| | | Redox2 to produce stemmadenine and |
| | | 16S/R- |
| | | deshydroxymethylstemmadenine |
| | | (16S/R-DHS) or by spontaneous |
| | | conversion to akuammicine |
| 35 | Protein | Redox 1 |
| | CroRedox1 from | EC 1.14.14.- |
| | Catharanthus roseus | A0A2P1GIW4 |
| | | Catalyzes the first of two oxidation |
| | | steps that the converts the unstable |
| | | product resulting from oxidation of 19E- |
| | | geissoschizine by geissoschizine |
| | | oxidase (GO) to stemmadenine |
| | | biosynthesis |
| 36 | Protein | Redox 2 |
| | CroRedox2 from | EC 1.7.1.- |
| | Catharanthus roseus | A0A2P1GIY9 |
| | | Catalyzes the second of two oxidation |
| | | steps that the converts the unstable |
| | | product resulting from oxidation of 19E- |
| | | geissoschizine by geissoschizine |
| | | oxidase (GO) to stemmadenine |
| | | biosynthesis |
| 37 | Protein | Stemmadenine O-acetyltransferase |
| | CroSAT from | EC 1.7.1.- |
| | Catharanthus roseus | A0A2P1GIW7 |
| | | Catalyzes the acetylation of |
| | | stemmadenine to O- |
| | | acetylstemmadenine |
| 38 | Protein | O-acetylstemmadenine oxidase |
| | CroPAS from | (precondylocarpine acetate synthase) |
| | Catharanthus roseus | EC 1.21.3.- |
| | | MH213134.1 |
| | | Converts O-acetylstemmadenine to |
| | | dihydroprecondylocarpine acetate |
| 39 | Protein | Dehydroprecondylocarpine acetate |
| | CroDPAS from | synthase |
| | Catharanthus roseus | EC 1.1.1.- |
| | | A0A1B1FHP3 |
| | | Converts precondylocarpine acetate to |
| | | dihydroprecondylocarpine acetate |
| 40 | Protein | tabersonine synthase (Hydrolyase 2) |
| | CroTS from Catharanthus | EC 4.-.-.- |
| | roseus | A0A2P1GIW3 |
| | | Catalyzes the conversion of |
| | | dihydroprecondylocarpine acetate to |
| | | tabersonine |
| 41 | Protein | Catharanthine synthase (Hydrolase 1) |
| | CroCS from Catharanthus | EC 4.-.-.- |
| | roseus | A0A2P1GIW2 |
| | | Catalyzes the conversion of |
| | | dihydroprecondylocarpine acetate to |
| | | catharanthine |
| 42 | Protein | Putative strictosidine beta-D- |
| | UtoSGD from Uncaria | glucosidase |
| | tomentosa | EC 3.2.1.105 |
| | | I6ZQ42 |
| | | Putative function: Hydrolyses |
| | | strictosidine to strictosidine aglycone |
| 43 | Protein | Strictosidine-O-beta-D-glucosidase |
| | CroSGD from | EC 3.2.1.105 |
| | Catharanthus roseus | B8PRP4 |
| | | Hydrolyses strictosidine to strictosidine |
| | | aglycone |
| 44 | Protein | Putative strictosidine beta-D- |
| | CacSGD from | glucosidase |
| | Camptotheca acuminata | EC 3.2.1.105 |
| | | G8E0P8 |
| | | Putative function: Hydrolyses |
| | | strictosidine to strictosidine aglycone |
| 45 | Protein | Uncharacterized protein |
| | GsoSGD from Glycine | EC 3.2.-.- |
| | soja | A0A0R0H2R3 |
| | | Putative function: Hydrolyses O- |
| | | glycosyl compounds |
| 46 | Protein | Heteroyohimbine synthase |
| | CroHYS from | EC.1.-.-.- |
| | Catharanthus roseus | A0A1B1FHP5 |
| | | Converts strictosidine aglycone to |
| | | tetrahydroalstonine, ajmalicine, or |
| | | mayumbine |
| 47 | Protein VmiSGD1 from | Uncharacterized protein |
| | Vinca minor | EC 3.2.-.- |
| | | Putative function: Hydrolyses O- |
| | | glycosyl compounds |
| 48 | Protein AhuSGD from | Uncharacterized protein |
| | Amsonia hubrichtii | EC 3.2.-.- |
| | | Putative function: Hydrolyses O- |
| | | glycosyl compounds |
| 49 | Protein HimSGD2 from | Uncharacterized protein |
| | Handroanthus | EC 3.2.-.- |
| | impetiginosus | PIN06789.1 |
| | | Putative function: Hydrolyses O- |
| | | glycosyl compounds |
| 50 | Protein SinSGD from | Uncharacterized protein |
| | Sesamum indicum | EC 3.2.-.- |
| | | XP_011094151.1 |
| | | Putative function: Hydrolyses O- |
| | | glycosyl compounds |
| 51 | Protein TelSGD from | Uncharacterized protein |
| | Tabernaemontana | EC 3.2.-.- |
| | elegans | Putative function: Hydrolyses O- |
| | | glycosyl compounds |
| 52 | Protein VunSGD from | Uncharacterized protein |
| | Vigna unguiculata | EC 3.2.-.- |
| | | XP_027910736.1 |
| | | Putative function: Hydrolyses O- |
| | | glycosyl compounds |
| 53 | Protein NsiSGD1 from | Uncharacterized protein |
| | Nyssa sinensis | EC 3.2.-.- |
| | | KAA8549635.1 |
| | | Putative function: Hydrolyses O- |
| | | glycosyl compounds |
| 54 | Protein LprSGD from | Uncharacterized protein |
| | Lomentospora prolificans | EC 3.2.-.- |
| | | PKS11920.1 |
| | | Putative function: Hydrolyses O- |
| | | glycosyl compounds |
| 55 | Protein AchSGD1 from | Uncharacterized protein |
| | Actinidia chinensis var. | EC 3.2.-.- |
| | chinensis | PSS10019.1 |
| | | Putative function: Hydrolyses O- |
| | | glycosyl compounds |
| 56 | Protein HsuSGD from | Uncharacterized protein |
| | Heliocybe sulcata | EC 3.2.-.- |
| | | TFK52902.1 |
| | | Putative function: Hydrolyses O- |
| | | glycosyl compounds |
| 57 | Protein MroSGD from | Uncharacterized protein |
| | Moniliophthora roreri | EC 3.2.-.- |
| | MCA 2997 | ESK96275.1 |
| | | Putative function: Hydrolyses O- |
| | | glycosyl compounds |
| 58 | Protein RseSGD2 from | Raucaffricine-O-beta-D-glucosidase |
| | Rauvolfia serpentina | EC 3.2.1.125 |
| | | AAF03675.1 |
| | | Function: Hydrolyses the MIA |
| | | raucaffricine |
| 59 | Protein PgrSGD from | Uncharacterized protein |
| | Pyricularia grisea | EC 3.2.-.- |
| | | AAX07701.1 |
| | | Putative function: Hydrolyses O- |
| | | glycosyl compounds |
| 60 | Protein OpuSGD from | Uncharacterized protein |
| | Ophiorrhiza pumila | EC 3.2.-.- |
| | | BAP90523.1 |
| | | Putative function: Hydrolyses O- |
| | | glycosyl compounds |
| 61 | Protein HpiSGD from | Uncharacterized protein |
| | Hydnomerulius pinastri | EC 3.2.-.- |
| | MD-312 | KIJ63193.1 |
| | | Putative function: Hydrolyses O- |
| | | glycosyl compounds |
| 62 | Protein HanSGD1 from | Uncharacterized protein |
| | Helianthus annuus | EC 3.2.-.- |
| | | XP_022015317.1 |
| | | Putative function: Hydrolyses O- |
| | | glycosyl compounds |
| 63 | Protein AchSGD2 from | Uncharacterized protein |
| | Actinidia chinensis var. | EC 3.2.-.- |
| | chinensis | PSR88404.1 |
| | | Putative function: Hydrolyses O- |
| | | glycosyl compounds |
| 64 | Protein HimSGD1 from | Uncharacterized protein |
| | Handroanthus | EC 3.2.-.- |
| | impetiginosus | PIN07435.1 |
| | | Putative function: Hydrolyses O- |
| | | glycosyl compounds |
| 65 | Protein IpeSGD from | beta-glucosidase |
| | Carapichea ipecacuanha | EC 3.2.1.21 |
| | | BAH02544.1 |
| | | function: hydrolyses glucosidic Ipecac |
| | | alkaloids |
| 66 | Protein LsaSGD1 from | Uncharacterized protein |
| | Lactuca sativa | EC 3.2.-.- |
| | | XP_023770227.1 |
| | | Putative function: Hydrolyses O- |
| | | glycosyl compounds |
| 67 | Protein CarSGD from | Uncharacterized protein |
| | Coffea arabica | EC 3.2.-.- |
| | | XP_027073002.1 |
| | | Putative function: Hydrolyses O- |
| | | glycosyl compounds |
| 68 | DNA VmiSGD1 from Vinca | Uncharacterized protein |
| | minor | EC 3.2.-.- |
| | | Putative function: Hydrolyses O- |
| | | glycosyl compounds |
| 69 | DNA AhuSGD from | Uncharacterized protein |
| | Amsonia hubrichtii | EC 3.2.-.- |
| | | Putative function: Hydrolyses O- |
| | | glycosyl compounds |
| 70 | DNA HimSGD2 from | Uncharacterized protein |
| | Handroanthus | EC 3.2.-.- |
| | impetiginosus | Putative function: Hydrolyses O- |
| | | glycosyl compounds |
| 71 | DNA SinSGD from | Uncharacterized protein |
| | Sesamum indicum | EC 3.2.-.- |
| | | Putative function: Hydrolyses O- |
| | | glycosyl compounds |
| 72 | DNA TelSGD from | Uncharacterized protein |
| | Tabernaemontana | EC 3.2.-.- |
| | elegans | Putative function: Hydrolyses O- |
| | | glycosyl compounds |
| 73 | DNA VunSGD from Vigna | Uncharacterized protein |
| | unguiculata | EC 3.2.-.- |
| | | Putative function: Hydrolyses O- |
| | | glycosyl compounds |
| 74 | DNA NsiSGD1 from Nyssa | Uncharacterized protein |
| | sinensis | EC 3.2.-.- |
| | | Putative function: Hydrolyses O- |
| | | glycosyl compounds |
| 75 | DNA LprSGD from | Uncharacterized protein |
| | Lomentospora prolificans | EC 3.2.-.- |
| | | Putative function: Hydrolyses O- |
| | | glycosyl compounds |
| 76 | DNA AchSGD1 from | Uncharacterized protein |
| | Actinidia chinensis var. | EC 3.2.-.- |
| | chinensis | Putative function: Hydrolyses O- |
| | | glycosyl compounds |
| 77 | DNA HsuSGD from | Uncharacterized protein |
| | Heliocybe sulcata | EC 3.2.-.- |
| | | Putative function: Hydrolyses O- |
| | | glycosyl compounds |
| 78 | DNA MroSGD from | Uncharacterized protein |
| | Moniliophthora roreri | EC 3.2.-.- |
| | MCA 2997 | Putative function: Hydrolyses O- |
| | | glycosyl compounds |
| 79 | DNA RseSGD2 from | Raucaffricine-O-beta-D-glucosidase |
| | Rauvolfia serpentina | EC 3.2.1.125 |
| | | Function: Hydrolyses the MIA |
| | | raucaffricine |
| 80 | DNA PgrSGD from | Uncharacterized protein |
| | Pyricularia grisea | EC 3.2.-.- |
| | | Putative function: Hydrolyses O- |
| | | glycosyl compounds |
| 81 | DNA OpuSGD from | Uncharacterized protein |
| | Ophiorrhiza pumila | EC 3.2.-.- |
| | | Putative function: Hydrolyses O- |
| | | glycosyl compounds |
| 82 | DNA HpiSGD from | Uncharacterized protein |
| | Hydnomerulius pinastri | EC 3.2.-.- |
| | MD-312 | Putative function: Hydrolyses O- |
| | | glycosyl compounds |
| 83 | DNA HanSGD1 from | Uncharacterized protein |
| | Helianthus annuus | EC 3.2.-.- |
| | | Putative function: Hydrolyses O- |
| | | glycosyl compounds |
| 84 | DNA AchSGD2 from | Uncharacterized protein |
| | Actinidia chinensis var. | EC 3.2.-.- |
| | chinensis | Putative function: Hydrolyses O- |
| | | glycosyl compounds |
| 85 | DNA HimSGD1 from | Uncharacterized protein |
| | Handroanthus | EC 3.2.-.- |
| | impetiginosus | Putative function: Hydrolyses O- |
| | | glycosyl compounds |
| 86 | DNA IpeSGD from | Beta-glucosidase |
| | Carapichea ipecacuanha | EC 3.2.1.21 |
| | | Function: hydrolyses glucosidic Ipecac |
| | | alkaloids |
| 87 | DNA LsaSGD1 from | Uncharacterized protein |
| | Lactuca sativa | EC 3.2.-.- |
| | | Putative function: Hydrolyses O- |
| | | glycosyl compounds |
| 88 | DNA CarSGD from Coffea | Uncharacterized protein |
| | arabica | EC 3.2.-.- |
| | | Putative function: Hydrolyses O- |
| | | glycosyl compounds |
| 89 | Domain 1 of RseSGD from | M1-R115 |
| | Rauvolfia serpentina | MDNTQAEPLVVAIVPKPNASTEHTNS |
| | | HLIPVTRSKIVVHRRDFPQDFIFGAGG |
| | | SAYQCEGAYNEGNRGPSIWDTFTQR |
| | | SPAKISDGSNGNQAINCYHMYKEDIKI |
| | | MKQTGLESYR |
| 90 | Domain 2 of RseSGD from | F116-G266 |
| | Rauvolfia serpentina | FSISWSRVLPGGRLAAGVNKDGVKFY |
| | | HDFIDELLANGIKPSVTLFHWDLPQAL |
| | | EDEYGGFLSHRIVDDFCEYAEFCFWE |
| | | FGDKIKYWTTFNEPHTFAVNGYALGE |
| | | FAPGRGGKGDEGDPAIEPYVVTHNIL |
| | | LAHKAAVEEYRNKFQKCQEG |
| 91 | Domain 3 of RseSGD from | E267-G456 |
| | Rauvolfia serpentina | IGIVLNSMWMEPLSDVQADIDAQKRA |
| | | LDFMLGWFLEPLTTGDYPKSMRELVK |
| | | GRLPKFSADDSEKLKGCYDFIGMNYY |
| | | TATYVTNAVKSNSEKLSYETDDQVTK |
| | | TFERNQKPIGHALYGGWQHVVPWGL |
| | | YKLLVYTKETYHVPVLYVTESGMVEE |
| | | NKTKILLSEARRDAERTDYHQKHLAS |
| | | VRDAIDDG |
| 92 | Domain 4 of RseSGD from | V457-T532 |
| | Rauvolfia serpentina | VNVKGYFVWSFFDNFEWNLGYICRY |
| | | GIIHVDYKSFERYPKESAIWYKNFIAG |
| | | KSTTSPAKRRREEAQVELVKRQKT |
| 93 | Protein sequence of | Mosaic SGD |
| | CCRR | |
| 94 | Protein sequence of | Mosaic SGD |
| | CRRR | |
| 95 | Protein sequence of | Mosaic SGD |
| | RCRR | |
| 96 | Protein sequence of | Mosaic SGD |
| | RRRC | |
| 97 | Protein sequence of | Mosaic SGD |
| | RCRC | |
| 98 | Protein sequence of | Mosaic SGD |
| | CCRC | |
| 99 | Protein sequence of | Mosaic SGD |
| | VVRR | |
| 100 | DNA of CCRR | Mosaic SGD |
| 101 | DNA of CRRR | Mosaic SGD |
| 102 | DNA of RCRR | Mosaic SGD |
| 103 | DNA of CRRC | Mosaic SGD |
| 104 | DNA of RRRC | Mosaic SGD |
| 105 | DNA of RCRC | Mosaic SGD |
| 106 | DNA of CCRC | Mosaic SGD |
| 107 | DNA of VVRR | Mosaic SGD |
| 108 | Protein sequence of | Mosaic SGD |
| | CRRC | |

### EXAMPLES

Strains

Different strains were developed to validate the functionalization of RseSGD in the production of strictosidine aglycone and selected MIAs.

TABLE 2

| Strain | Genotype | Substrate → Product |
|---|---|---|
| MIA-BJ | Cas9 @ XII-1, atf1Δ oye2Δ, oye3Δ ari1Δ | Secologanin + tryptamine |
| | adh6Δ, [CroG8H-CroCYB5] @X-3, | → strictosidine |
| | [CroCPR-Cro8HGO] @XI-3, [CroIS-CroIO] | OR |
| | @XII-2, [CroSTR-CroSLS] @X-4, | Geraniol + tryptamine → |
| | [Cro7DLGT-Cro7DLH] @XI-1, [CroLAMT- | strictosidine |
| | CroADH2] @XII-4 | |
| MIA-CA-1 | Cas9 @ XII-1, atf1Δ oye2Δ, oye3Δ ari1Δ | Secologanin + tryptamine |
| | adh6Δ, [CroG8H-CroCYB5] @X-3, | → strictosidine* |
| | [CroCPR-Cro8HGO] @XI-3, [CroIS-CroIO] | * or tetrahydroalstonine if |
| | @XII-2, [CroSTR-CroSLS] @X-4, | the candidate SGD does |
| | [Cro7DLGT-Cro7DLH] @XI-1, [CroLAMT- | function |
| | CroADH2] @XII-4 [CroSGD- | |
| | CroHYS]@XII-5 | |
| MIA-CA-2 | Cas9 @ XII-1, atf1Δ oye2Δ, oye3Δ ari1Δ | Secolocanin + tryptamine |
| | adh6Δ, [CroG8H-CroCYB5] @X-3, | → tetrahydroalstonine |
| | [CroCPR-Cro8HGO] @XI-3, [CroIS-CroIO] | |
| | @XII-2, [CroSTR-CroSLS] @X-4, | |
| | [Cro7DLGT-Cro7DLH] @XI-1, [CroLAMT- | |
| | CroADH2] @XII-4 [RseSGD- | |
| | CroHYS]@XII-5 | |
| MIA-CA-3 | Cas9 @ XII-1, atf1Δ oye2Δ, oye3Δ ari1Δ | Secolocanin + tryptamine |
| | adh6Δ, [CroG8H-CroCYB5] @X-3, | → tetrahydroalstonine |
| | [CroCPR-Cro8HGO] @XI-3, [CroIS-CroIO] | |
| | @XII-2, [CroSTR-CroSLS] @X-4, | |
| | [Cro7DLGT-Cro7DLH] @XI-1, [CroLAMT- | |
| | CroADH2] @XII-4 [RveSGD- | |
| | CroHYS]@XII-5 | |
| MIA-CA-4 | Cas9 @ XII-1, atf1Δ oye2Δ, oye3Δ ari1Δ | Secolocanin + tryptamine |
| | adh6Δ, [CroG8H-CroCYB5] @X-3, | → tetrahydroalstonine |
| | [CroCPR-Cro8HGO] @XI-3, [CroIS-CroIO] | |
| | @XII-2, [CroSTR-CroSLS] @X-4, | |
| | [Cro7DLGT-Cro7DLH] @XI-1, [CroLAMT- | |
| | CroADH2] @XII-4 [GseSGD- | |
| | CroHYS]@XII-5 | |
| MIA-CA-5 | Cas9 @ XII-1, atf1Δ oye2Δ, oye3Δ ari1Δ | Secolocanin + tryptamine |
| | adh6Δ, [CroG8H-CroCYB5] @X-3, | → strictosidine* |
| | [CroCPR-Cro8HGO] @XI-3, [CroIS-CroIO] | * or tetrahydroalstonine if |
| | @XII-2, [CroSTR-CroSLS] @X-4, | the candidate SGD does |
| | [Cro7DLGT-Cro7DLH] @XI-1, [CroLAMT- | function |
| | CroADH2] @XII-4 [CacSGD- | |
| | CroHYS]@XII-5 | |
| MIA-CA-6 | Cas9 @ XII-1, atf1Δ oye2Δ, oye3Δ ari1Δ | Secolocanine + tryptamine |
| | adh6Δ, [CroG8H-CroCYB5] @X-3, | → tetrahydroalstonine |
| | [CroCPR-Cro8HGO] @XI-3, [CroIS-CroIO] | |
| | @XII-2, [CroSTR-CroSLS] @X-4, | |
| | [Cro7DLGT-Cro7DLH] @XI-1, [CroLAMT- | |
| | CroADH2] @XII-4 [SapSGD- | |
| | CroHYS]@XII-5 | |
| MIA-CA-7 | Cas9 @ XII-1, atf1Δ oye2Δ, oye3Δ ari1Δ | Secolocanin + tryptamine |
| | adh6Δ, [CroG8H-CroCYB5] @X-3, | → strictosidine* |
| | [CroCPR-Cro8HGO] @XI-3, [CroIS-CroIO] | * or tetrahydroalstonine if |
| | @XII-2, [CroSTR-CroSLS] @X-4, | the candidate SGD does |
| | [Cro7DLGT-Cro7DLH] @XI-1, [CroLAMT- | function |
| | CroADH2] @XII-4 [UtoSGD- | |
| | CroHYS]@XII-5 | |
| MIA-CA-8 | Cas9 @ XII-1, atf1Δ oye2Δ, oye3Δ ari1Δ | Secolocanin + tryptamine |
| | adh6Δ, [CroG8H-CroCYB5] @X-3, | → strictosidine* |
| | [CroCPR-Cro8HGO] @XI-3, [CroIS-CroIO] | * or tetrahydroalstonine if |
| | @XII-2, [CroSTR-CroSLS] @X-4, | the candidate SGD does |
| | [Cro7DLGT-Cro7DLH] @XI-1, [CroLAMT- | function |
| | CroADH2] @XII-4 [GsoSGD- | |
| | CroHYS]@XII-5 | |
| MIA-BZ-1 | Cas9 @ XII-1, atf1Δ oye2Δ, oye3Δ ari1Δ | Secolocanin + tryptamine |
| | adh6Δ, [CroG8H-CroCYB5] @X-3, | → strictosidine* |
| | [CroCPR-Cro8HGO] @XI-3, [CroIS-CroIO] | * or strictosidine aglycone |
| | @XII-2, [CroSTR-CroSLS] @X-4, | if the candidate SGD does |
| | [Cro7DLGT-Cro7DLH] @XI-1, [CroLAMT- | function |
| | CroADH2] @XII-4 [CroSGD]@XII-5 | |
| MIA-BZ-2 | Cas9 @ XII-1, atf1Δ oye2Δ, oye3Δ ari1Δ | Secolocanin + tryptamine |
| | adh6Δ, [CroG8H-CroCYB5] @X-3, | → strictosidine aglycone |
| | [CroCPR-Cro8HGO] @XI-3, [CroIS-CroIO] | |
| | @XII-2, [CroSTR-CroSLS] @X-4, | |
| | [Cro7DLGT-Cro7DLH] @XI-1, [CroLAMT- | |
| | CroADH2] @XII-4 [RseSGD]@XII-5 | |
| MIA-BZ-3 | Cas9 @ XII-1, atf1Δ oye2Δ, oye3Δ ari1Δ | Secolocanin + tryptamine |
| | adh6Δ, [CroG8H-CroCYB5] @X-3, | → strictosidine* |
| | [CroCPR-Cro8HGO] @XI-3, [CroIS-CroIO] | * or tetrahydroalstonine if |
| | @XII-2, [CroSTR-CroSLS] @X-4, | the candidate SGD does |
| | [Cro7DLGT-Cro7DLH] @XI-1, [CroLAMT- | function |
| | CroADH2] @XII-4 [CroSGD- | |
| | CroTHAS]@XII-5 | |
| MIA-BZ-4 | Cas9 @ XII-1, atf1Δ oye2Δ, oye3Δ ari1Δ | Secolocanin + tryptamine |
| | adh6Δ, [CroG8H-CroCYB5] @X-3, | → tetrahydroalstonine |
| | [CroCPR-Cro8HGO] @XI-3, [CroIS-CroIO] | |
| | @XII-2, [CroSTR-CroSLS] @X-4, | |
| | [Cro7DLGT-Cro7DLH] @XI-1, [CroLAMT- | |
| | CroADH2] @XII-4 [RseSGD- | |
| | CroTHAS]@XII-5 | |
| MIA-DA | Cas9@XII-1, atf1Δ oye2Δ, oye3Δ ari1Δ | No production |
| | adh6Δ, [CroCPR-CroCYB5]@XI-3 | |
| MIA-DC | Cas9@XII-1, atf1Δ oye2Δ, oye3Δ ari1Δ | Secologanin + tryptamine |
| | adh6Δ, [CroCPR-CroCYB5]@XI-3, | → tabersonine + |
| | [CroSTR-CroGS-RseSGD-CroGO- | catharanthine |
| | CroRedoxI -CroRedox2]@XII-5, [CroSAT- | |
| | CroPAS-CroDPAS-CroTS-CroCG]@XI-5 | |
| MIA-DE | Cas9@XII-1, atf1Δ oye2Δ, oye3Δ ari1Δ | tabersonine → Vindoline |
| | adh6Δ, [CroCPR-CroCYB5]@XI-3, | OR |
| | [CroNMT-CroD4H-CroDAT-CroPER- | Tabersonine + |
| | CroT16H1]@X-4, [CroT16H2-Cro16OMT- | catharanthine → |
| | CroT3O-CroT3R]@XII-4 | vinblastine |
| | | OR |
| | | Vindoline + catharanthine |
| | | → vinblastine |
| MIA-FA | Cas9 @ XII-1, atf1Δ oye2Δ, oye3Δ ari1Δ | Secologanin + tryptamine |
| | adh6Δ, [CroG8H-CroCYB5] @X-3, | → strictosidine* |
| | [CroCPR] @XI-3, [CroIO] @XII-2, | OR |
| | [CroSTR-CroSLS] @X-4, [Cro7DLGT- | Geraniol + tryptamine → |
| | Cro7DLH] @XI-1, [CroLAMT-CroADH2] | strictosidine* |
| | @XII-4, [Vmi8HGO-A] @X-2, [NcMLP- | *or tetrahydroalstonine if |
| | NcISY] @XII-5, [CroHYS] @IV-1 | functional SGD is co- |
| | | expressed |
| MIA-FC-1 | Cas9 @ XII-1, atf1Δ oye2Δ, oye3Δ ari1Δ | Secolocanin + tryptamine |
| | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [CroSGD] @IV-2 | |
| MIA-FC-2 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [VmiSGDI] @IV-2 | |
| MIA-FC-3 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [AhuSGD] @IV-2 | |
| MIA-FC-4 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [HimSGD2] @IV-2 | |
| MIA-FC-5 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [SinSGD] @IV-2 | |
| MIA-FC-6 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [TelSGD] @IV-2 | |
| MIA-FC-7 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [VunSGD] @IV-2 | |
| MIA-FC-8 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [NsiSGD1] @IV-2 | |
| MIA-FC-9 | Cas9 @ XII-1, atf1Δ oye2Δ, oye3Δ ari1Δ | Secologanin + tryptamine |
| | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [LprSGD] @IV-2 | |
| MIA-FC- | Cas9 @ XII-1, atf1Δ oye2Δ, oye3Δ ari1Δ | Secologanin + tryptamine |
| 10 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [AchSGD1] @IV-2 | |
| MIA-FC- | Cas9 @ XII-1, atf1Δ oye2Δ, oye3Δ ari1Δ | Secologanin + tryptamine |
| 11 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [HsuSGD] @IV-2 | |
| MIA-FC- | Cas9 @ XII-1, atf1Δ oye2Δ, oye3Δ ari1Δ | Secologanin + tryptamine |
| 12 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [MroSGD] @IV-2 | |
| MIA-FC- | Cas9 @ XII-1, atf1Δ oye2Δ, oye3Δ ari1Δ | Secologanin + tryptamine |
| 13 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [RseSGD2] @IV-2 | |
| MIA-FC- | Cas9 @ XII-1, atf1Δ oye2Δ, oye3Δ ari1Δ | Secologanin + tryptamine |
| 14 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [PgrSGD] @IV-2 | |
| MIA-FC- | Cas9 @ XII-1, atf1Δ oye2Δ, oye3Δ ari1Δ | Secologanin + tryptamine |
| 15 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [OpuSGD] @IV-2 | |
| MIA-FC- | Cas9 @ XII-1, atf1Δ oye2Δ, oye3Δ ari1Δ | Secologanin + tryptamine |
| 16 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [HpiSGD] @IV-2 | |
| MIA-FC- | Cas9 @ XII-1, atf1Δ oye2Δ, oye3Δ ari1Δ | Secologanin + tryptamine |
| 17 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [HanSGD1] @IV-2 | |
| MIA-FC- | Cas9 @ XII-1, atf1Δ oye2Δ, oye3Δ ari1Δ | Secologanin + tryptamine |
| 18 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [AchSGD2] @IV-2 | |
| MIA-FC- | Cas9 @ XII-1, atf1Δ oye2Δ, oye3Δ ari1Δ | Secologanin + tryptamine |
| 19 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [HimSGD1] @IV-2 | |
| MIA-FC- | Cas9 @ XII-1, atf1Δ oye2Δ, oye3Δ ari1Δ | Secologanin + tryptamine |
| 20 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [IpeSGD] @IV-2 | |
| MIA-FC- | Cas9 @ XII-1, atf1Δ oye2Δ, oye3Δ ari1Δ | Secologanin + tryptamine |
| 21 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [LsaSGD1] @IV-2 | |
| MIA-FC- | Cas9 @ XII-1, atf1Δ oye2Δ, oye3Δ ari1Δ | Secologanin + tryptamine |
| 22 | adh6Δ, [CroG8H-CroCYB5] @X-3, | → tetrahydroalstonine |
| | [CroCPR] @XI-3, [CroIO] @XII-2, | |
| | [CroSTR-CroSLS] @X-4, [Cro7DLGT- | |
| | Cro7DLH] @XI-1, [CroLAMT-CroADH2] | |
| | @XII-4, [Vmi8HGO-A] @X-2, [NcMLP- | |
| | NcISY] @XII-5, [CroHYS] @IV-1, | |
| | [CarSGD] @IV-2 | |
| MIA-FC- | Cas9 @ XII-1, atf1Δ oye2Δ, oye3Δ ari1Δ | Secolocanin + tryptamine |
| 23 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [OeuSGD2] @IV-2 | |
| MIA-FC- | Cas9 @ XII-1, atf1Δ oye2Δ, oye3Δ ari1Δ | Secolocanin + tryptamine |
| 24 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [AchSGD3] @IV-2 | |
| MIA-FC- | Cas9 @ XII-1, atf1Δ oye2Δ, oye3Δ ari1Δ | Secolocanin + tryptamine |
| 25 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [CmaSGD] @IV-2 | |
| MIA-FC- | Cas9 @ XII-1, atf1Δ oye2Δ, oye3Δ ari1Δ | Secolocanin + tryptamine |
| 26 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [MmySGD] @IV-2 | |
| MIA-FC- | Cas9 @ XII-1, atf1Δ oye2Δ, oye3Δ ari1Δ | Secolocanin + tryptamine |
| 27 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [VmiSGD3] @IV-2 | |
| MIA-FC- | Cas9 @ XII-1, atf1Δ oye2Δ, oye3Δ ari1Δ | Secolocanin + tryptamine |
| 28 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [IniSGD] @IV-2 | |
| MIA-FC- | Cas9 @ XII-1, atf1Δ oye2Δ, oye3Δ ari1Δ | Secolocanin + tryptamine |
| 29 | adh6Δ, [CroG8H-CroCYB5] @X-3, | → strictosidine* |
| | [CroCPR] @XI-3, [CroIO] @XII-2, | * or tetrahydroalstonine if |
| | [CroSTR-CroSLS] @X-4, [Cro7DLGT- | the candidate SGD does |
| | Cro7DLH] @XI-1, [CroLAMT-CroADH2] | function |
| | @XII-4, [Vmi8HGO-A] @X-2, [NcMLP- | |
| | NcISY] @XII-5, [CroHYS] @IV-1, | |
| | [NsiSGD2] @IV-2 | |

### Example 1

Construction of USER Backbones

All USER vectors were constructed based on pCfB2315 (pRS413-HIS), linearized by restriction enzymes Xhol and Sac! (Thermo-Fisher FastDigest™). All terminators were amplified from CEN.PK113-7D genome using primers flanked with Xhol and Sac! restriction sites. A DNA cassette containing the ccdB counter-selection marker (Steyaert J. et al. 1993) was inserted into all USER vectors to ensure high cloning efficiency.

USER Assembly of Plasmids

All plasmids were constructed using the USER method (Jensen NB et al. 2013). Biobrick for plant genes were amplified from synthetic gBlocks (Integrated DNA Technologies and Twist Biosciences), codon optimized for expression in yeast host. Biobrick for promoters were amplified from yeast CEN.PK113-7D genome.

Construction of Strains

All strains were constructed using the CRISPR-Cas9 method described in Jakoc̆iūnas T. et al. 2015.

### Example 2

Showing that CroSGD does not Function in Yeast

Geerlings et al. (Geerlings, A., 2000 and WO 00/42200) originally isolated a full-length cDNA clone from a Catharanthus roseus cDNA library giving rise to SGD activity in an in vitro assay.

To confirm if CroSGD could be validated and functionalized in yeast, CroSGD was expressed according to Geerlings et al. by using the strong glycolytic and constitutive active promoters TDH3 and TEF1, respectively.

The following yeast strains were produced, containing SGD and tetrahydroalstonine (THA) synthase both from Catharantus roseus, i.e. CroSGD and CroTHAS.

Strain MIA-BJ (EZ-Swap, full CroSTR) expressing:
P1-TDH3-CroSGD_nls-P2_TEF1-CroTHAS_nls
P1-TDH3-CroSGD_cyt-P2_TEF1-CroTHAS_cyt
P2-TEF1-CroSGD-5xGS-CroTHAS_nls
P2-TEF1-CroTHAS-5xGS-CroSGD_nls
P2-TEF1-CroSGD-5xGS-CroTHAS_cyt
P2-TEF1-CroTHAS-5xGS-CroSGD_cyt
P1-TEF1-CroSGD_nls-P2_PGK1-CroTHAS_nls
P1-TEF1-CroSGD_cyt-P2_PGK1-CroTHAS_cyt
P1-TEF1-CroSGD_nls-P2_PGK1-CroTHAS_cyt
P1-TEF1-CroSGD_cyt-P2_PGK1-CroTHAS_nls

The high-resolution analytical results obtained from LC-MS analysis expressing CroSGD alone and in various tagged and CroSGD-fusion versions contradicts the results presented by Geerlings et al. are not valid.

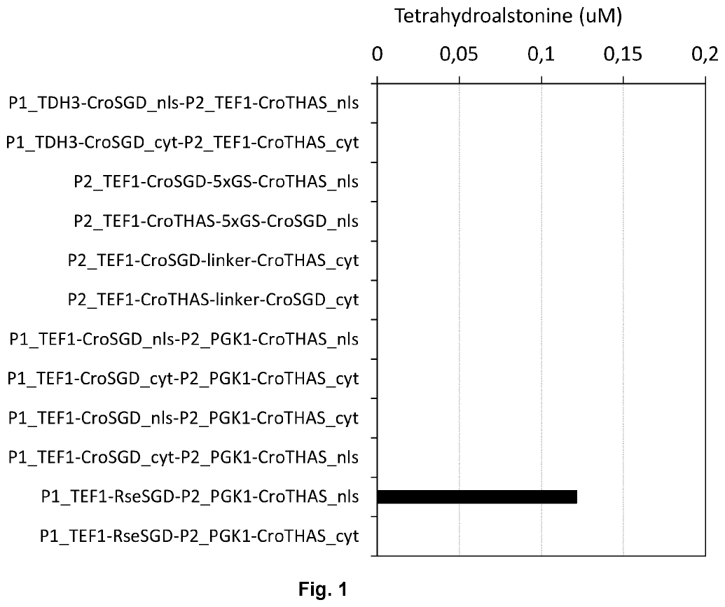

FIG. 1 shows the LC-MS analysis of tetrahydroalstonine (THA). From FIG. 1 it can be seen that none of the strains expressing CroSGD could produce detectable amount of tetrahydroalstonine.

As a positive control, the following strains were created, strain MIA-BJ (EZ-Swap, full CroSTR) expressing:
P1-TEF1-RseSGD-P2_PGK1-CroTHAS_nls
P1-TEF1-RseSGD-P2_PGK1-CroTHAS_cyt

Surprisingly, and in contrast to the strains expressing CroSGD, the yeast stain expressing RseSGD (P1-TEF1-RseSGD-P2_PGK1-CroTHAS_nls) was able to produce tetrahydroalstonine, thus showing that RseSGD is functional in yeast (FIG. 1). Tetrahydroalstonine was detected in both samples from supernatant (filtered medium) and cell pellet.

### Example 3

SGD Homology Search

To further investigate, and ultimately enable, functionalization of the critical SGD node in yeast, a homology-search for SGDs against the NCBI database and using the CroSGD protein sequence as a query was performed. From this search, eight different SGD homologs from Catharanthus roseus (CroSGD), Rauvolfia serpentina (RseSGD), Rauvolfia verticillata (RveSGD), Gelsemium sempervirens (GseSGD), Camptotheca acuminate (CacSGD), Scedosporium apiospermum (SapSGD), Uncaria tomentosa (UtoSGD) and Glycine soja (GsoSGD) were selected.

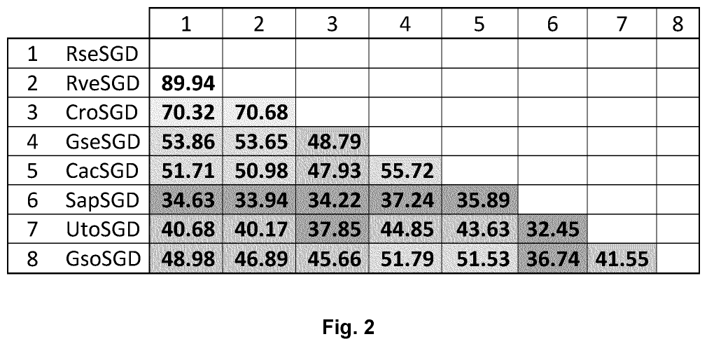

The eight protein sequences were aligned with the t-Coffee web server (FIG. 2).

Among the eight SGDs selected for this test, two (Catharanthus roseus and Rauvolfia serpentina) are known to have SGD activity in vitro, four are putative SGD from MIA producing plants (Rauvolfia verticillata, Gelsemium sempervirens, Camptotheca acuminate and Uncaria tomentosa). Scedosporium apiospermum is a fungus known to produce other alkaloids. Glycine soja, which is unlikely to have SGD activity, was chosen as a negative control. See table 3 below.

TABLE 3

| | | | | MIA production |
|---|---|---|---|---|
| | | | | in the origin |
| Abbreviation | Function | Species | Family | organism |
| RseSGD | In vitro | Rauvolfia serpentina | Apocyanaceae | Yes |
| | verified | | | |
| | SGD | | | |
| RveSGD | Putative | Rauvolfia verticillate | Apocyanaceae | Yes |
| | SGD | | | |
| CroSGD | In vitro | Catharanthus roseus | apocyanaceae | Yes |
| | verified | | | |
| | SGD | | | |
| GseSGD | Putative | Gelsemium | Gelsemiacea | Yes |
| | SGD | sempervirens | | |
| UtoSGD | Putative | Uncaria tomentosa | Rubiaceae | Yes |
| | SGD | | | |
| CacSGD | Putative | Camptotheca | Nyssaseae | Yes |
| | SGD | acuminata | | |
| SapSGD | Putative | Scedosporium | Microascaceae | Yes |
| | SGD | apiospermum | (fungi) | |
| GsoSGD | Putative | Glycine soja | Phaseoleae | No |
| | GH1 beta- | | | |
| | gucosidase | | | |

Each one of the eight SGD together with the CroHYS (capable of converting strictosidine aglycone to tetrahydroalsoinine) gene were integrated into a MIA-BJ strain expressing CroG8H+CroCYB5+CroCPR+Cro8HGO+CrolS+CrolO+CroSTR+CroSLS+Cro7DLGT+Cro7DLH+CroLAMT+CroADH2, resulting in strains MIA-CA-1 to MIA-CA-8

MIA-CA-1: MIA-BJ strain+CroSGD+CroHYS

MIA-CA-2: MIA-BJ strain+RseSGD+CroHYS

MIA-CA-3: MIA-BJ strain+RveSGD+CroHYS

MIA-CA-4: MIA-BJ strain+GseSGD+CroHYS

MIA-CA-5: MIA-BJ strain+CacSGD+CroHYS

MIA-CA-6: MIA-BJ strain+SapSGD+CroHYS

MIA-CA-7: MIA-BJ strain+UtoSGD+CroHYS

MIA-CA-8: MIA-BJ strain+GsoSGD+CroHYS

First, all strains were grown (in triplicates) in 150 uL of YPD for overnight to saturation. Then, 10 ul preculture was transferred into 500 uL of synthetic complete (SC) medium with 2% glucose, supplemented with 0.1 mM of secologanin and 1 mM of tryptamine. After 6 days, 200 uL supernatant was filtered through a 0.2 pm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure secologanin, strictosidine and tetrahydroalstonine concentrations.

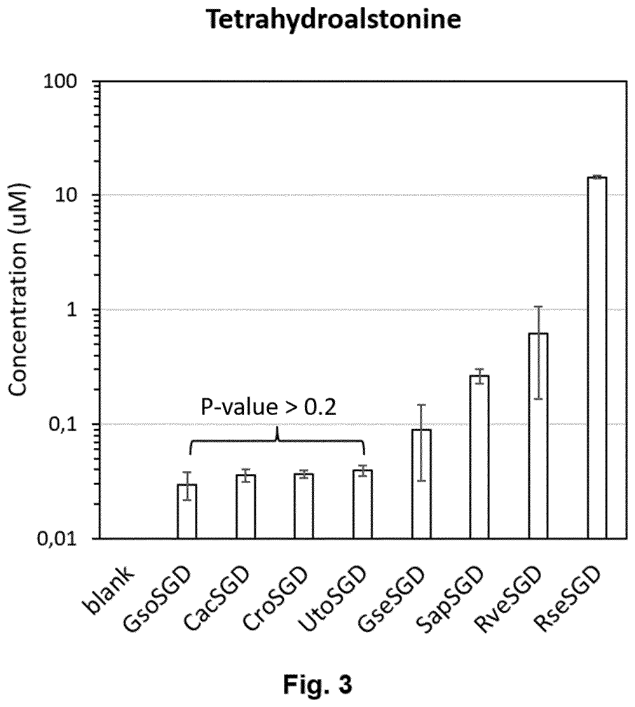

Yeast strains expressing GseSGD, SapSGD, RveSGD and RseSGD were able to produce tetrahydroalstonine (FIG. 3). Whereas, CacSGD, CroSGD and UtoSGD, as well as their control GsSGD were not able to produce tetrahydroalstonine. The p-value represents comparison between the negative control (GsoSGD) and each of CacSGD, CroSGD and UtoSGD.

The yeast strain expressing RseSGD was able to produce at least 10 μM tetrahydroalstonine.

### Example 4

Cellular Localisation and Expression

In order to understand the functional discrepancy between CroSGD and RseSGD in yeast, the two enzymes were GFP-tagged and their subcellular localization was studied. A clear difference in both level of expression and localization was observed for CroSGD and RseSGD.

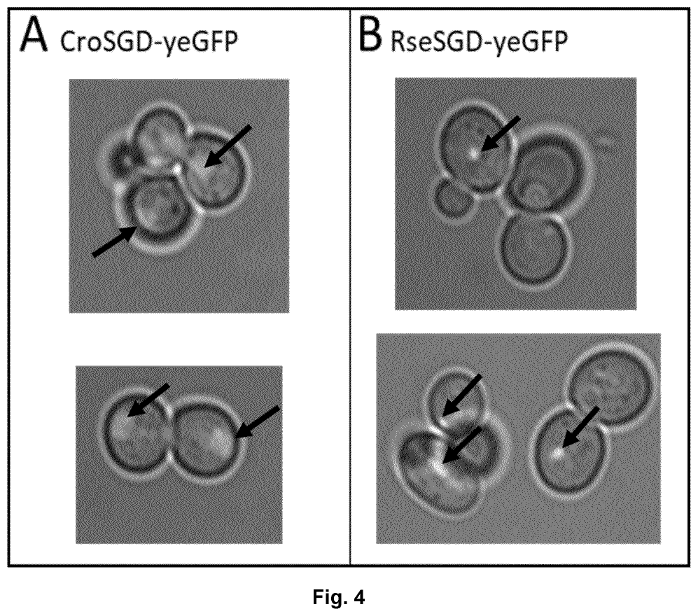

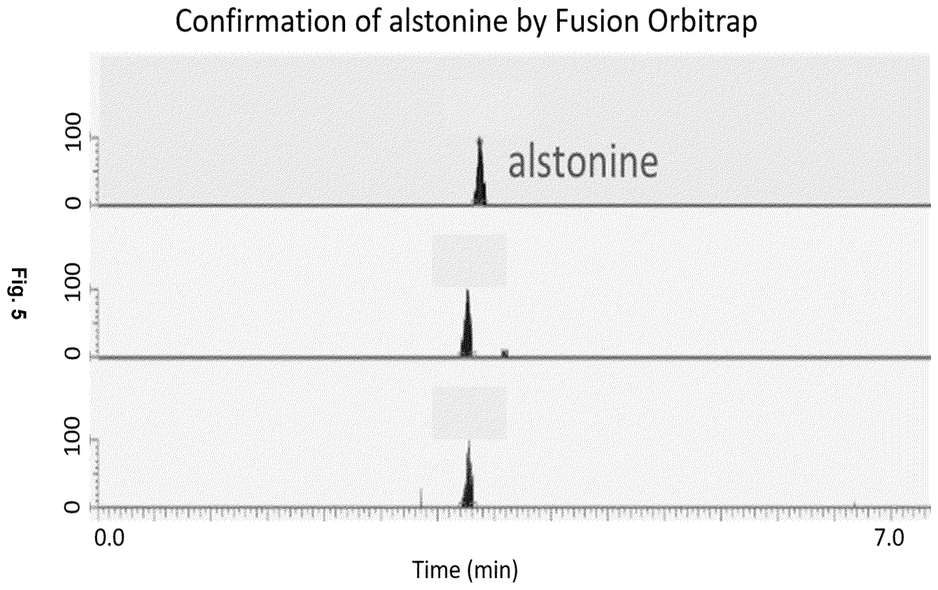

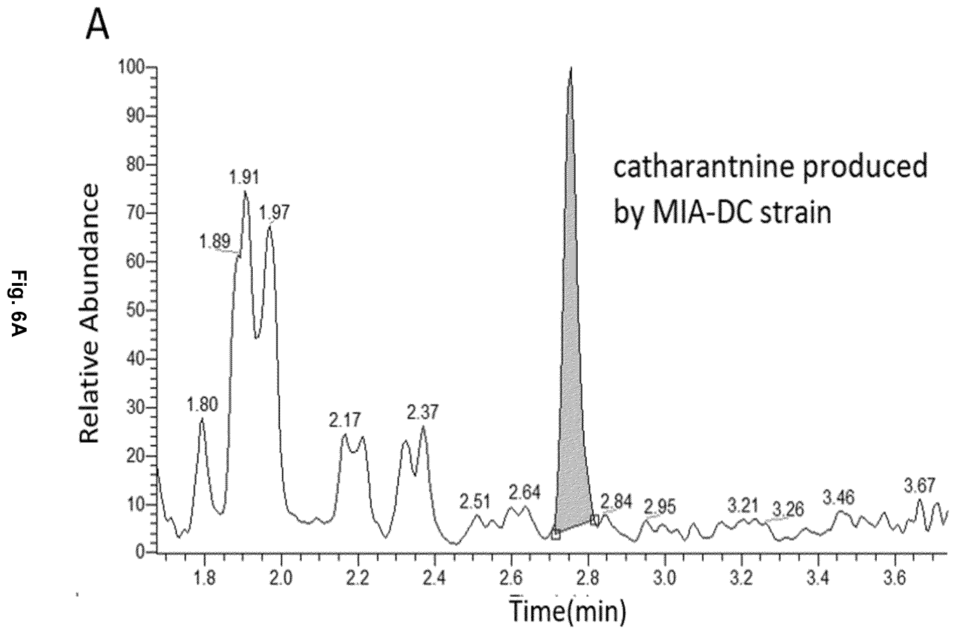

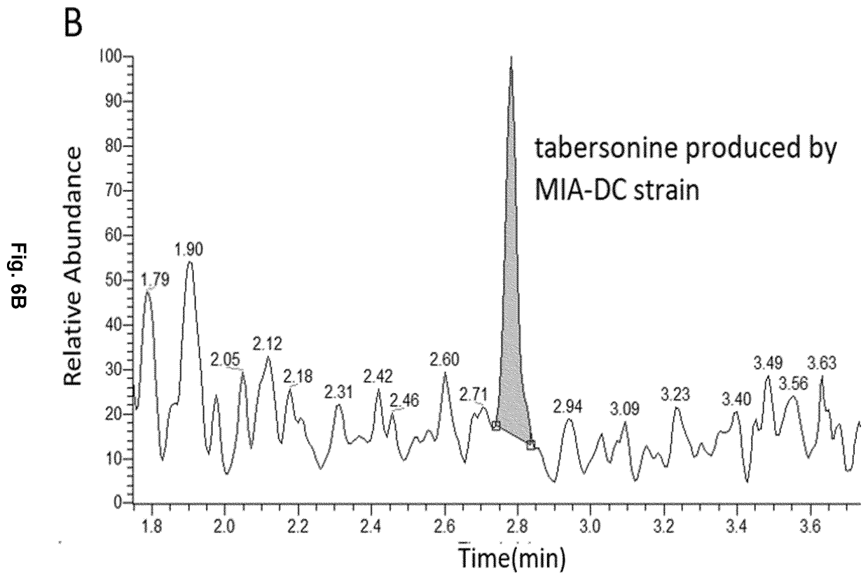

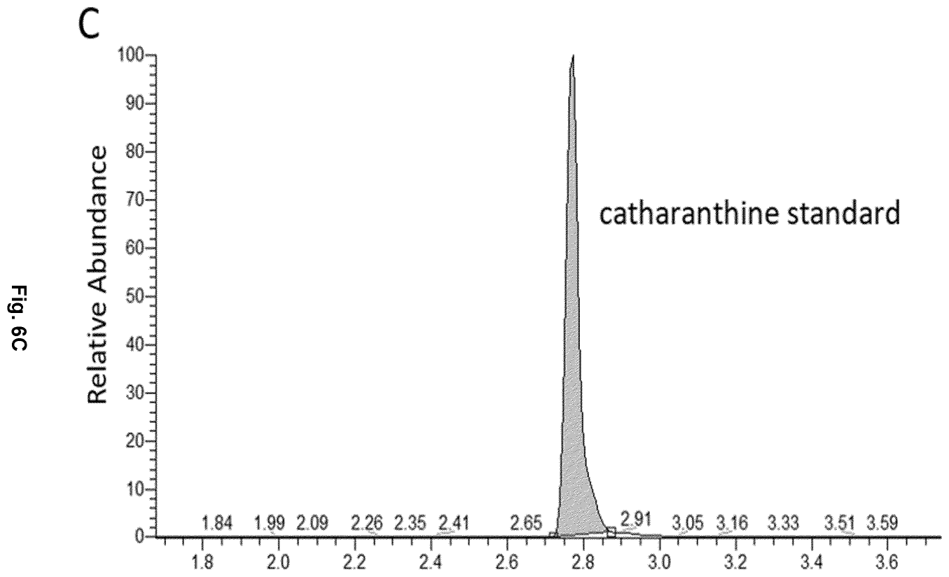

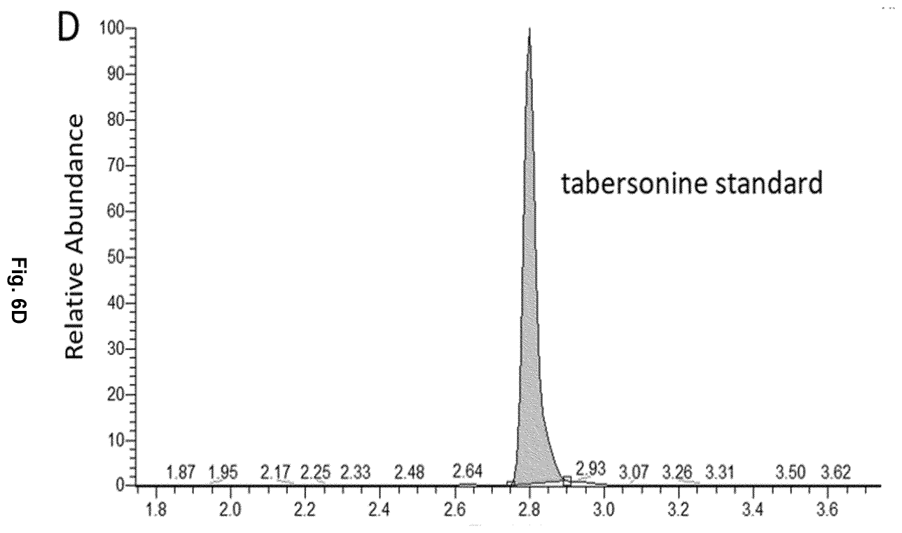

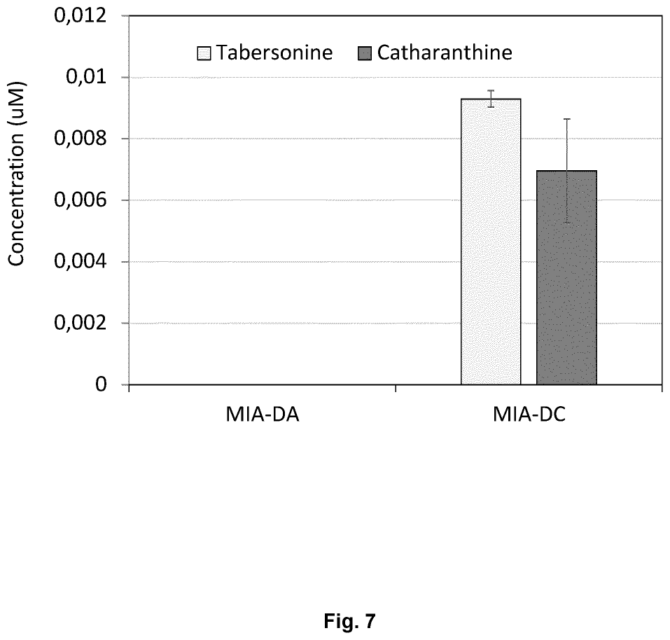

The yeast cells expressing GFP-linker-CroSGD showed weak expression of CroSGD, as well as a nuclear localization of the CroSGD, whereas the yeast cells expressing GFP-linker-RseSGD showed higher RseSGD expression and a supramolecular localization pattern (FIG. 4) resembling CroSGD localization in planta.

### Example 5

Production of Strictosidine Aglycone and Heteroyohimbines

Strictosidine Aglycone and Tetrahydroalstonine

CroSGD or RseSGD alone or in combination with the CroTHAS were inserted into the MIA-BJ strain (CroG8H+CroCYB5+CroCPR+Cro8HGO+CrolS+CrolO+CroSTR+CroSLS+Cro7DLGT+Cro7DLH+CroLAMT+CroADH2), resulting in strains MIA-BZ-1 to MIA-BZ-4:
MIA-BZ-1: MIA-BJ strain+pTEF1->CroSGD-tADH1
MIA-BZ-2: MIA-BJ strain+p TEF1->RseSGD-tADH 1
MIA-BZ-3: MIA-BJ strain+tCYC1-CroTHAS<-pPGK1-pTEF1->CroSGD-tADH1
MIA-BZ-4: MIA-BJ strain+tCYC1-CroTHAS<-pPGK1-pTEF1->RseSGD-tADH1

The yeast strains MIA-BZ-1 to MIA-BZ-4 as well as their control (MIA-BJ strain), were tested in batch fermentation using 96-well deep plate as the following.

First, all strains were grown (in triplicates) in 150 uL of YPD for overnight to saturation. Then, 10 ul preculture was transferred into 500 uL of synthetic complete (SC) medium with 2% glucose, supplemented with 0.1 mM of secologanin and 1 mM of tryptamine.

After 6 days, 200 uL supernatant was filtered through a 0.2 μm filter membrane suitable for aquaeus solutions such as the AcroPrep™ Advance, 350 uL, 0.2 micron Supor® membrane for media/water. Next, 20 uL of 250 mg/L caffeine was added to each sample as an internal standard before analysis on the LC-MS.

Strictosidine aglycone was measured by Orbitrap Fusion™ Tribrid™ MS.

Analysis of strictosidine aglycone peaks on the Orbitrap Fusion™ Tribrid™ MS (positive mode, mass 351.1703 Da) is shown in table 4.

TABLE 4

| | Mass pos mode, 351.1703 Da | | |
|---|---|---|---|
| | Strictosidine aglycone production | | |
| | 4.08 min | 4.40 min | 4.52 min |
| MIA-BJ (EZ-Swap, full CroSTR) | N.D. | N.D. | N.D. |
| MIA-BJ + CroSGD | N.D. | N.D. | N.D. |
| MIA-BJ + RseSGD | 3.90E+06 | 7.31E+06 | 4.31E+06 |
| MIA-BJ + CroSGD + CroTHAS | N.D. | N.D. | N.D. |
| MIA-BJ + RseSGD + CroTHAS | 1.56E+06 | 2.14E+06 | 1.18E+06 |

These results show that yeast strains expressing RseSGD are able to convert secologanin and tryptamine into strictosidine aglycone. Whereas the yeast strains expressing CroSGD, alone or in combination with CroTHAS, do not produce strictosidine aglycone. This shows that RseSGD is functional in yeast, while CroSGD is not functional in yeast.

Alstonine

To further explore if yeast could be used as a microbial platform for MIA biosynthesis RseSGD and CroTHAS were co-expressed with a sapargan bridge enzymes (SBE) from either Gelsemium sempervirens (GseSBE), Catharantus roseus (CroSBE) or Rauvolfia serpentina (RseSBE), thereby enabling production of a second heteroyohimbine, alstonine.

Strain MIA-BJ (EZ-Swap, full CroSTR) expressing:
P1-TEF1-RseSGD-P2_PGK1-CroTHAS_empty vector
P1-TEF1-RseSGD-P2_PGK1-CroTHAS_P1-FET1-CroSBE
P1-TEF1-RseSGD-P2_PGK1-CroTHAS_P1-FET1-RseSBE
P1-TEF1-RseSGD-P2_PGK1-CroTHAS_P1-FET1-GseSBE

First, all strains were grown (in triplicates) in 150 uL of YPD for overnight to saturation. Then, 10 ul preculture was transferred into 500 uL of synthetic complete (SC) medium with 2% glucose, supplemented with 0.1 mM of secologanin and 1 mM of tryptamine. After 6 days, 200 uL supernatant was filtered through a 0.2 pm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure secologanin, strictosidine and tetrahydroalstonine concentrations.

The biosynthesis of the heteroyohimbine alstonine in yeast cell factories is shown in triplicates in FIG. 5. Alastonine was measured by Orbitrap Fusion™ Tribrid™ MS.

The yeast cells expressing RseSGD, CroTHAS and GseSBE were capable of converting secologanin and tryptamine to strictosidine aglycone and further capable of converting strictosidine aglycone to tetrahydroalstonine and further capable of converting tetrahydroalstonine to alstonine. This example confirms that RseSGD is functional in yeast.

### Example 6

Production of Tabersonine and Catharanthine

To further demonstrate functionalized RseSGD in yeast, the biosynthetic pathway steps from strictosidine aglycone to tabersonine and catharanthine (MIA-DC) were engineered.

Strain MIA-DC:

CroCPR+CroCYB5+CroCPR+CroCYB5+CroSTR+CroGS+RseSGD+CroGO+CroRedoc1+CroRedox2+CroSAT+CroPAS+CroCPAS+CroTS+CroCS

The MIA-DC and MIA-DA (control) strains were tested in batch fermentation using 96-well deep plate as the following.

First, all strains were grown (in triplicates) in 150 uL YPD for overnight to saturation. Then, 10 ul preculture was transferred into 500 uL of synthetic complete (SC) medium with 2% glucose, supplemented with 0.1 mM of secologanin and 1 mM of tryptamine. After 6 days, 200 uL of supernatant was filtered through a 0.2 pm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The production of tabersonine and catharanthine were measured by LC-MS.

Yeast-based production of tabersonine and catharanthine were detected, based on precursor feeding of 0.1 mM of secologanine and 1 mM of tryptamine upstream the RseSGD in strain MIA-DC (FIGS. 6A-D and 7).

### Example 7

Expanded SGD Homology Search

To further investigate, and ultimately enable, functionalization of the critical SGD node in yeast, a homology-search for SGDs against the NCO database and the PhytoMetaSyn database was performed using the RseSGD and SapSGD protein sequences as queries. From this search, 28 different SGD homologs were selected from Rauvolfia serpentina (RseSGD2), Vinca minor (VmiSGD1 and VmiSGD3), Tabernaemontana elegans (TeISGD), Amsonia hubrichtii (AhuSGD), Ophiorrhiza pumila, (OpuSGD), Nyssa sinensis, (NsiSGD1 and NsiSGD2), Coffea arabica (CarSGD), Carapichea ipecacuanha (IpeSGD), Handroanthus impetiginosus (HimSGD2 and HimSGD1), Sesamum indicum (SinSGD), Olea europaea (OeuSGD), Actinidia chinensis var. chinensis (AchSGD1, AchSGD2 and AchSGD3), Helianthus annuus (HanSGD), Lactuca sativa (LseSGD), Ipomoea nil (IniSGD), Chelidonium majus (CmaSGD), Vigna unguiculata (VunSGD), Heliocybe sulcate (HsuSGD), Pyricularia grisea (PgrSGD), Lomentospora prolificans (LprSGD), Hydnomerulius pinastri MD-312 (HpiSGD), Madurella mycetomatis (MmySGD), and Moniliophthora roreri MCA 2997 (MroSGD).

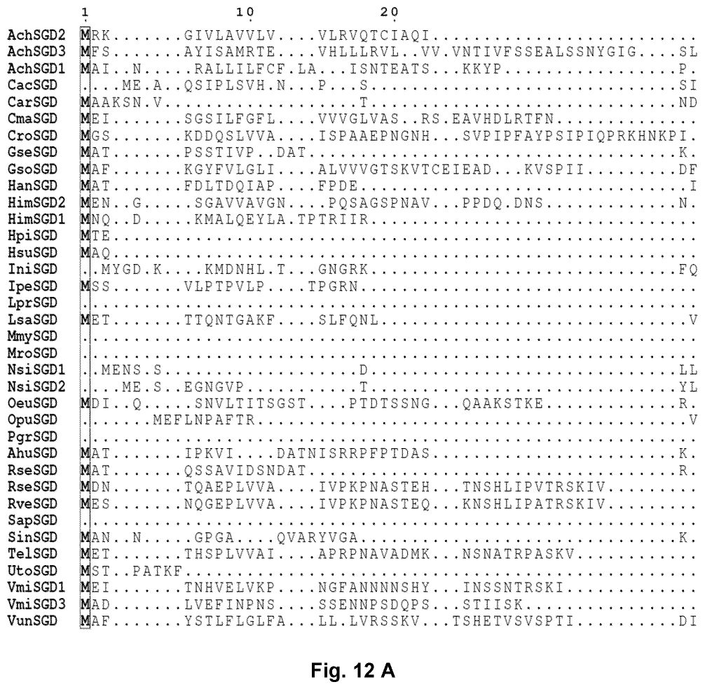

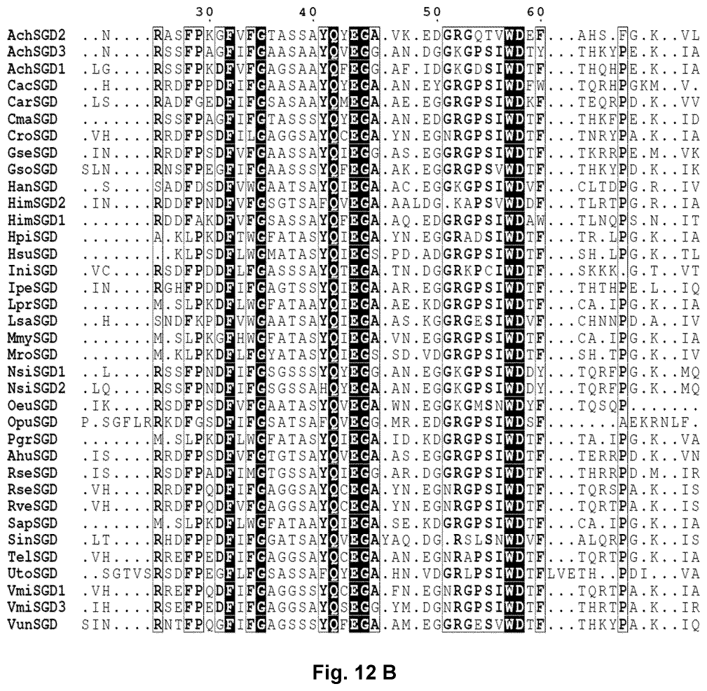

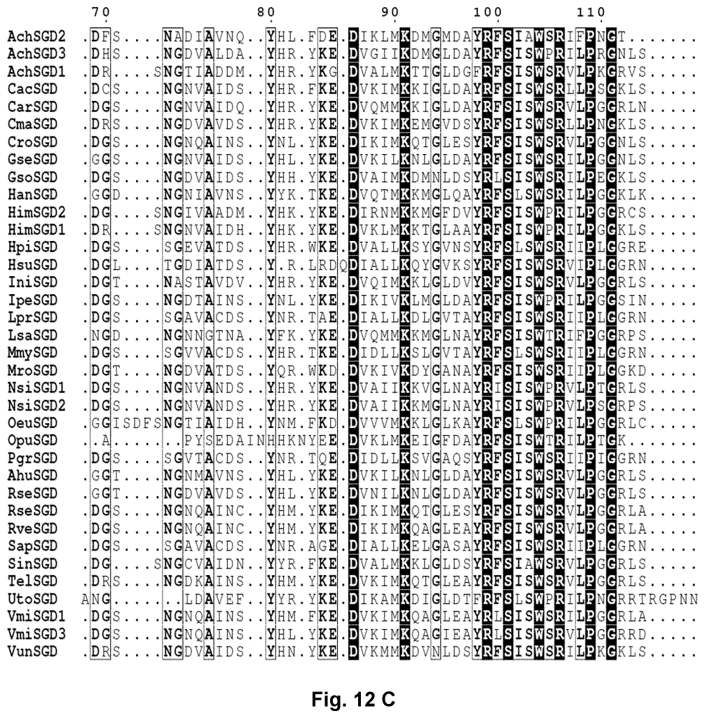

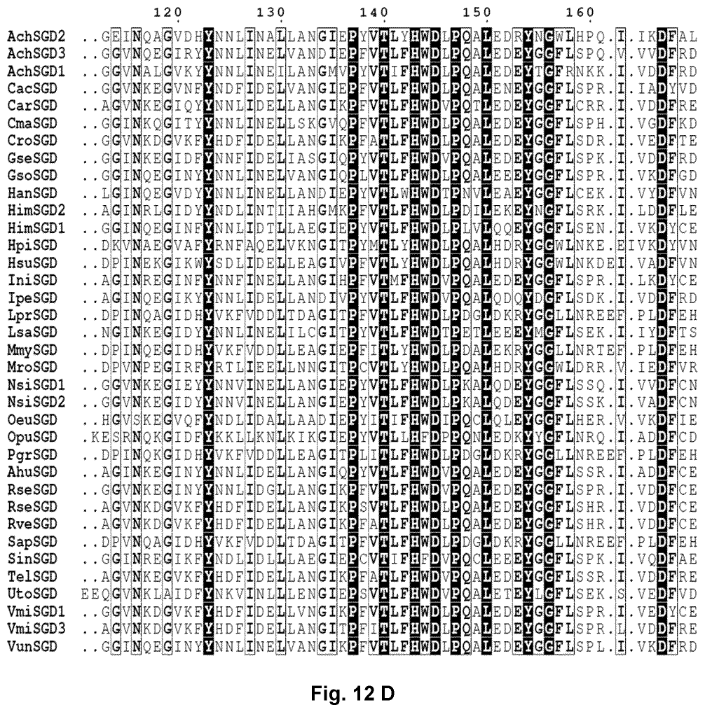

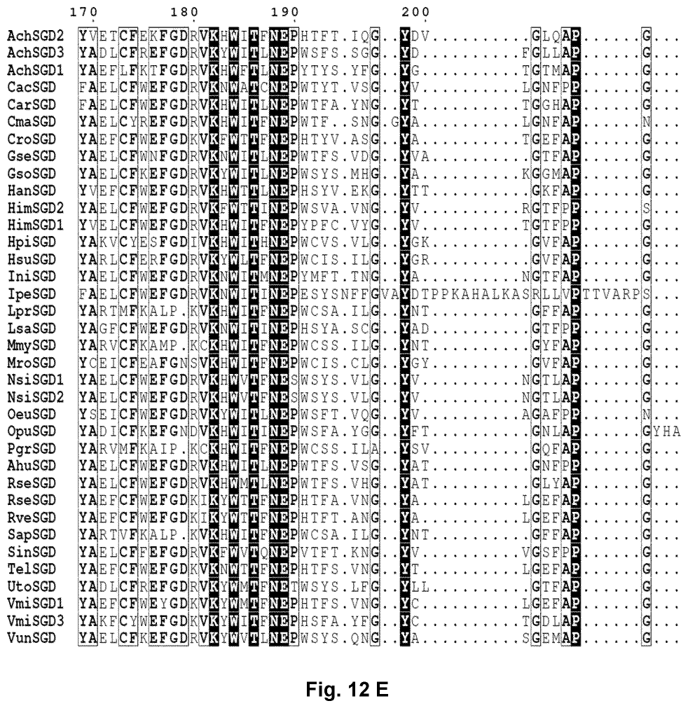

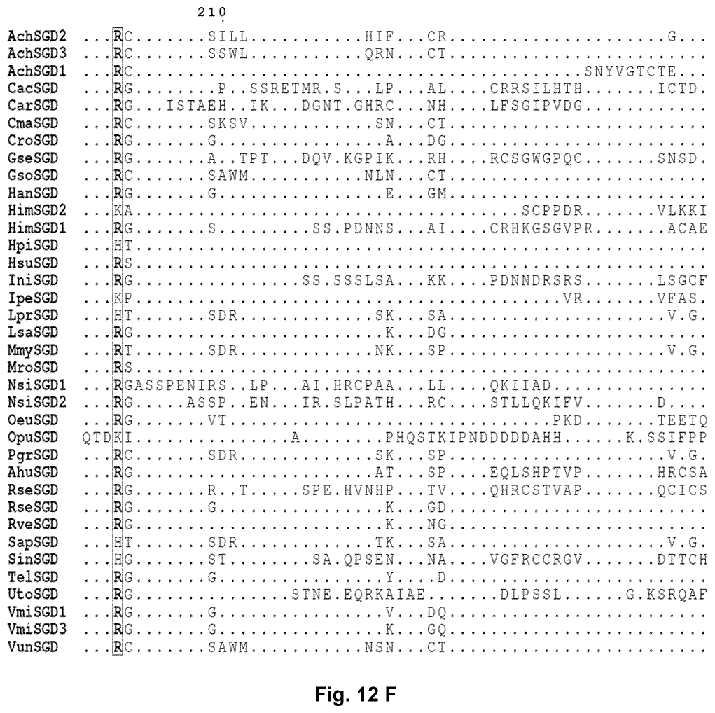

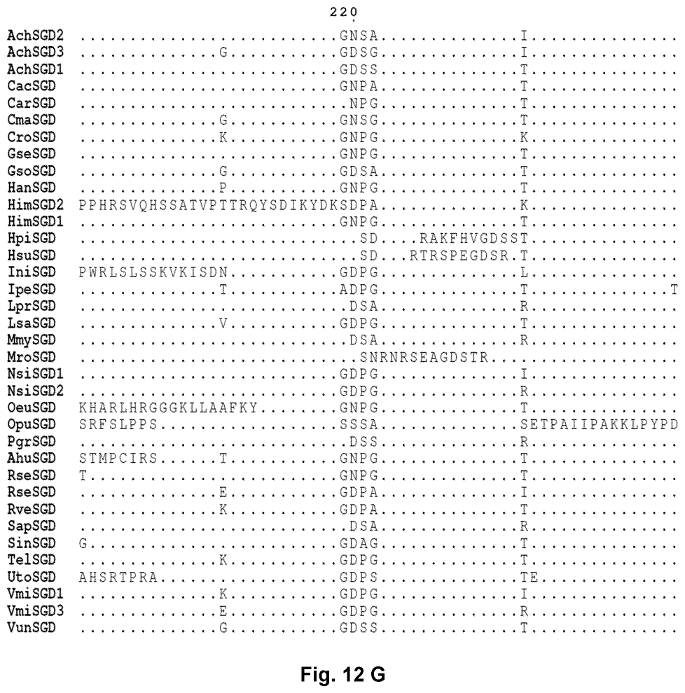

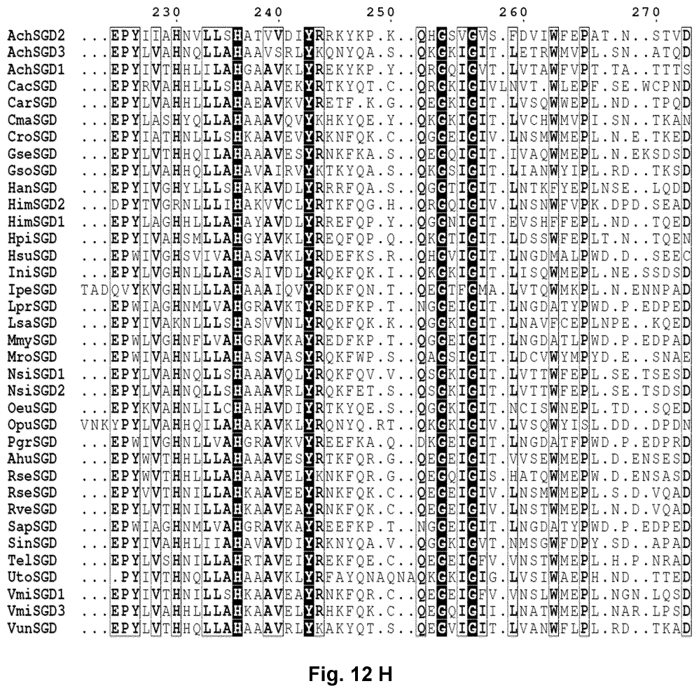

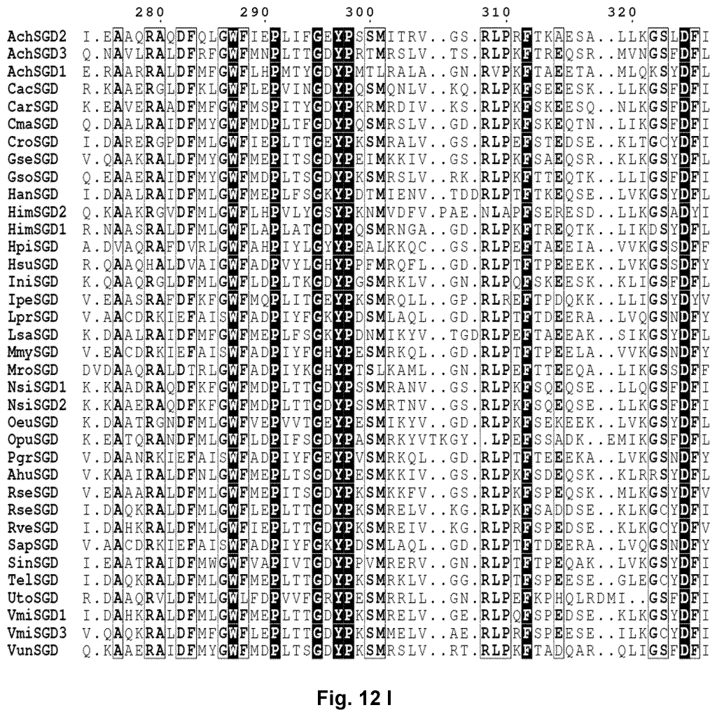

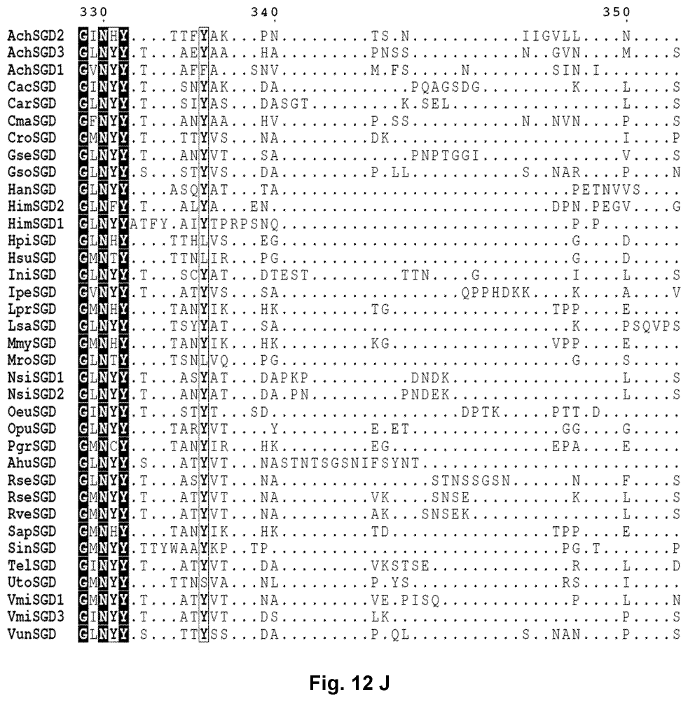

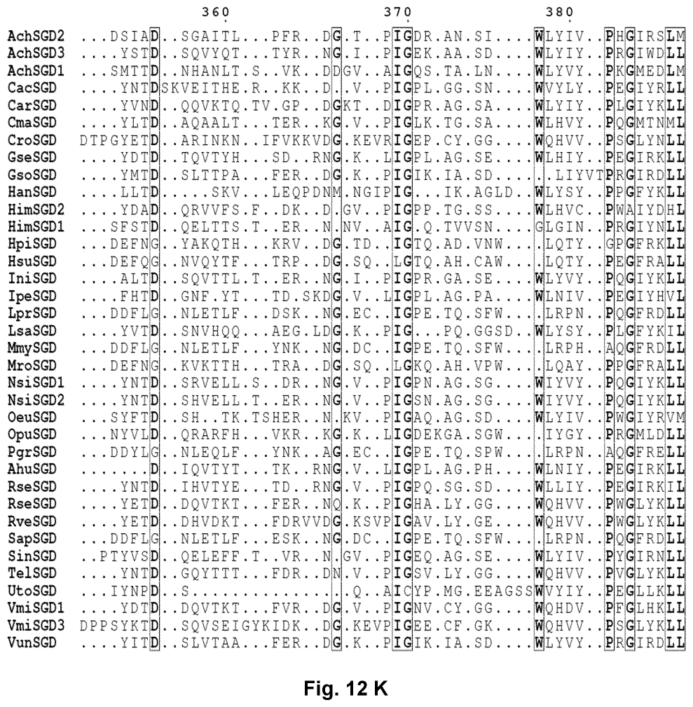

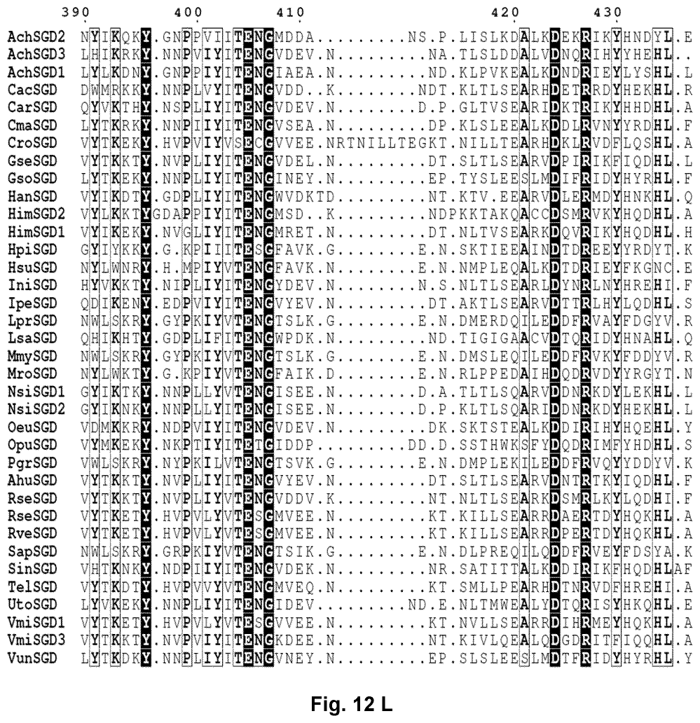

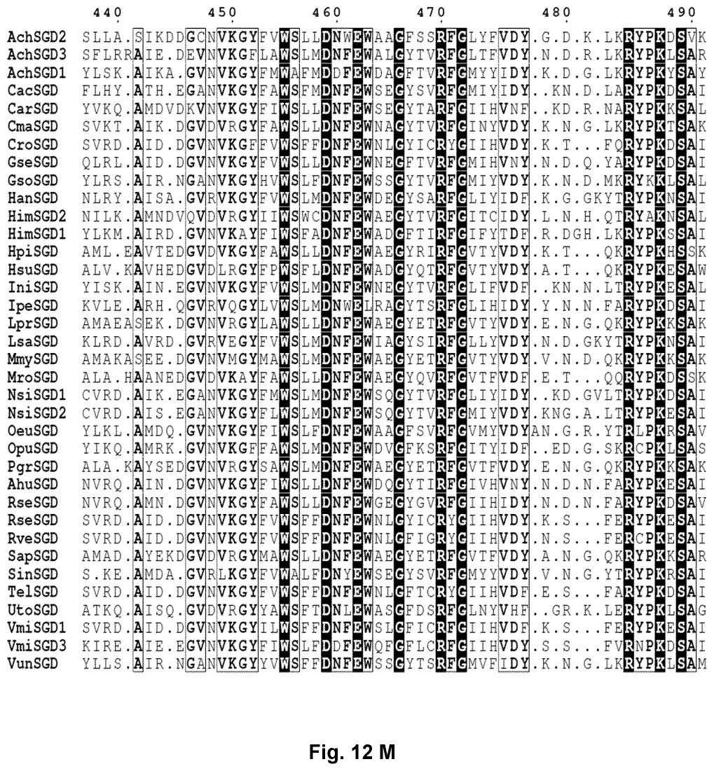

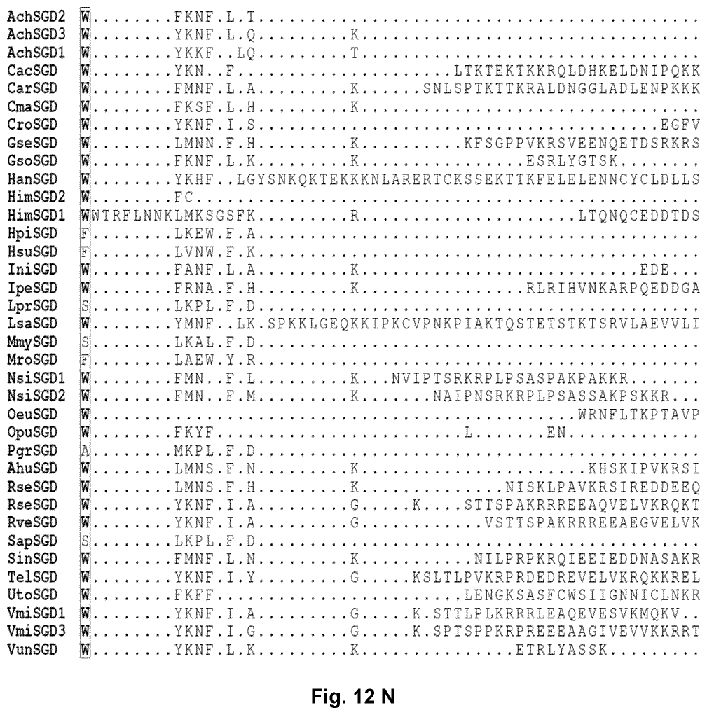

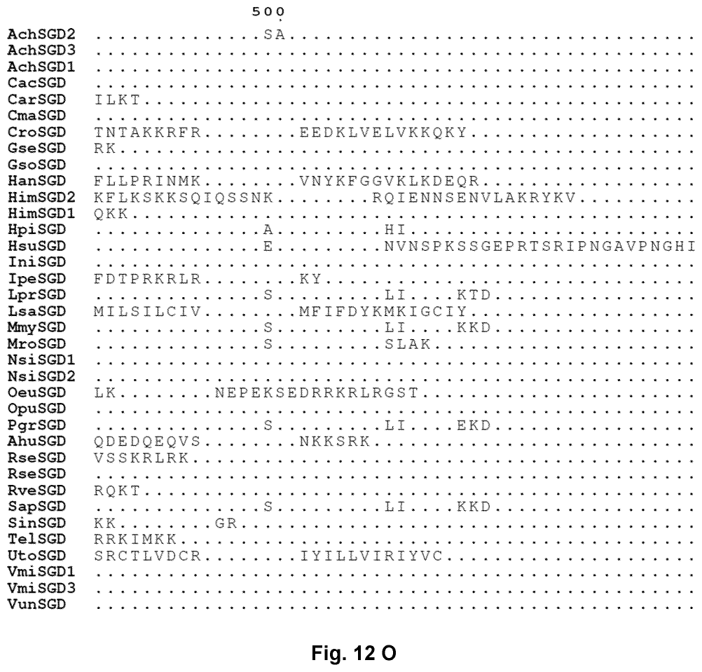

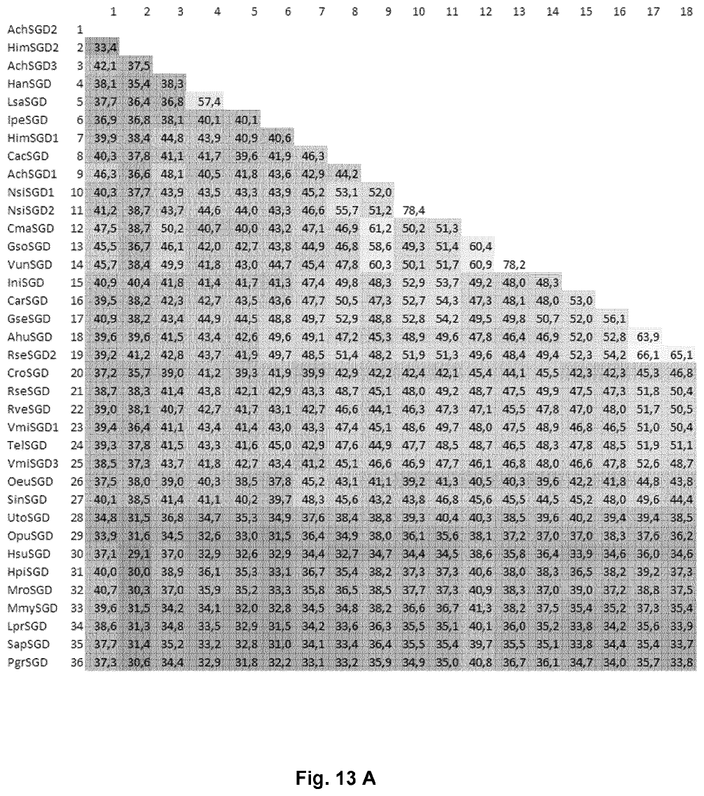

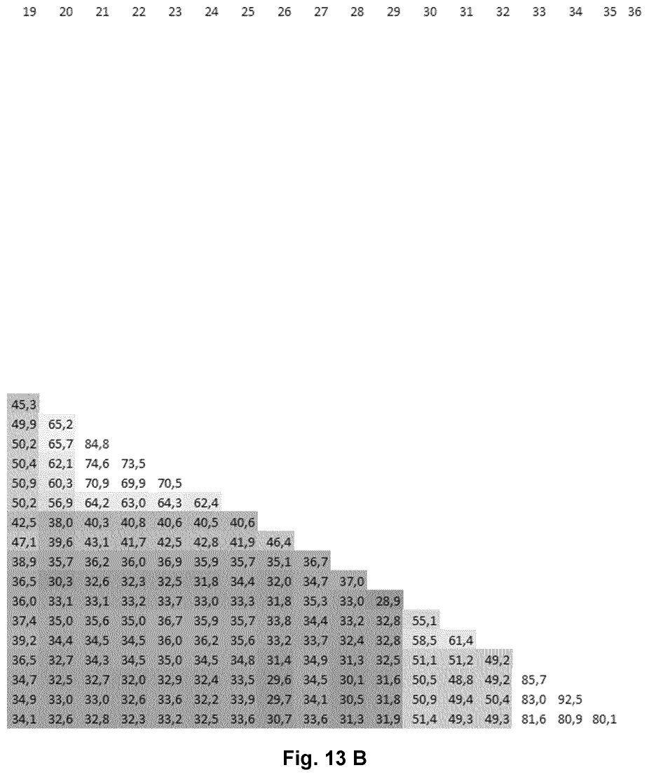

The 28 protein sequences together with RseSGD, RveSGD, CroSGD, GseSGD, CacSGD, UtoSGD, GsoSGD, and SapSGD were aligned using the t-coffee server (FIG. 12). Pairwise sequence identities were calculated from this alignment with CLC Main Workbench 8.0. (FIG. 13)

Among the 28 selected sequences for this test two (RseSGD2 and I peSGD) are known to have low SGD activity in vitro, seven are putative beta-glucosidases or hypothetical proteins from MIA producing plants (Vinca minor, Tabernaemontana elegans, Amsonia hubrichtii, Ophiorrhiza pumila, Nyssa sinensis), one (OeuSGD) is a oleuropein beta-glucosidase from Olea europaea, and 12 are putative beta-glucosidases with various putative activities from plants that do not produce MIAs but a range on different glycosylated natural products (Coffea arabica, Handroanthus impetiginosus, Sesamum indicum, Actinidia chinensis var. chinensis, Helianthus annuus, Lactuca sativa, Ipomoea nil, Chelidonium majus, and Vigna unguiculata). Six of the selected sequences are putative beta-glucosidases and hypothetical proteins from fungi (Heliocybe sulcate, Pyricularia grisea, Lomentospora prolificans, Hydnomerulius pinastri MD-312, Madurella mycetomatis, and Moniliophthora roreri MCA 2997). Nothing has been reported on glycosylated natural products produced by any of these fungi.

TABLE 5

| | | | | MIA production |
|---|---|---|---|---|
| | | | | in the origin |
| Abbreviation | Function | Species | Family | organism |
| RseSGD2 | raucaffricine- | Rauvolfia | Apocynaceae | Yes |
| | O-beta-D- | serpentina | | |
| | glucosidase | | | |
| VmiSGD1 | Putative | Vinca minor | Apocynaceae | Yes |
| | beta- | | | |
| | glucosidase | | | |
| VmiSGD3 | Putative | Vinca minor | Apocynaceae | Yes |
| | Beta- | | | |
| | glucosidase | | | |
| TelSGD | Putative | Tabernaemontana | Apocynaceae | Yes |
| | beta- | elegans | | |
| | glucosidase | | | |
| AhuSGD | Putative | Amsonia | Apocynaceae | Yes |
| | beta- | hubrichtii | | |
| | glucosidase | | | |
| OpuSGD | Putative | Ophiorrhiza | Rubiaceae | Yes |
| | beta- | pumila | | |
| | glucosidase | | | |
| NsiSGD1 | Hypothetical | Nyssa sinensis | Nyssaceae | Yes |
| | protein | | | |
| NsiSGD2 | Hypothetical | Nyssa sinensis | Nyssaceae | Yes |
| | protein | | | |
| CarSGD | Putative | Coffea arabica | Rubiaceae | No |
| | raucaffricine- | | | |
| | O-beta-D- | | | |
| | glucosidase | | | |
| IpeSGD | Beta- | Carapichea | Rubiaceae | No |
| | glucosidase | ipecacuanha | | |
| HimSGD1 | Putative | Handroanthus | Bignoniaceae | No |
| | beta- | impetiginosus | | |
| | glucosidase | | | |
| HimSGD2 | Putative | Handroanthus | Bignoniaceae | No |
| | beta- | impetiginosus | | |
| | glucosidase | | | |
| SinSGD | Putative | Sesamum | Pedaliaceae | No |
| | beta- | indicum | | |
| | glucosidase | | | |
| OeuSGD | Oleuropein | Olea europaea | Oleaceae | No |
| | beta- | | | |
| | glucosidase | | | |
| AchSGD1 | Putative | Actinidia | Actinidiaceae | No |
| | beta- | chinensis var. | | |
| | glucosidase | chinensis | | |
| AchSGD2 | Putative | Actinidia | Actinidiaceae | No |
| | beta- | chinensis var. | | |
| | glucosidase | chinensis | | |
| AchSGD3 | Putative | Actinidia | Actinidiaceae | No |
| | beta- | chinensis var. | | |
| | glucosidase | chinensis | | |
| HanSGD | Putative SGD | Helianthus | Asteraceae | No |
| | | annuus | | |
| LsaSGD | Putative | Lactuca sativa | Asteraceae | No |
| | beta- | | | |
| | glucosidase | | | |
| IniSGD | Putative | Ipomoea nil | Convolvulaceae | No |
| | raucaffricine- | | | |
| | O-beta-D- | | | |
| | glucosidase | | | |
| CmaSGD | Putative | Chelidonium | Papaveraceae | No |
| | beta- | majus | | |
| | glucosidase | | | |
| VunSGD | Putative | Vigna | Fabaceae | No |
| | cyanogenic | unguiculata | | |
| | beta- | | | |
| | glucosidase | | | |
| HsuSGD | Putative | Heliocybe | Gloeophyllaceae | No |
| | beta- | sulcata | (fungi) | |
| | glucosidase | | | |
| PgrSGD | Putative | Pyricularia | Magnaporthaceae | No |
| | lactase- | grisea | (fungi) | |
| | phlorizin | | | |
| | hydrolase | | | |
| LprSGD | Hypothetical | Lomentospora | Microascaceae | No |
| | protein | prolificans | (fungi) | |
| HpiSGD | Putative GH1 | Hydnomerulius | (fungi) | No |
| | family beta- | pinastri MD-312 | | |
| | glucosidase | | | |
| MmySGD | Putative | Madurella | (fungi) | No |
| | Beta- | mycetomatis | | |
| | glucosidase | | | |
| MroSGD | Putative | Moniliophthora | (fungi) | No |
| | beta- | roreri MCA 2997 | | |
| | glucosidase | | | |

Each one of the 28 SGD and CroSGD together with the CroHYS (capable of converting strictosidine aglycone to tetrahydroalsoinine) gene were integrated into a MIA-FA strain expressing CroG8H+Vmi8HGO-A+NcMLP+NcISY+CroCYB5+CroCPR+CrolO+CroSTR+CroSLS+Cro7DLGT+Cro7DLH+CroLAMT+CroADH2+CroHYS , resulting in strains MIA-FC-1 to MIA-FC-29. CroSGD was included as a negative control since it was already shown in example 2 to be unable to convert strictosidine to strictosidine aglycone in yeast.

MIA-FC-1: MIA-FA+CroSGD

MIA-FC-2: MIA-FA+VmiSGD1

MIA-FC-3: MIA-FA+AhuSGD

MIA-FC-4: MIA-FA+HimSGD2

MIA-FC-5: MIA-FA+SinSGD

MIA-FC-6: MIA-FA+TelSGD

MIA-FC-7: MIA-FA+VunSGD

MIA-FC-8: MIA-FA+NsiSGD1

MIA-FC-9: MIA-FA+LprSGD

MIA-FC-10: MIA-FA+AchSGD1

MIA-FC-11: MIA-FA+HsuSGD

MIA-FC-12: MIA-FA+MroSGD

MIA-FC-13: MIA-FA+RseSGD2

MIA-FC-14: MIA-FA+PgrSGD

MIA-FC-15: MIA-FA+OpuSGD

MIA-FC-16: MIA-FA+HpiSGD

MIA-FC-17: MIA-FA+HanSGD1

MIA-FC-18: MIA-FA+AchSGD2

MIA-FC-19: MIA-FA+HimSGD1

MIA-FC-20: MIA-FA+IpeSGD

MIA-FC-21: MIA-FA+LsaSGD1

MIA-FC-22: MIA-FA+CarSGD

MIA-FC-23: MIA-FA+OeuSGD

MIA-FC-24: MIA-FA+AchSGD3

MIA-FC-25: MIA-FA+CmaSGD

MIA-FC-26: MIA-FA+MmySGD

MIA-FC-27: MIA-FA+VmiSGD3

MIA-FC-28: MIA-FA+IniSGD

MIA-FC-29: MIA-FA+NsiSGD2

First, all strains were grown (in triplicates) in 150 uL of YPD overnight to saturation. Then, 10 ul preculture was transferred into 500 uL of synthetic complete (SC) medium with 2% glucose, supplemented with 0.1 mM of secologanin and 1 mM of tryptamine. After 6 days, 200 uL supernatant was filtered through a 0.2 pm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure secologanin and tetrahydroalstonine concentrations.

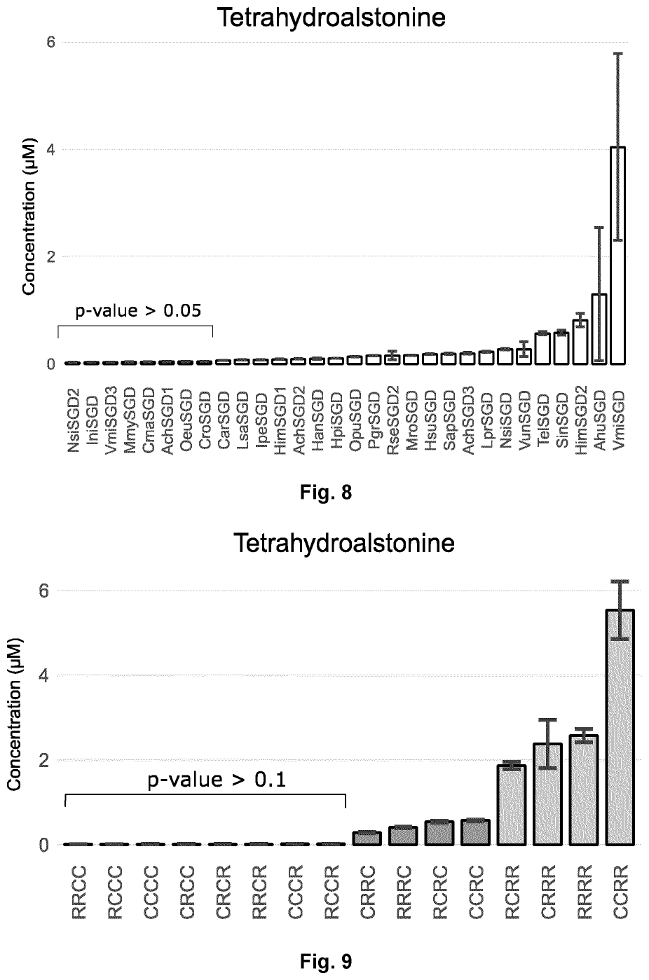

Yeast strains expressing VmiSGD1, AhuSGD, HimSGD2, SinSGD, TelSGD, VunSGD, NsiSGD1, LprSGD, AchSGD1, HsuSGD, MroSGD, RseSGD2, PgrSGD, OpuSGD, HpiSGD, HanSGD1, AchSGD2, HimSGD1, IpeSGD, LsaSGD1, and CarSGD were able to produce tetrahydroalstonine and hereby also strictosidine aglycone (FIG. 8) whereas yeast strains expressing OeuSGD, AchSGD3, CmaSGD, MmySGD, VmiSGD3, IniSGD, and NsiSGD2, as well as the negative control CroSGD were not able to produce tetrahydroalstonine. The p-value represents comparison between the negative control (CroSGD) and each of OeuSGD, AchSGD3, CmaSGD, MmySGD, VmiSGD3, IniSGD, and NsiSGD2. More homologs from MIA and non-MIA producing plants were tested, but none were able to produce tetrahydroalstonine.

### Example 8

8.1 Characterization of SGD Domains

To investigate which sequence domains are critical for SGD functionalization in yeast the protein sequences of a functional SGD (RseSGD) and a non-functional SGD (CroSGD) were aligned and divided into four domains which were then reassembled in all 16 possible combinations. The domains of RseSGD are termed R and the domains of CroSGD are termend C in this Example. Two combinations (RRRR-SGD and CCCC-SGD) corresponds to the two wild type protein sequences (RseSGD and CroSGD). The four domains are 76 to 203 amino acids long with varying sequence identity (table 6).

TABLE 6

| | Domain 1 | | Domain 2 | | Domain 3 | | Domain 4 | |
|---|---|---|---|---|---|---|---|---|
| | start | stop | start | stop | start | stop | start | stop |
| RseSGD | M1 | R115 | F116 | G266 | E267 | G456 | V457 | stop |
| | 115 | | 152 | | 190 | | 76 | |
| CroSGD | M1 | R123 | F124 | G274 | E275 | G477 | V478 | stop |

| | | | | |
|---|---|---|---|---|
| | 123 | 151 | 203 | 78 |
| Seq_ID | 63.80% | 79.60% | 64.20% | 77.60% |

Each of the 16 shuffled SGDs were cloned with USER fusion (Geu-Flores F et al. 2007) on a plasmid and transformed into a MIA-FA strain capable of expressingCroG8H+Vmi8HGO-A+NcMLP+NcISY+CroCYB5+CroCPR+CrolO+CroSTR+CroSLS+Cro7DLGT+Cro7DLH+CroLAMT+CroADH2+CroHYS, resulting in strains MIA-FD-1 to MIA-FD-16 (table 7). The MIA-FA strain is capable of synthesizing strictosidine when fed tryptamine and secologanin, or other precursors in the secologanin biosynthetic pathway from geraniol, and is also capable of converting strictosidine aclycone to tetrahydroalstonine if a functional SGD capable of converting strictosidine to strictosidine aglycone is coexpressed.

TABLE 7

| Strain | Domain 1 | Domain 2 | Domain 3 | Domain 4 |
|---|---|---|---|---|
| MIA-FD-1: MIA-FA + | CroSGD | CroSGD | CroSGD | CroSGD |
| pRS413U_pTEF1_CCCC-SGD | | | | |
| MIA-FD-2: MIA-FA + | CroSGD | RseSGD | CroSGD | CroSGD |
| pRS413U_pTEF1_CRCC-SGD | | | | |
| MIA-FD-3: MIA-FA + | CroSGD | RseSGD | CroSGD | RseSGD |
| pRS413U_pTEF1_CRCR-SGD | | | | |
| MIA-FD-4: MIA-FA + | CroSGD | CroSGD | CroSGD | RseSGD |
| pRS413U_pTEF1_CCCR-SGD | | | | |
| MIA-FD-5: MIA-FA + | CroSGD | RseSGD | RseSGD | CroSGD |
| pRS413U_pTEF1_CRRC-SGD | | | | |
| MIA-FD-6: MIA-FA + | CroSGD | CroSGD | RseSGD | RseSGD |
| pRS413U_pTEF1_CCRC-SGD | | | | |
| MIA-FD-7: MIA-FA + | CroSGD | RseSGD | RseSGD | RseSGD |
| pRS413U_pTEF1_CRRR-SGD | | | | |
| MIA-FD-8: MIA-FA + | CroSGD | CroSGD | RseSGD | RseSGD |
| pRS413U_pTEF1_CCRR-SGD | | | | |
| MIA-FD-9: MIA-FA + | RseSGD | RseSGD | CroSGD | CroSGD |
| pRS413U_pTEF1_RRCC-SGD | | | | |
| MIA-FD-10: MIA-FA + | RseSGD | CroSGD | CroSGD | CroSGD |
| pRS413U_pTEF1_RCCC-SGD | | | | |
| MIA-FD-11: MIA-FA + | RseSGD | RseSGD | CroSGD | RseSGD |
| pRS413U_pTEF1_RRCR-SGD | | | | |
| MIA-FD-12: MIA-FA + | RseSGD | CroSGD | CroSGD | RseSGD |
| pRS413U_pTEF1_RCCR-SGD | | | | |
| MIA-FD-13: MIA-FA + | RseSGD | RseSGD | RseSGD | CroSGD |
| pRS413U_pTEF1_RRRC-SGD | | | | |
| MIA-FD-14: MIA-FA + | RseSGD | CroSGD | RseSGD | CroSGD |
| pRS413U_pTEF1_RCRC-SGD | | | | |
| MIA-FD-15: MIA-FA + | RseSGD | CroSGD | RseSGD | RseSGD |
| pRS413U_pTEF1_RCRR-SGD | | | | |
| MIA-FD-16: MIA-FA + | RseSGD | RseSGD | RseSGD | RseSGD |
| pRS413U_pTEF1_RRRR-SGD | | | | |

First, all strains were grown (in triplicates) in 150 uL of synthetic complete without histidine (SC-HIS) overnight to saturation. Then, 10 ul preculture was transferred into 500 uL of SC-HIS medium with 2% glucose, supplemented with 0.1 mM of secologanin and 1 mM of tryptamine. After 6 days, 200 uL supernatant was filtered through a 0.2 pm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure secologanin tetrahydroalstonine concentrations.

Results

Yeast strains expressing CRRC-SGD, RRRC-SGD, RCRC-SGD, CCRC-SGD, CRRR-SGD, CCRR-SGD, RCRR-SGD, and RRRR-SGD were able to produce tetrahydroalstonine (FIG. 9). All functional SGD variants have RseSGD domain 3. All SGD variants with CroSGD domain 3 were not able to produce tetrahydroalstonine. The identity of domain 1 and 2 has low or no effect. Of the functional SGD variants, the four sequences with RseSGD domain 3 and domain 4 (CRRR-SGD, CCRR-SGD,

RCRR-SGD, and RRRR-SGD) are able to produce the highest amount of tetrahydroalstonine. CCRR-SGD is the best variant capable of producing more tetrahydroalstonine than the wild type RseSGD (RRRR-SGD)

8.2 Production of Tetrahydroalstonine in a Yeast Strain Expressing CCRR_SGD

The best SGD variant (CCRR-SGD) were integrated in the MIA-FA strain MIA-FA capable of strain expressing CroG8H+Vmi8HGO-A+NcMLP+NcISY+CroCYB5+CroCPR+CrolO+CroSTR+CroSLS+Cro7DLGT+Cro7DLH+CroLAMT+CroADH2+CroHYS, resulting in the strain MIA-FE:

MIA-FE: MIA-FA+CCRR-SGD

First, MIA-FE was grown (in triplicates) in 150 uL of YPD overnight to saturation. Then, 10 ul preculture was transferred into 500 uL of synthetic complete (SC) medium with 2% glucose, supplemented with 0.1 mM of secologanin and 1 mM of tryptamine. After 6 days, 200 uL supernatant was filtered through t a 0.2 μm filter membrane suitable for aquaeus solutions such as he AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure tetrahydroalstonine concentrations.

Results

The yeast strain expressing CCRR-SGD was able to produce 13.30 μM (±1.29 μM) tetrahydroalstonine.

### Example 9

Rescuing the function of other SGD homologs with RseSGD domain 3 and 4

Encouraged by the capability of RseSGD domain 3 and 4 to rescue the non-functional CroSGD in yeast three more SGD variants were cloned swapping domain 3 and 4 between RseSGD and UtoSGD (U), GseSGD (G), and RveSGD (V) respectively. Even though swapping domain 3 alone was able to make CroSGD functional swapping both domain 3 and domain 4 gave the largest improvement and therefor this swapping strategy was expanded to other SGD sequences.

The sequences of the four domains of UtoSGD, GseSGD and RveSGD were determined from a multiple sequence alignment (FIG. 12). The first residue in domain 1 is always the start methionine and the last residue in domain 4 is always the last residue in the sequence. The remaining first and last residues are defined as the residues aligning with the first and last residues in the four RseSGD domains. Table 8 summarizes the four domains of RseSGD, CroSGD, UtoSGD, GseSGD, and RveSGD.

TABLE 8

| | Domain 1 | | Domain 2 | | Domain 3 | | Domain 4 | | Seq_ID to |
|---|---|---|---|---|---|---|---|---|---|
| | start | stop | start | stop | start | stop | start | stop | RseSGD |
| RseSGD | M1 | R115 | F116 | G266 | E267 | G456 | V457 | stop | |
| UtoSGD | M1 | R88 | F89 | G277 | K278 | G459 | V460 | stop | 40.70% |
| GseSGD | M1 | R92 | F93 | G265 | Q266 | G456 | V457 | stop | 53.90% |
| CroSGD | M1 | R123 | F124 | G274 | E275 | G477 | V478 | stop | 70.30% |
| RveSGD | M1 | R115 | F116 | G265 | E266 | G459 | V460 | stop | 89.90% |

Three domain-swap SGD variants and the three wild type SGDs were cloned with USER fusion. The plasmids were transformed into a MIA-FA strain capable of expressing CroG8H+Vmi8HGO-A+NcMLP+NcISY+CroCYB5+CroCPR+CrolO+CroSTR+CroSLS+Cro7DLGT+Cro7DLH+CroLAMT+CroADH2+CroHYS, resulting in strains MIA-FD-17 to MIA-FD-22 (table 9). The MIA-FA strain is capable of synthesizing strictosidine when fed tryptamine and secologanin, or other precursors in the secologanin biosynthetic pathway from geraniol, and is also capable of converting strictosidine aclycone to tetrahydroalstonine if a functional SGD capable of converting strictosidine to strictosidine aglycone is coexpressed

TABLE 9

| | | | | |
|---|---|---|---|---|
| MIA-FD-17: MIA-FA + | UtoSGD | UtoSGD | UtoSGD | UtoSGD |
| pRS413U_pTEF1_UtoSGD-SGD | | | | |
| MIA-FD-18: MIA-FA + | UtoSGD | UtoSGD | RseSGD | RseSGD |
| pRS413U_pTEF1_UURR-SGD | | | | |
| MIA-FD-19: MIA-FA + | GseSGD | GseSGD | GseSGD | GseSGD |
| pRS413U_pTEF1_GseSGD-SGD | | | | |
| MIA-FD-20: MIA-FA + | GseSGD | GseSGD | RseSGD | RseSGD |
| pRS413U_pTEF1_GGRR-SGD | | | | |
| MIA-FD-21: MIA-FA + | RveSGD | RveSGD | RveSGD | RveSGD |
| pRS413U_pTEF1_RveSGD-SGD | | | | |
| MIA-FD-22: MIA-FA + | RveSGD | RveSGD | RseSGD | RseSGD |
| pRS413U_pTEF1_VVRR-SGD | | | | |

First, all six strains plus two control strains (MIA-FD-1 and 8) were grown (in triplicates) in 150 uL of synthetic complete without histidine (SC-HIS) overnight to saturation. Then, 10 ul preculture was transferred into 500 uL of SC-HIS medium with 2% glucose, supplemented with 0.1 mM of secologanin and 1 mM of tryptamine. After 6 days, 200 uL supernatant was filtered through a 0.2 pm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure tetrahydroalstonine concentrations.

As already shown in example 9, swapping in RseSGD domain 3 and 4 rescued the function of the non-functional CroSGD (FIG. 9). Wild type RveSGD is capable of producing tetrahydroalstonine. Swapping in RseSGD domain 3 and 4 improved the tetrahydroalstonine production about seven fold. GseSGD and UtoSGD have lower sequence identity to RseSGD (53.9% and 40.7% respectively) than CroSGD and RveSGD (70.3% and 89.9%). GseSGD can produce tetrahydroalstonine in low concentrations whereas UtoSGD is incapable of tetrahydroalstonine production. Swapping in RseSGD domain 3 and 4 into these two SGDs did not rescue the function of UtoSGD and abolished the low tetrahydroalstonine production of GseSGD.

### Example 10

Minimum Strictosidine Aglycone Production in Yeast

Strictosidine aglycone is chemically unstable and was impossible to either purchase or purify to use as a standard for quantification. The minimum strictosidine aglycone produced by the tested SGD homologs was calculated from the measured tetrahydroalstonine produced by the yeast strains and the measured secologanin left in the media. It is possible that not all produced strictosidine aglycone is converted to tetrahydroalstonine, and therefore the true strictosidine aglycone titres might in some cases be higher than the estimated minimum production.

Strictosidine Aqlycone Production in μM:

Since strictosidine aglycone is converted to tetrahydroalstonine in equimolar amounts, the minimum strictosidine aglycone titre equals the tetrahydroalstonine titre.

c(strictosidine aglycone)=c(tetrahydroalstonine)

Strictosidine Alycone Yields:

The minimum strictosidine algycone yield can be estimated from the strictosidine aglycone titre and the theoretical strictosidine titre. It is assumed that all secologanin taken up by the yeast strain is converted to strictosidine.

Strictosidine_aglycone_%=c(strictosidine aglycone)/(c(secologanin supplemented in media)−c(secologanin left after cultivation))

### Example 11

Production of THA in Escherichia coli

To test if RseSGD or CroSGD could be used for production of strictosidine aglycone and MIAs in prokaryotic microorganisms an expression system was established in the gram-negative bacterium Escherichia coli for in vivo conversion of secologanin and tryptamine to strictosidine by CroSTR, conversion of strictosidine to strictosidine aglycone by RseSGD or CroSGD and conversion of strictosidine aglycone to tetrahydroalstonine by CroHYS. Two low-copy plasmids were cloned for co-expression of the three genes from a polycistronic mRNA under control of a medium strength constitutive promoter. The plasmids were based on pCfB3510(p15A_P2BCD2GFP).

The two plasmids and an empty plasmid were transformed into the strain DH5-α giving the three strains MIA-ECO-1 to MIA-ECO-3.

MIA-ECO-1: D H5-α+p15A-AmpR-CroSTR-CroHYS-CroSGD

MIA-ECO-2: DH5-α+p15A-AmpR-CroSTR-CroHYS-RseSGD

MIA-ECO-3: DH5-α+p15A-AmpR

First, all three strains were grown (in triplicates) in 150 uL of Lysogeny broth (LB) medium with 100 μg/mL ampicillin overnight to saturation. Then, 10 ul preculture was transferred into 500 uL LB medium with 100 μg/mL ampicillin and supplemented with 0.1 mM of secologanin and 1 mM of tryptamine. After 48 hours, 200 uL supernatant was filtered through a 0.2 μm filter membrane suitable for aquaeus solutions such as the AcroPrep™ Advance, 350 uL, 0.2 micron Supor® membrane for media/water. Next, 20 uL of 250 mg/L caffeine was added to each sample as internal standard before analysis on the LC-MS.

The sample caffeine mixtures were analysed on LC-MS to measure secologanin, strictosidine, and tetrahydroalstonine concentrations.

Results

The E. coli strain MIA-ECO-2 expressing RseSGD, CroSTR, and CroHYS was able to produce tetrahydroalstonine (FIG. 11-B). No strictosidine was detected in the media of the E. coli expressing RseSGD. MIA-ECO-1 expressing CroSGD, CroSTR, and CroHYS produced strictosidine (FIG. 11-A) but no tetrahydroalstonine, indicating that like in yeast RseSGD is functional and CroSGD is non-functional.

### REFERENCES

Geerlings, A., Ibanez, M. M., Memelink, J., van Der Heijden, R. & Verpoorte, R. Molecular cloning and analysis of strictosidine beta-D-glucosidase, an enzyme in terpenoid indole alkaloid biosynthesis in Catharanthus roseus. J. Biol. Chem. 275, 3051-3056 (2000).

Fernando Geu-Flores, Hussam H. Nour-Eldin, Morten T. Nielsen and Barbara A. Halkier 2007. USER fusion: a rapid and efficient method for simultaneous fusion and cloning of multiple PCR products. Nucleic Acids Research, 2007, Vol. 35, No. 7 e55. doi:10.1093/nar/gkm106

Guirimand G., Courdavault V., Lanoue A., Mahroug S., Guihur A., Blanc N., Giglioli-Guivarc'h N., St-Pierre B., Burlat V. Strictosidine activation in Apocynaceae: towards a “nuclear time bomb”? BMC Plant Biology 2010, 10:182

Jakoc̆iūnas T, Rajkumar A S, Zhang J, Arsovska D, Rodriguez A, Jendresen C B, Skjodt M L, Nielsen A T, Borodina I, Jensen M K, Keasling J D. CasEMBLR: Cas9-Facilitated Multiloci Genomic Integration of in Vivo Assembled DNA Parts in Saccharomyces cerevisiae. ACS Synth Biol. 2015 Nov. 20; 4(11):1226-34. doi: 0.1021/acssynbio.5b00007. Epub 2015 Mar. 26.

Jensen N B, Strucko T, Kildegaard K R, David F, Maury J, Mortensen U H, Forster J, Nielsen J, Borodina I. EasyClone: method for iterative chromosomal integration of multiple genes in Saccharomyces cerevisiae. FEMS Yeast Res. 2014 March; 14(2):238-48. doi: 10.1111/1567-1364.12118. Epub 2013 Nov. 18.

Luijendick T. J. C., Stenvens, L. H., Verpoorte R. Reaction for the Localization of Strictosidine Glucosidase Activity on Polyacrylamide gels. Phytochemical analysis (1996). doi:3.0.00;2-H″>10.1002/(SICI)1099-1565(199601)7:1<16::AID-PCA280>3.0.CO; 2-H.

Stavrinides A., Tatsis E. C., Foureau E., Caputi L., Kellner F., Courdavault V., O'Connor S. E. Unlocking the Diversity of Alkaloids in Catharanthus roseus: Nuclear Localization Suggests Metabolic Channeling in Secondary Metabolism. Chemistry & Biology 22, 336-341, Mar. 19, 2015

Steyaert J, Van Melderen L, Bernard P, Thi M H, Loris R, Wyns L, Couturier M. J Mol Purification, circular dichroism analysis, crystallization and preliminary X-ray diffraction analysis of the F plasmid CcdB killer protein Biol. 1993 May 20; 231(2):513-5.

WO 00/4220: Verpoorte, R., Van Der Heijden, R., Memelink, J. & Geerlings, A. Strictosidine glucosidase from Catharanthus roseus and its use in alkaloid production. World Patent (2000).

Items
1. A microorganism capable of producing strictosidine aglycone, said microorganism expresses
   a strictosidine-beta-glucosidase (SGD), capable of converting strictosidine to strictosidine aglycone,
   wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
   and/or;
   wherein said SGD is a mosaic SGD, wherein said mosaic SGD comprises an amino acid sequence having the general formula

D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,
2. wherein said first SGD, second SGD and fourth SGD can be the same or different, with the proviso that said first SGD, second SGD and fourth SGD are not all RseSGD. The microorganism according to item 1, further expressing
   a strictosidine synthase (STR), capable of converting secologanin and tryptamine to strictosidine, whereby the microorganism is capable of synthesising strictosidine,
   wherein said STR is preferably CroSTR or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 30.
3. The microorganism according to any one of the preceding items, wherein D₁ comprises or consists of an amino acid sequence corresponding to amino acids M1 to R115 of SEQ ID NO:24.
4. The microorganism according to any one of the preceding items, wherein D₂ comprises or consists of an amino acid sequence corresponding to amino acids F116 to G266 of SEQ ID NO:24.
5. The microorganism according to any one of the preceding items, wherein D₄ comprises or consists of amino acids of SEQ ID NO:92 or a variant thereof having at least 90% identity to SEQ ID NO: 92.
6. The microorganism according to any one of the preceding items, wherein at least one of D₁, D₂ or D₄ is from an SGD which is native to a first organism selected from Gelsemium sempervirens, Scedosporium apiospermum or Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 2997.
7. The microorgagnism according to any one of the preceding items, wherein the first SGD, the second SGD and the fourth SGD are identical or different.
8. The microorganism according to any one of the preceding items, wherein two of the first SGD, the second SGD and the fourth SGD are identical, or wherein the first SGD, the second SGD and the fourth SGD are different, or wherein the first SGD, the second SGD and the fourth SGD are identical.
9. The microorganism according to any one of the preceding items, wherein said mosaic SGD comprises or consists of an amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99, or SEQ ID NO: 108, or variants thereof having at least 90% identity or homology thereto, such as at least 91%, such as at least 92%, such as at least 93%, such as at least 94%, such as at least 95%, such as at least 96%, such as at least 97%, such as at least 98%, such as at least 99% identity or homology thereto.
10. The microorganism according to any one the preceding items, further expressing
   a tetrahydroalstonine synthase (THAS) and/or a heteroyohimbine synthase (HYS), capable of converting strictosidine aglycone to tetrahydroalstonine, whereby the microorganism is capable of synthesising tetrahydroalstonine,
   wherein said THAS is preferably CroTHAS and/or HYS is CroHYS or variants thereof, having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 28 and/or SEQ ID NO: 46.
11. The microorganism according to any of the preceding items, further expressing
   a sarpargan bridge enzymes (SBE), capable of converting tetrahydroalstonine and ajmalicine to a heteroyohimbine selected from the group consisting of alstonine and serpentine, whereby the microorganism is capable of synthesising alstonine and serpentine,
   wherein said SBE is preferably GseSBE or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 29.
12. The microorganism according to any one of the preceding items, further expressing
   a NADPH-cytochrome P450 reductase (CPR);
   a Cytochrome b5 (CYB5);
   a Geissoschizine synthase (GS);
   a Geissoschizine oxidase (GO);
   a Redox1;
   a Redox2;
   a Stemmadenine O-acetyltransferase (SAT);
   a O-acetylstemmadenine oxidase (PAS);
   a Dehydroprecondylocarpine acetate synthase (DPAS);
   a Tabersonine synthase (TS); and/or
   a Catharanthine synthase (CS),
   whereby the microorganism is capable of synthesising tabersonine and/or catharanthine,
   wherein preferably said CPR is CroCPR, said CYB5 is CroCYB5, said GS is CroSG, said GO is CroGO, said Redox1 is CroRedox1, said Redox2 is CroRedox2, said SAT is CroSAT, said PAS is CroPAS, said DPAS is CroDPAS, said TS is CroTS and/or said CS is CroCS or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 and/or SEQ ID NO: 41, respectively.
13. The microorganism according to any one of the preceding items, capable of producing strictosidine aglycone with a titre of at least 1 μM, such as at least 2 μM, such as at least 4 μM, such as at least 6 μM, such as at least 8 μM such as at least 10 μM or more.
14. The microorganism according to item 10, capable of producing tetrahydroalstonine with a titre of at least 1 μM, such as at least 2 μM, such as at least 4 μM, such as at least 6 μM, such as at least 8 μM such as at least 10 μM or more.
15. The microorganism according to item 11, capable of producing alstonine with a titre of at least 1 μM, such as at least 2 μM, such as at least 4 μM, such as at least 6 μM, such as at least 8 μM such as at least 10 μM or more.
16. The microorganism according to item 12, capable of producing tabersonine with a titre of at least 0.01 μM, such as at least 0.02 μM.
17. The microorganism according to item 12, capable of producing catharanthine with a titre of at least 0.01 μM, such as at least 0.02 μM.
18. The microorganism according to any of the preceding items, wherein the microorganism is selected from the group consisting of yeasts, bacteria, archaea, fungi, protozoa, algae, and viruses, preferably the microorganism is a yeast or a bacteria.
19. The microorganism according to any one of the preceding items, wherein the microorganism is a bacteria.
20. The microorganism according to item 19, wherein the genus of said bacteria is selected from the groups consisting of Escherichia, Corynebacterium, Pseudomonas, Bacillus, Lactococcus, Lactobacillus, Halomonas, Bifidobacterium and Enterococcus.
21. The microorganism according to any one of items 19 to 20, wherein the bacteria is selected from the group consisting of Escherichia coli, Corynebacterium glutamicum, Pseudomonas putida, Bacillus subtilis, Lactococcus bacillus, Halomonas elongate, Bifidobacterium infantis and Enterococcus faecal.
22. The microorganism according to any one of items 19 to 21, wherein the bacteria is Escherichia coli.
23. The microorganism according to any one of the preceding items, wherein the microorganism is a yeast.
24. The microorganism according to item 23, wherein the genus of said yeast cell is selected from the group consisting of Saccharomyces, Pichia, Yarrowia, Kluyveromyces, Candida, Rhodotorula, Rhodosporidium, Cryptococcus, Trichosporon and Lipomyces.
25. The microorganism according to any one of items 23 to 24, wherein the yeast is selected from the group consisting of Saccharomyces cerevisiae, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica
26. The microorganism according to any one of items 23 to 25, wherein the yeast is Saccharomyces cerevisiae.
27. The microorganism according to any of the preceding items, wherein the microorganism comprises a nucleic acid encoding SGD, said nucleic acid having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 1.
28. A method of producing strictosidine aglycone in a microorganism, said method comprising the steps of:
   a) providing a microorganism, said cell expressing:
      a strictosidine-beta-glucosidase (SGD), capable of converting strictosidine to strictosidine aglycone;
   b) incubating said microorganism in a medium comprising strictosidine or a substrate which can be converted to strictosidine by said microorganism;
   c) optionally, recovering the strictosidine aglycone;
   d) optionally, further converting the strictosidine aglycone to monoterpenoid indole alkaloids,
   wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
   and/or;
   wherein said SGD is a mosaic SGD, wherein said mosaic SGD comprises an amino acid sequence having the general formula

D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,
wherein said first SGD, second SGD and fourth SGD can be the same or different, with the proviso that said first SGD, second SGD and fourth SGD are not all RseSGD.
29. The microorganism according to item 28, wherein the SGD, the heterologous SGD and/or the mosaic SGD is as defined in any one of the preceding items.
30. The microorganism according to any one of items 28 to 29, wherein D₁ comprises or consists of an amino acid sequence corresponding to amino acids M1 to R115 of SEQ ID NO:24.
31. The microorganism according to any one of items 28 to 30, wherein D2 comprises or consists of an amino acid sequence corresponding to amino acids F116 to G266 of SEQ ID N0:24.
32. The microorganism according to any one of items 28 to 31, wherein D₄ comprises or consists of amino acids of SEQ ID NO:92 or a variant thereof having at least 90% identity to SEQ ID NO: 92.
33. The microorganism according to any one of items 28 to 32, wherein at least one of D₁, D₂ or D₄ is from an SGD which is native to a first organism selected from Gelsemium sempervirens, Scedosporium apiospermum or Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 2997.
34. The microorgagnism according to any one of items 28 to 33, wherein the first SGD, the second SGD and the fourth SGD are identical or different.
35. The microorganism according to any one of items 28 to 34, wherein two of the first SGD, the second SGD and the fourth SGD are identical, or wherein the first SGD, the second SGD and the fourth SGD are different, or wherein the first SGD, the second SGD and the fourth SGD are identical.
36. The microorganism according to items 28 to 35, wherein said mosaic SGD comprises or consists of an amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99, or SEQ ID NO: 108, or variants thereof having at least 90% identity or homology thereto, such as at least 91%, such as at least 92%, such as at least 93%, such as at least 94%, such as at least 95%, such as at least 96%, such as at least 97%, such as at least 98%, such as at least 99% identity or homology thereto.
37. The method according to any one of items 28 to 36, wherein the substrate is secologanin and/or tryptamine, and wherein said microorganism further expresses:
   a strictosidine synthase (STR), capable of converting secologanin and tryptamine to strictosidine;
   wherein said STR is preferably CroSTR or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 30.
38. The method according to any one of items 28 to 37, wherein the method comprising step d) and wherein said microorganism further expresses:
   a tetrahydroalstonine synthase (THAS) and/or or a heteroyohimbine synthase (HSY), capable of converting strictosidine aglycone to tetrahydroalstonine;
   wherein preferably said THAS is identical to or has at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 28 and/or HYS is identical to or has at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 46.
39. The method according to items 28 to 38, wherein said method further comprises the step of recover tetrahydroalstonine.
40. The method according to any one of items 28 to 39, wherein the method comprising step d) and wherein said microorganism further expresses:
   a sapargan bridge enzyme (SBE), capable of converting tetrahydroalstonine to alstonine;
   wherein preferably said SBE is identical to or has at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 29.
41. The method according to item 40, wherein said method further comprises the step of recovering alstonine.
42. The method according to any one of items 28 to 41, wherein the method comprises step d) and wherein said microorganism further expresses:
   a NADPH-cytochrome P450 reductase (CPR);
   a Cytochrome b5 (CYB5);
   a Geissoschizine synthase (GS);
   a Geissoschizine oxidase (GO);
   a Redox1;
   a Redox2;
   a Stemmadenine O-acetyltransferase (SAT);
   a O-acetylstemmadenine oxidase (PAS);
   a Dehydroprecondylocarpine acetate synthase (DPAS);
   a Tabersonine synthase (TS); and/or
   a Catharanthine synthase (CS),
   wherein preferably said CPR is CroCPR, said CYB5is CroCYB5, said GS is CroSG, said GO is CroGO, said Redox1 is CroRedox1, said Redox2 is CroRedox2, said SAT is CroSAT, said PAS is CroPAS, said DPAS is CroDPAS, said TS is CroTS and/or said CS is CroCS or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 and/or SEQ ID NO: 41, respectively.
   wherein the microorganism is capable of producing tabersonine and/or catharanthine, optionally wherein said method further comprises the step of recovering tabersonine and/or catharanthine.
43. The method according to any one of items 28 to 42, wherein the medium comprises at least strictosidine, preferably at a concentration of at least 0.05 mM, such as at least 0.1 mM, such as at least 0.5 mM, such as at least 1 mM.
44. The method according to any one of items 288 to 43, wherein the medium comprises at least tryptamine and secologanin, preferably at a concentration of at least 0.05 mM, such as at least 0.1 mM, such as at least 0.5 mM, such as at least 1 mM.
45. A nucleic acid construct comprising a sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 1, SEQ ID NO: 2, SEQ ID NO: 3,SEQ ID NO: 4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:100, SEQ ID NO:101, SEQ ID NO:102, SEQ ID NO:103, SEQ ID NO:104, SEQ ID NO:105, SEQ ID NO:106 and/or SEQ ID NO:107.
46. The nucleic acid construct according to item 45, further comprising a sequence identical to or having at 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 7.
47. The nucleic acid construct according to any of items 45 to 46, further comprising a sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 5 and/or SEQ ID NO: 23.
48. The nucleic acid construct according to any of items 45 to 47, further comprising a nucleic acid sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 6.
49. The nucleic acid construct according to any one of items 45 to 48, further comprising a nucleic acid sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and/or SEQ ID NO: 18.
50. The nucleic acid construct according to any of items 45 to 49, wherein at least one of the one or more nucleic acid sequences are under the control of an inducible promoter.
51. The nucleic acid construct according to any of items 45 to 50, wherein the nucleic acid construct is a vector such as an integrative vector or a replicative vector.
52. A vector comprising a nucleic acid sequence as defined in any one of items 45 to 50.
53. A host cell comprising one or more nucleic acid sequence as defined in any of items 45 to 50, or the vector according to item 52.
54. A kit of parts comprising a microorganism according to any one of items 1 to 36, and/or nucleic acid constructs according to any one of items 45 to 50, and/or a vector according to item 52, and instructions for use.
55. Use of the nucleic acid construct according to any one of items 45 to 50, of the microorganism according to any of items 1 to 36, the vector according to item 52, or the host cell according to item 53, for the production of strictosidine aglycone and/or tetrahydroalstonine, alstonine, tabersonine and/or catharanthine in a microorganism.
56. The use according to item 55 in the method according to items 37 to 44.
57. Strictosidine aglycone obtained by the method according to any of items 37 to 44.
58. Tetrahydroalstonine obtained by the method according to any of items 39 to 44.
59. Heteroyohimbine obtained by the method according to any of items 41 to 44.
60. Tabersonine and/or catharanthine obtained by the method according item 42 to 44.

61. A method of producing monoterpenoid indole alkaloids (MIAs) in a microorganism, said method comprising the steps of:
a) providing a microorganism capable of converting strictosidine to tabersonine and/or catharanthine, said cell expressing:
   a strictosidine-beta-glucosidase (SGD);
   a NADPH-cytochrome P450 reductase (CPR);
   a Cytochrome b5 (CYB5);
   a Geissoschizine synthase (GS);
   a Geissoschizine oxidase (GO);
   a Redox1;
   a Redox2;
   a Stemmadenine O-acetyltransferase (SAT);
   a O-acetylstemmadenine oxidase (PAS);
   a Dehydroprecondylocarpine acetate synthase (DPAS);
   a Tabersonine synthase (TS); and/or
   a Catharanthine synthase (CS);
   optionally, a strictosidine synthase (STR);
b) incubating said microorganism in a medium comprising strictosidine or a substrate which can be converted to strictosidine by said microorganism;
c) optionally, recovering the MIAs;
d) optionally, processing the MIAs into a pharmaceutical compound,
wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
and/or;
wherein said SGD is a mosaic SGD, wherein said mosaic SGD comprises an amino acid sequence having the general formula

D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,

wherein said first SGD, second SGD and fourth SGD can be the same or different, with the proviso that said first SGD, second SGD and fourth SGD are not all RseSGD.
62. The method according to item 61, wherein the microorganism is as defined in any one of the preceding items.
63. A method of treating a disorder such as a cancer, arrhythmia, malaria, psychotic diseases, hypertension, depression, Alzheimer's disease, addiction and/or neuronal diseases, comprising administration of a therapeutic sufficient amount of an MIA or a pharmaceutical compound obtained by the method according to any of items 24 to 30, 47 or 61 to 62.

1. A microorganism capable of producing strictosidine aglycone, said microorganism expresses
a strictosidine-beta-glucosidase (SGD), capable of converting strictosidine to strictosidine aglycone,
wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
and/or;
wherein said SGD is a mosaic SGD, wherein said mosaic SGD comprises an amino acid sequence having the general formula
D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,
wherein said first SGD, second SGD and fourth SGD can be the same or different, with the proviso that said first SGD, second SGD and fourth SGD are not all RseSGD.

2. The microorganism according to claim 1, wherein the microorganism is selected from the group consisting of bacteria, archaea, yeast, fungi, protozoa, algae, and viruses, preferably the microorganism is a yeast or a bacteria, such as Saccharomyces cerevisiae or Escherichia coli.

3. The microorganism according to any one the preceding claims, further expressing
a strictosidine synthase (STR), capable of converting secologanin and tryptamine to strictosidine, whereby the microorganism is capable of synthesising strictosidine,
wherein said STR is preferably CroSTR or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 30.

4. The microorganism according to any one of the preceding claims, wherein D₁ comprises or consists of an amino acid sequence corresponding to amino acids M1 to R115 of SEQ ID NO:24.

5. The microorganism according to any one of the preceding claims, wherein D₂ comprises or consists of an amino acid sequence corresponding to amino acids F116 to G266 of SEQ ID NO:24.

6. The microorganism according to any one of the preceding claims, wherein D₄ comprises or consists of amino acids of SEQ ID NO:92 or a variant thereof having at least 90% identity to SEQ ID NO: 92.

7. The microorganism according to any one of the preceding claims, wherein at least one of D₁, D₂ or D₄ is from an SGD which is native to a first organism selected from Gelsemium sempervirens, Scedosporium apiospermum or Rauvolfia verticillata, Vinca minor, Tabernaemontana elegans, Amsonia hubrichtii, Ophiorrhiza pumila, Nyssa sinensis, Coffea arabica, Carapichea ipecacuanha, Handroanthus impetiginosus, Sesamum indicum, Actinidia chinensis var. chinensis, Helianthus annuus, Lactuca sativa, Ipomoea nil, Vigna unguiculata, Heliocybe sulcate, Pyricularia grisea, Lomentospora prolificans, Hydnomerulius pinastri MD-312, and Moniliophthora roreri MCA 2997.

8. The microorgagnism according to any one of the preceding claims, wherein the first SGD, the second SGD and the fourth SGD are identical or different.

9. The microorganism according to any one of the preceding claims, wherein two of the first SGD, the second SGD and the fourth SGD are identical, or wherein the first SGD, the second SGD and the fourth SGD are different, or wherein the first SGD, the second SGD and the fourth SGD are identical.

10. The microorganism according to any one of the preceding claims, wherein said mosaic SGD comprises or consists of an amino acid sequence of SEQ ID NO: 93, SEQ ID NO: 94, SEQ ID NO: 95, SEQ ID NO: 96, SEQ ID NO: 97, SEQ ID NO: 98, SEQ ID NO: 99 or SEQ ID NO: 8, or variants thereof having at least 90% identity or homology thereto, such as at least 91%, such as at least 92%, such as at least 93%, such as at least 94%, such as at least 95%, such as at least 96%, such as at least 97%, such as at least 98%, such as at least 99% identity or homology thereto.

11. The microorganism according to any one the preceding claims, further expressing:
i. a tetrahydroalstonine synthase (THAS) and/or a heteroyohimbine synthase (HYS), capable of converting strictosidine aglycone to tetrahydroalstonine, whereby the microorganism is capable of synthesising tetrahydroalstonine,
wherein said THAS is preferably CroTHAS and/or HYS is CroHYS or variants thereof, having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 28 and/or SEQ ID NO: 46,
and optionally further expressing
a sarpargan bridge enzymes (SBE), capable of converting tetrahydroalstonine and ajmalicine to a heteroyohimbine selected from the group consisting of alstonine and serpentine, whereby the microorganism is capable of synthesising alstonine and serpentine,
wherein said SBE is preferably GseSBE or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 29,
and/or
ii. further expressing
a NADPH-cytochrome P450 reductase (CPR);
a Cytochrome b5 (CYB5);
a Geissoschizine synthase (GS);
a Geissoschizine oxidase (GO);
a Redox1;
a Redox2;
a Stemmadenine O-acetyltransferase (SAT);
a O-acetylstemmadenine oxidase (PAS);
a Dehydroprecondylocarpine acetate synthase (DPAS);
a Tabersonine synthase (TS); and/or
a Catharanthine synthase (CS),
whereby the microorganism is capable of synthesising tabersonine and/or catharanthine,
wherein preferably said CPR is CroCPR, said CYB5is CroCYB5, said GS is CroSG, said GO is CroGO, said Redox1 is CroRedox1, said Redox2 is CroRedox2, said SAT is CroSAT, said PAS is CroPAS, said DPAS is CroDPAS, said TS is CroTS and/or said CS is CroCS or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 and/or SEQ ID NO: 41, respectively.

12. The microorganism according to any one of the preceding claims, capable of producing strictosidine aglycone with a titre of at least 1 μM, such as at least 2 μM, such as at least 4 μM, such as at least 6 μM, such as at least 8 μM such as at least 10 μM or more.

13. The microorganism according to claim 11, capable of producing:
i. tetrahydroalstonine with a titre of at least 1 μM, such as at least 2 μM, such as at least 4 μM, such as at least 6 μM, such as at least 8 μM such as at least 10 μM or more, and optionally alstonine with a titre of at least 1 μM, such as at least 2 μM, such as at least 4 μM, such as at least 6 μM, such as at least 8 μM such as at least 10 μM or more, and/or
ii. tabersonine with a titre of at least 0.01 μM, such as at least 0.02 μM, and/or catharanthine with a titre of at least 0.01 μM, such as at least 0.02 μM.

14. A method of producing strictosidine aglycone in a microorganism, said method comprises the steps of:
a) providing a microorganism, said cell expressing:
a strictosidine-beta-glucosidase (SGD), capable of converting strictosidine to strictosidine aglycone;
b) incubating said microorganism in a medium comprising strictosidine or a substrate which can be converted to strictosidine by said microorganism;
c) optionally, recovering the strictosidine aglycone;
d) optionally, further converting the strictosidine aglycone to monoterpenoid indole alkaloids,
wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
and/or;
wherein said SGD is a mosaic SGD, wherein said mosaic SGD comprises an amino acid sequence having the general formula
D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,
wherein said first SGD, second SGD and fourth SGD can be the same or different, with the proviso that said first SGD, second SGD and fourth SGD are not all RseSGD.

15. The method according to claim 14, wherein the SGD, the heterologous SGD and/or the mosaic SGD is as defined in any one of claims 1 to 13.

16. The method according to any one of claims 14 to 15, wherein the substrate is secologanin and/or tryptamine, and wherein said microorganism further expresses:
a strictosidine synthase (STR), capable of converting secologanin and tryptamine to strictosidine;
wherein said STR is preferably CroSTR or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 30.

17. The method according to any one of claims 14 to 16, wherein the method comprises step d) and wherein said microorganism further expresses:
i. a tetrahydroalstonine synthase (THAS) and/or or a heteroyohimbine synthase (HSY), capable of converting strictosidine aglycone to tetrahydroalstonine;
wherein preferably said THAS is identical to or has at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 28 and/or HYS is identical to or has at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 46, optionally wherein said method further comprises the step of recover tetrahydroalstonine, and optionally wherein said microorganism further expresses:
a sapargan bridge enzyme (SBE), capable of converting tetrahydroalstonine to alstonine;
wherein preferably said SBE is identical to or has at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 29, optionally wherein said method further comprises the step of recovering alstonine, and/or
ii. wherein said microorganism further expresses:
a NADPH-cytochrome P450 reductase (CPR);
a Cytochrome b5 (CYB5);
a Geissoschizine synthase (GS);
a Geissoschizine oxidase (GO);
a Redox1;
a Redox2;
a Stemmadenine O-acetyltransferase (SAT);
a O-acetylstemmadenine oxidase (PAS);
a Dehydroprecondylocarpine acetate synthase (DPAS);
a Tabersonine synthase (TS); and/or
a Catharanthine synthase (CS),
wherein preferably said CPR is CroCPR, said CYB5 is CroCYB5, said GS is CroSG, said GO is CroGO, said Redox1 is CroRedox1, said Redox2 is CroRedox2, said SAT is CroSAT, said PAS is CroPAS, said DPAS is CroDPAS, said TS is CroTS and/or said CS is CroCS or variants thereof having at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 and/or SEQ ID NO: 41, respectively,
wherein the microorganism is capable of producing tabersonine and/or catharanthine, optionally wherein said method further comprises the step of recovering tabersonine and/or catharanthine.

18. A nucleic acid construct comprising a sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 1, SEQ ID NO: 2, SEQ ID NO: 3, SEQ ID NO: 4, SEQ ID NO:68, SEQ ID NO:69, SEQ ID NO:70, SEQ ID NO: 71, SEQ ID NO:72, SEQ ID NO: 73, SEQ ID NO:74, SEQ ID NO: 75, SEQ ID NO: 76, SEQ ID NO: 77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:100, SEQ ID NO:101, SEQ ID NO:102, SEQ ID NO:103, SEQ ID NO:104, SEQ ID NO:105, SEQ ID NO:106 and/or SEQ ID NO:107, optionally, further comprising a sequence identical to or having at 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 7.

19. The nucleic acid construct according to claim 18, further comprising a sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 5 and/or SEQ ID NO: 23, and/or optionally further comprising a nucleic acid sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 6, and/or further comprising a nucleic acid sequence identical to or having at least 90% identity, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and/or SEQ ID NO: 18.

20. A vector comprising a nucleic acid sequence as defined in any one of claims 18 to 19.

21. A host cell comprising one or more nucleic acid sequence as defined in any one of claims 18 to 19, or the vector according to claim 20.

22. A kit of parts comprising a microorganism according to any one of claims 1 to 13, and/or nucleic acid constructs according to any one of claims 18 to 19, and/or a vector according to claim 20, and instructions for use.

23. Use of the nucleic acid construct according to any one of claims 18 to 19, of the microorganism according to any of claims 1 to 13, the vector according to claim 20, or the host cell according to claim 21, for the production of strictosidine aglycone, tetrahydroalstonine, alstonine, tabersonine and/or catharanthine in a microorganism, preferably according to the method in claims 14 to 17.

24. A method of producing monoterpenoid indole alkaloids (MIAs) in a microorganism, said method comprising the steps of:
a) providing a microorganism capable of converting strictosidine to tabersonine and/or catharanthine, said cell expressing:
a strictosidine-beta-glucosidase (SGD);
a NADPH-cytochrome P450 reductase (CPR);
a Cytochrome b5 (CYB5);
a Geissoschizine synthase (GS);
a Geissoschizine oxidase (GO);
a Redox1;
a Redox2;
a Stemmadenine O-acetyltransferase (SAT);
a O-acetylstemmadenine oxidase (PAS);
a Dehydroprecondylocarpine acetate synthase (DPAS);
a Tabersonine synthase (TS); and/or
a Catharanthine synthase (CS);
b) incubating said microorganism in a medium comprising strictosidine or a substrate which can be converted to strictosidine by said microorganism;
c) optionally, recovering the MIAs;
d) optionally, processing the MIAs into a pharmaceutical compound,
wherein said SGD is a heterologous SGD selected from RseSGD (SEQ ID NO: 24), GseSGD (SEQ ID NO: 25), SapSGD (SEQ ID NO: 26), RveSGD (SEQ ID NO: 27), VmiSGD1 (SEQ ID NO: 47), AhuSGD (SEQ ID NO: 48), HimSGD2 (SEQ ID NO: 49), SinSGD (SEQ ID NO: 50), TelSGD (SEQ ID NO: 51), VunSGD (SEQ ID NO: 52), NsiSGD1 (SEQ ID NO: 53), LprSGD (SEQ ID NO: 54), AchSGD1 (SEQ ID NO: 55), HsuSGD (SEQ ID NO: 56), MroSGD (SEQ ID NO: 57), RseSGD2 (SEQ ID NO: 58), PgrSGD (SEQ ID NO: 59), OpuSGD (SEQ ID NO: 60), HpiSGD (SEQ ID NO: 61), HanSGD1 (SEQ ID NO: 62), AchSGD2 (SEQ ID NO: 63), HimSGD1 (SEQ ID NO: 64), IpeSGD (SEQ ID NO: 65), LsaSGD1 (SEQ ID NO: 66), or CarSGD (SEQ ID NO: 67) or variants thereof having at least 70%, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto,
and/or;
wherein said SGD is a mosaic SGD, wherein said mosaic SGD comprises an amino acid sequence having the general formula
D₁-D₂-D₃-D₄
wherein D₁ is a first amino acid sequence from a first SGD,
wherein D₂ is a second amino acid sequence from a second SGD,
wherein D₃ is a third amino acid sequence comprising or consisting of amino acids of SEQ ID NO:91 or a variant thereof having at least 90% identity to SEQ ID NO: 91,
wherein D₄ is a fourth amino acid sequence from a fourth SGD or an amino acid sequence consisting of amino acids of SEQ ID NO:92 or a variant thereof having at least 90% identity to SEQ ID NO: 92,
wherein said first SGD, second SGD and fourth SGD can be the same or different, with the proviso that said first SGD, second SGD and fourth SGD are not all RseSGD.

25. The method according to claim 24, wherein said microorganism further expresses strictosidine (STR).

26. The method according to any one of claims 24-26, wherein said microorganism is as defined in any one of claims 1 to 14.
